(19) United States
(12) Patent Application Publication
(10) Pub. No.: US 2025/0027093 A1
(43) Pub. Date: Jan. 23, 2025

(54) MOLECULAR SWITCHES

(71) Applicant: PRECIGEN, INC., Germantown, MD (US)

(72) Inventors: Mark Anton HELD, Germantown, MD (US); Xinhua ZHAO, Germantown, MD (US); Lily Yuin CHAO, Germantown, MD (US); Na TRINH, Germantown, MD (US); James KEALEY, Germantown, MD (US); Kevin Lee DIETZEL, Germantown, MD (US)

(73) Assignee: PRECIGEN, INC., Germantown, MD (US)

(21) Appl. No.: 18/805,099

(22) Filed: Aug. 14, 2024

Related U.S. Application Data
(62) Division of application No. 17/454,060, filed on Nov. 8, 2021, now Pat. No. 12,091,667.
Application No. 17/454,060 is a division of application No. 16/481,811, filed on Jul. 29, 2019, now Pat. No. 11,198,877.
(60) Provisional application No. 62/588,985, filed on Nov. 21, 2017.
(60) Provisional application No. 62/512,312, filed on May 30, 2017.
(60) Provisional application No. 62/504,626, filed on May 11, 2017.
(60) Provisional application No. 62/451,819, filed on Jan. 30, 2017.

(51) Int. Cl. C12N 15/52; C12N 1/20; C12N 9/04; C12N 9/10; C12N 9/88; C12N 15/74; C12P 5/02; C12P 7/16; C12P 7/18

(52) U.S. Cl. CPC C12N 15/52; C12N 1/20; C12N 9/0006; C12N 9/1022; C12N 9/88; C12N 15/74; C12P 5/023; C12P 7/16; C12P 7/18; C12Y 101/01304; C12Y 202/01006; C12Y 401/01004; C12Y 401/01005

(57) ABSTRACT

Genetically modified microorganisms that have the ability to convert carbon substrates into chemical products such as 2,3-BDO; 1,4-BDO; isobutyraldehyde; isobutanol; 1-butanol; n-butanol; ethanol; fatty alcohols; and fatty acid methyl ester are disclosed. For example, genetically modified methanotrophs that are capable of generating 2,3-BDO; 1,4-BDO; isobutyraldehyde; isobutanol; 1-butanol; n-butanol; ethanol; fatty alcohols; and fatty acid methyl ester at high titers from a methane source are disclosed. Methods of making these genetically modified microorganisms and methods of using them are also disclosed. These microorganisms and methods make use of molecular switches to regulate gene expression.

### CROSS-REFERENCE TO RELATED APPLICATION

This application is a divisional of U.S. application Ser. No. 17/454,060, filed Nov. 8, 2021, which in turn is a divisional of U.S. application Ser. No. 16/481,811, issued on Dec. 14, 2021 as U.S. Pat. No. 11,198,877, which is a U.S. national stage filing of International Application No. PCT/US2018/015925, filed Jan. 30, 2018, which claims priority benefit of U.S. Provisional Application No. 62/451,819, filed Jan. 30, 2017; 62/504,626, filed May 11, 2017; 62/512,312, filed May 30, 2017; and 62/588,985, filed Nov. 21, 2017, which are hereby incorporated by reference in their entireties.

### REFERENCE TO SEQUENCE LISTING

This application contains a Sequence Listing, which has been submitted electronically in XML format and is hereby incorporated by reference in its entirety. The XML copy was created on Aug. 5, 2024, is named 17454060CON.xml and is 250,402 bytes in size.

### BACKGROUND OF THE INVENTION

Microbial fermentation of valued chemicals sometimes requires the ability to control the expression of certain genes to efficiently produce the valued chemicals. For example, the ability to reversibly “turn on” or “turn off” expression of certain genes during fermentation by adding substances directly to the fermentation tank can extremely beneficial and can speed the up the fermentation process while decreasing costs.

Here we describe microorganisms and methods that can be used to efficiently produce valued chemicals (e.g., multicarbon products). For example, we described genetically modified microorganisms and methods of fermenting them in such a way that the addition or removal of a particular substance can effectively and efficiently “switch” on or off certain genes, which can ultimately lead to valued chemical production.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

### SUMMARY

Disclosed herein are methods of regulating gene expression using a molecular switch in genetically modified microorganisms that are capable of producing a desired organic compound from a carbon source, such as a single carbon containing hydrocarbon molecule such as methane. Various genetically modified microorganisms, vectors, and methods are disclosed throughout.

For example, disclosed herein is a genetically modified microorganism capable of converting a C1 carbon into a multicarbon product, where the microorganism comprises a heterologous gene under the control of a molecular switch.

In some cases, the molecular switch can be responsive to a sugar or rare earth metal. For example, if the molecular switch is responsive to a sugar, the sugar can be arabinose. The molecular switch can be responsive to arabinose at a concentration of at least 0.1 mM, e.g., 1 mM. In some cases, the molecular switch can be response to another type of sugar, isopropyl β-D-1-thiogalactopyranoside (IPTG). The molecular switch can be responsive to IPTG at a concentration of at least 1 μM, e.g., 10 μM.

In some cases, the molecular switch can be responsive to a rare earth metal. The rare earth metal can be cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), scandium (Sc), terbium (Tb), thulium (Tm), ytterbium (Yb), yttrium (Y), or any combination thereof.

In some cases, the molecular switch is responsive to the rare earth metal cerium (Ce). In some cases, the molecular switch is responsive to cerium (Ce) at a concentration of at least 10 μM. In some cases, maximal response to cerium (Ce) can occur at a concentration of at least 70 μM.

In some cases, the molecular switch is responsive to the rare earth metal lanthanum (La). In some cases, the molecular switch is responsive to lanthanum (La) at a concentration of at least 0.5 μM. In some cases, maximal response to lanthanum (La) can occur at a concentration of at least 35 μM.

In some cases, the molecular switch is responsive to the rare earth metal praseodymium (Pr). In some cases, the molecular switch is responsive to praseodymium (Pr) at a concentration of at least 10 μM. In some cases, maximal response to praseodymium (Pr) can occur at a concentration of at least 140 μM.

In some cases, the molecular switch is responsive to the rare earth metal neodymium (Nd). In some cases, the molecular switch is responsive to neodymium (Nd) at a concentration of at least 10 μM. In some cases, maximal response to neodymium (Nd) can occur at a concentration of at least 70 μM.

In some cases, the expression of the heterologous gene is induced by the molecular switch. In some cases, the expression of the heterologous gene is repressed by the molecular switch. In some cases, the expression of the expression of the heterologous gene is reversed by dilution of the medium.

In some cases, the expression of the heterologous gene is repressed by at least 50%. In some cases, the expression of the heterologous gene is repressed by at least 75%. In some cases, the expression of the heterologous gene is repressed by at least 90%. In some cases, the expression of the heterologous gene is de-repressed by dilution of the medium. In some cases, the dilution is at least a 1.1× dilution.

The genetically modified microorganism can be, in some instances, a methanotroph. The methanotroph can be from the genera Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, or Methyloacidophilum. For example, the methanotroph is a Methylococcus or more specifically the methanotroph is a Methylococcus capsulatus.

In some cases, the genetically modified microorganism produces a multicarbon product that is 2,3-butanediol; 1,4-butanediol; isobutyraldehyde; isobutanol; 1-butanol; n-butanol; ethanol; fatty (or aliphatic long chain) alcohols; fatty acid methyl esters, or any combination thereof.

When the genetically modified microorganism produces a multicarbon product that is 2,3-butanediol, the genetically modified microorganism can comprises a heterologous gene under the control of a molecular switch, where the heterologous gene is acetolactate synthase, alpha-acetolactate decarboxylase, acetoin reductase, or any combination thereof. Should an acetolactate synthase be used, it can be in some cases encoded by a polynucleotide that is substantially similar to any one of SEQ ID NOs: 2, 4, or 6. Should an alpha-acetolactate decarboxylase be used, it can be in some cases encoded by a polynucleotide that is substantially similar to SEQ ID NO: 8 or 10. Should an acetoin reductase be used, it can be in some cases encoded by a polynucleotide that is substantially similar to SEQ ID NO: 12.

When the genetically modified microorganism produces a multicarbon product that is 1,4-butanediol, the genetically modified microorganism can comprises a heterologous gene under the control of a molecular switch, where the heterologous gene is succinyl-CoA synthetase (sucC), CoA-dependent succinate semialdehyde dehydrogenase (sucD), 4-hydroxybutyrate dehydrogenase (4hbD), 4-hydroxybutyryl-CoA transferase (cat2), aldehyde dehydrogenase (ald), alcohol dehydrogenase (adh), or any combination thereof. Should a succinyl-CoA synthetase be used, it can be in some cases encoded by a polynucleotide that is substantially similar to SEQ ID NO: 18. Should a CoA-dependent succinate semialdehyde dehydrogenase be used, it can be in some cases encoded by a polynucleotide that is substantially similar to SEQ ID NO: 20. Should a 4-hydroxybutyrate dehydrogenase be used, it can be in some cases encoded by a polynucleotide that is substantially similar to SEQ ID NO: 22. Should a 4-hydroxybutyryl-CoA transferase be used, it can be in some cases encoded by a polynucleotide that is substantially similar to SEQ ID NO: 24. Should an aldehyde dehydrogenase be used, it can be in some cases encoded by a polynucleotide that is substantially similar to SEQ ID NO: 26. Should an alcohol dehydrogenase be used, it can be in some cases encoded by a polynucleotide that is substantially similar to SEQ ID NO: 28.

When the genetically modified microorganism produces a multicarbon product that is isobutyraldehyde, the genetically modified microorganism can comprises a heterologous gene under the control of a molecular switch, where the heterologous gene is acetolactate synthase (AlsS); ketol-acid reductoisomerase (KARI); dihydroxy-acid dehydratase (DHAD); 2-keto acid decarboxylase (KDC), or any combination thereof. Should an acetolactate synthase be used, it can be in some cases encoded by a polynucleotide that is substantially similar to SEQ ID NO: 30. Should a ketol-acid reductoisomerase be used, it can be in some cases encoded by a polynucleotide that is substantially similar to SEQ ID NO: 32. Should a dihydroxy-acid dehydratase be used, it can be in some cases encoded by a polynucleotide that is substantially similar to SEQ ID NO: 34. Should a 2-keto acid decarboxylase be used, it can be in some cases encoded by a polynucleotide that is substantially similar to any one of SEQ ID NOs: 36, 38, 40, 42, 44, 46 or 48.

When the genetically modified microorganism produces a multicarbon product that is isobutanol, the genetically modified microorganism can comprises a heterologous gene under the control of a molecular switch, where the heterologous gene is acetolactate synthase (AlsS); ketol-acid reductoisomerase (KARI); dihydroxy-acid dehydratase (DHAD); 2-keto acid decarboxylase (KDC), alcohol dehydrogenase (ADH) or any combination thereof. Should an acetolactate synthase be used, it can be in some cases encoded by a polynucleotide that is substantially similar to SEQ ID NO: 30. Should a ketol-acid reductoisomerase be used, it can be in some cases encoded by a polynucleotide that is substantially similar to SEQ ID NO: 32. Should a dihydroxy-acid dehydratase be used, it can be in some cases encoded by a polynucleotide that is substantially similar to SEQ ID NO: 34. Should a 2-keto acid decarboxylase be used, it can be in some cases encoded by a polynucleotide that is substantially similar to any one of SEQ ID NOs: 36, 38, 40, 42, 44, 46 or 48. Should an alcohol dehydrogenase be used, it can be in some cases encoded by a polynucleotide that is substantially similar to any one of SEQ ID NO: 50, 52, 54, 56 or 58.

When the genetically modified microorganism produces a multicarbon product that is 1-butanol, the genetically modified microorganism can comprises a heterologous gene under the control of a molecular switch, where the heterologous gene is L-threonine ammonia lyase, 2-ethylmalate synthase, isopropylmalate isomerase, 2-ketoacid decarboxylase, alcohol dehydrogenase, or any combination thereof. Should an L-threonine ammonia lyase be used, it can be in some cases encoded by a polynucleotide that is substantially similar to SEQ ID NO: 60. Should a 2-ethylmalate synthase be used, it can be in some cases encoded by a polynucleotide that is substantially similar to SEQ ID NO: 62. Should an isopropylmalate isomerase be used, it can be in some cases encoded by a polynucleotide that is substantially similar to SEQ ID NO: 64 or 66. Should a 2-ketoacid decarboxylase be used, it can be in some cases encoded by a polynucleotide that is substantially similar to any one of SEQ ID NOs: 36, 38, 40, 42, 44, 46 or 48. Should an alcohol dehydrogenase be used, it can be in some cases encoded by a polynucleotide that is substantially similar to any one of SEQ ID NOs: 50, 52, 54, 56 or 58.

When the genetically modified microorganism produces a multicarbon product that is ethanol, the genetically modified microorganism can comprises a heterologous gene under the control of a molecular switch, where the heterologous gene is pyruvate decarboxylase (PDC), alcohol dehydrogenase (ADH), or any combination thereof. Should a pyruvate decarboxylase be used, it can be in some cases encoded by a polynucleotide that is substantially similar to SEQ ID NO: 68. Should an alcohol dehydrogenase be used, it can be in some cases encoded by a polynucleotide that is substantially similar to SEQ ID NO: 70.

When the genetically modified microorganism produces a multicarbon product that is fatty alcohol, the genetically modified microorganism can comprises a heterologous gene under the control of a molecular switch, where the heterologous gene is fatty-acyl-CoA reductase (FAR). In these cases, the fatty-acyl-CoA reductase (FAR) can be encoded by a polynucleotide that is substantially similar to SEQ ID NO: 72

When the genetically modified microorganism produces a multicarbon product that is fatty acid methyl ester, the genetically modified microorganism can comprises a heterologous gene under the control of a molecular switch, where the heterologous gene is a WES polypeptide. In these cases, the WES polypeptide can be encoded by a polynucleotide that is substantially similar to any one of SEQ ID NOs: 74, 76, 78, 80, or 82.

In some cases, the C1 carbon is carbon monoxide (CO), carbon dioxide (CO₂), methane (CH₄), or any combination thereof.

Further disclosed herein is a vector comprising a promoter driving the expression of a gene, where the promoter is responsive to a rare earth metal and is active in a methanotroph.

The rare earth metal can be any disclosed throughout, such as Ce, Dy, Er, Eu, Gd, Ho, La, Lu, Nd, Pr, Pm, Sm, Sc, Tb, Tm, Yb, Y, or any combination thereof.

In some cases, the rare earth metal is cerium. In these cases, the promoter can be responsive to cerium (Ce) at a concentration of at least 10 μM. In some case, the rare earth metal is lanthanum (La). In these cases, the promoter can be responsive to lanthanum (La) at a concentration of at least 0.5 μM. In some case, the rare earth metal is praseodymium (Pr). In these cases, the promoter can be responsive to praseodymium (Pr) at a concentration of at least 10 μM. In some case, the rare earth metal is neodymium (Nd). In these cases, the promoter can be responsive to neodymium (Nd) at a concentration of at least 10 μM.

In some cases, the vector can further comprising a gene. In some cases, the gene can be acetolactate synthase, alpha-acetolactate decarboxylase, acetoin reductase, or any combination thereof. In other cases, the gene can be succinyl-CoA synthetase (sucC), CoA-dependent succinate semialdehyde dehydrogenase (sucD), 4-hydroxybutyrate dehydrogenase (4hbD), 4-hydroxybutyryl-CoA transferase (cat2), aldehyde dehydrogenase (ald), alcohol dehydrogenase (adh), or any combination thereof. In some cases, the gene can be acetolactate synthase (AlsS); ketol-acid reductoisomerase (KARI); dihydroxy-acid dehydratase (DHAD); 2-keto acid decarboxylase (KDC), or any combination thereof. In some cases, the gene can be acetolactate synthase (AlsS); ketol-acid reductoisomerase (KARI); dihydroxy-acid dehydratase (DHAD); 2-keto acid decarboxylase (KDC), alcohol dehydrogenase (ADH), or any combination thereof. In some cases, the gene can be L-threonine ammonia lyase, 2-ethylmalate synthase, isopropylmalate isomerase, 2-ketoacid decarboxylase, alcohol dehydrogenase, or any combination thereof. In some cases, the gene can be pyruvate decarboxylase (PDC), alcohol dehydrogenase (ADH), or any combination thereof. In some cases, the gene can be fatty-acyl-CoA reductase (FAR). In some cases, the gene can be a WES polypeptide.

Additionally disclosed is a method of making a genetically modified microorganism capable of converting a C1 carbon to a multicarbon product comprising transforming a microorganism with the vector as described throughout. In some of these cases, the genetically modified microorganism is capable of producing 2,3-butanediol; 1,4-butanediol; isobutyraldehyde; isobutanol; 1-butanol; n-butanol; ethanol; fatty (or aliphatic long chain) alcohols; and/or fatty acid methyl esters.

Further disclosed herein is a method of making a multicarbon product from a C1 carbon comprising (a) contacting the genetically modified microorganism as described throughout with a C1 carbon; and (b) growing the microorganism to produce the multicarbon product. In some cases, the multicarbon product is 2,3-butanediol; 1,4-butanediol; isobutyraldehyde; isobutanol; 1-butanol; n-butanol; ethanol; fatty (or aliphatic long chain) alcohols; and/or fatty acid methyl esters. In some cases, the C1 carbon is methane. In some cases, the multicarbon product produced is substantially pure. In some cases, the method can further comprising recovering the multicarbon product. In some cases, the method can further comprise contacting the multicarbon product with a catalyst to produce a different product. In some cases, the multicarbon product can be 2,3-BDO. In some cases, the different product can be butadiene. In some cases, the catalyst is a SiO₂-supported cesium dihydrogen phosphate (CsH₂PO₄) catalyst. In some cases, the different product is methyl ethyl ketone. In some cases, the catalyst is a solid acid catalyst.

In some cases, the method can further comprise contacting the microorganism with a sugar that can activate or repress a molecular switch. The sugar can be within media. In some cases, the sugar can be arabinose. If arabinose is used, the arabinose can be present at a concentration of at least 0.1, e.g., 6.6 mM. In some cases, the sugar can be isopropyl β-D-1-thiogalactopyranoside (IPTG). When IPTG is used, it can be present at a concentration of at least 1 μM, e.g., 10 μM.

In some cases, the method can further comprise contacting the microorganism with media having a rare earth metal that can activate or repress a molecular switch. The rare earth metal can be any disclosed throughout, such as Ce, Dy, Er, Eu, Gd, Ho, La, Lu, Nd, Pr, Pm, Sm, Sc, Tb, Tm, Yb, Y, or any combination thereof. In some cases, the rare earth metal is cerium (Ce). When cerium is the rare earth metal, it can be present at a concentration of at least 10 μM. In some cases, the rare earth metal is lanthanum (La). When lanthanum (La) is the rare earth metal, it can be present at a concentration of at least 0.5 μM. When praseodymium (Pr) is the rare earth metal, it can be present at a concentration of at least 10 μM. When neodymium (Nd) is the rare earth metal, it can be present at a concentration of at least 10 μM.

Also disclosed herein is a method of altering the expression of a gene or set of genes within a microorganism comprising contacting the microorganism with a rare earth metal, where the gene or set of genes are comprised in a heterologous polynucleotide having a molecular switch that is responsive to the rare earth metal. In some cases, the gene or set of genes is acetolactate synthase, alpha-acetolactate decarboxylase, acetoin reductase, or any combination thereof. In some cases, the gene or set of genes is succinyl-CoA synthetase (sucC), CoA-dependent succinate semialdehyde dehydrogenase (sucD), 4-hydroxybutyrate dehydrogenase (4hbD), 4-hydroxybutyryl-CoA transferase (cat2), aldehyde dehydrogenase (ald), alcohol dehydrogenase (adh), or any combination thereof. In some cases, the gene or set of genes is acetolactate synthase (AlsS); ketol-acid reductoisomerase (KARI); dihydroxy-acid dehydratase (DHAD); 2-keto acid decarboxylase (KDC), or any combination thereof. In some cases, the gene or set of genes is acetolactate synthase (AlsS); ketol-acid reductoisomerase (KARI); dihydroxy-acid dehydratase (DHAD); 2-keto acid decarboxylase (KDC); alcohol dehydrogenase (ADH), or any combination thereof. In some cases, the gene or set of genes is L-threonine ammonia lyase, 2-ethylmalate synthase, isopropylmalate isomerase, 2-ketoacid decarboxylase, alcohol dehydrogenase, or any combination thereof. In some cases, the gene or set of genes is pyruvate decarboxylase (PDC), alcohol dehydrogenase (ADH), or any combination thereof. In some cases, the gene is fatty-acyl-CoA reductase (FAR). In some cases, the gene is a WES polypeptide.

The rare earth metal used in the method can be any disclosed throughout, such as Ce, Dy, Er, Eu, Gd, Ho, La, Lu, Nd, Pr, Pm, Sm, Sc, Tb, Tm, Yb, Y, or any combination thereof. In some cases, the rare earth metal is cerium (Ce). When cerium is the rare earth metal, it can be present at a concentration of at least 10 μM. In some cases, the rare earth metal is lanthanum (La). When lanthanum (La) is the rare earth metal, it can be present at a concentration of at least 0.5 μM. When praseodymium (Pr) is the rare earth metal, it can be present at a concentration of at least 10 μM. When neodymium (Nd) is the rare earth metal, it can be present at a concentration of at least 10 μM.

The molecular switch used in the method can comprise pBAD, PmxaF, pTRC, or pXoxF.

In some cases, the expression of the gene or set of genes is reduced upon contact with the rare earth metal. In some cases, the expression of the gene or set of genes is activated upon contact with the rare earth metal.

In some cases, the method further comprises diluting the rare earth metal. In some cases, the microorganism is in contact with the rare earth metal at all times before diluting the rare earth metal. For example, the microorganism is pre-cultured with the rare earth metal.

### BRIEF DESCRIPTION OF THE DRAWINGS

FIG. 1 shows the expression of many promoters within a methanotroph, including but not limited to the promoter pMxaF and J series promoters.

FIG. 2 shows the effect of lanthanum on pMxaF expression as measured by mCherry levels. In the presence of 35 μM lanthanum, expression is repressed by approximately 90%. After removal of the 35 μM lanthanum, approximately 50% activity is recovered.

FIG. 3 shows the effect of different concentrations of lanthanum on pMxaF expression as measured by mCherry levels. Maximal repression is seen at a concentration of above 8.8 μM lanthanum.

FIG. 4 shows the effect of de-repression after lanthanum treatment at various concentrations as measured by mCherry levels. Generally, the more lanthanum used to repress pMxaF, the less expression is recovered after lanthanum is removed from the system.

FIG. 5 shows that the pBAD promoter is turned on by the addition of 10 g/L of arabinose as measured by mCherry levels. Further, mCherry levels without any arabinose is identical to the levels seen in the background media. Also, in the presence of 35 μM lanthanum, there is still some background activity of pMxaF (compared to the media), indicating a bit of “leakiness.”

FIG. 6 shows that the pXoxF promoter is inducible in the presence of lanthanum to almost the same extent that pBAD is induced by arabinose. pXoxF in the absence of lanthanum does not exhibit any measurable leakiness.

FIG. 7 shows the ability of strains using a lanthanum response molecular switch to produce isobutanol. Four different strains having isobutanol enzymes driven by pMxaF showed production of isobutanol after lanthanum was diluted out from the initial medium.

FIGS. 8A and 8B. FIG. 8A demonstrates acetoin and 2,3-BDO production of 21 different strains after 96 hours following dilution of lanthanum containing media. The strains and genotypes are listed in Table 1 and 2. For strains 1 to 21, production titers were measured 96 hours after a 1:10 (10×) dilution of the culture into fresh medium whereas for strains 22 to 42 production titers were measured after a 1:50 (50×) dilution. Strains 22 to 27 produced high levels of 2,3-BDO, compared to strains subjected less dilution prior to the 2,3-BDO production phase. FIG. 8B demonstrates acetoin and 2,3-BDO production of 21 different strains after 120 hours following dilution of lanthanum containing media. The strains and genotypes of the stains are listed in Table 1 and 2. For strains 1 to 21, production titers were measured 120 hours after a 1:10 (10×) dilution of the culture into fresh medium, whereas for strains 22 to 42 production titers were measured after a 1:50 (50×) dilution. Strains 22 to 27 produced high levels of 2,3-BDO, compared to strains subjected to less dilution prior to the 2,3-BDO production phase.

FIG. 9 shows the ability of other rare earth metals to activate or repress the pMxaF promoter as measured by mCherry levels. Cerium (Ce) and Lanthanum (La) were able to repress the pMxaF promoter whether or not they were present in the media during pre-culture or added after the pre-culture period. Further, dysprosium (Dy) or erbium (Er) were not able to repress the pMxaF promoter at any time.

FIG. 10 shows that a titration of different levels of cerium on the effect of its ability to repress the pMxaF promoter. Full repression of the pMxaF promoter occur at approximately 70 μM of cerium. At this level, repression is similar to that of 10 or 35 μM of lanthanum.

FIG. 11 shows the ability of different rare earth metals to activate or repress the pMxaF promoter as measured by mCherry. Lanthanum (La), praseodymium (Pr), and neodymium (Nd) repressed the pMxaF promoter. Additionally, europium (Eu), gadolinium (Gd), terbium (Tb), thulium (Tm), and ytterbium (Yb), did not repress the pMxaF promoter. In fact, these later rare earth metals activated the expression of the pMxaF promoter.

FIG. 12 shows the ability of various rare earth metals at a concentration of 10 μM, 35 μM, 70 μM, or 140 μM to activate or repress the pMxaF promoter as measured by mCherry. Scandium (Sc), yttrium (Y), samarium (Sm), europium (Eu), gadolinium (Gd), terbium (Tb), dysprosium (Dy), holmium (Ho), erbium (Er), thulium (Tm), and ytterbium (Yb) minimally activated or repressed the pMxaF promoter at 10 μM, 35 μM, 70 μM, or 140 μM. However, lanthanum (La), cerium (Ce), praseodymium (Pr), and neodymium (Nd) significantly repressed the expression of the pMxaF promoter at all concentrations. Maximal repression was observed starting at 35 μM for lanthanum, 70 μM for cerium, and 140 μM for praseodymium and neodymium.

### DETAILED DESCRIPTION OF THE INVENTION

Generally, fermentation using a microorganism that produces a valued chemical, whether naturally occurring or genetically modified, progresses in two stages. The first stage is known as the growth stage. In this growth stage, the microorganism produces minimal valued chemical or in some cases, does not produce any valued chemical at all. The great majority or all of the microbe's energy is devoted to the cellular reproduction mechanism, e.g. cell division. In this case, very few if any, genes that are used to produce the valued chemical are expressed or should be expressed. After enough cells are produced and are matured, the second stage is turned on. This second stage is the stage when the valued chemical is produced. In some cases, the turning on of the second stage naturally occurs after the microorganism is within conditions that promotes the production of the valued chemical. For genetically modified microorganisms, the turning on of the second stage can be “forcefully” induced as-needed and expression of genes can be regulated by a molecular switch.

As discussed, aspects of the invention include molecular switches that can be used to repress or activate the expression of genes within a microorganism. In some cases, the microorganism is genetically modified to produce desired valued chemicals that it would not normally produce or produce naturally producing chemicals but at an increased level. Also disclosed are the microorganisms that comprise the molecular switches, methods of using the microorganisms and/or the molecular switches, as well as making the microorganism and/or molecular switches.

Before the present invention is described in greater detail, it is to be understood that this invention is not limited to particular cases described, as such can, of course, vary. It is also to be understood that the terminology used herein is for the purpose of describing particular cases only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

### I. Definitions

The term “switch” and its grammatical equivalents as used herein can mean a regulatory unit of a gene or genes that is capable of responding to a particular stimulus to either induce or repress expression. For example, switches can include regulatory units that respond to sugar (e.g., arabinose) or rare earth metals (e.g., lanthanum).

The term “rare earth metal” and its grammatical equivalent as used herein can mean any chemical element defined as such by the IUPAC, which can include but is not limited to cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), scandium (Sc), terbium (Tb), thulium (Tm), ytterbium (Yb), and yttrium (Y).

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical disclosed throughout can be “about” that numerical value even without specifically mentioning the term “about.”

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “genetic modification” or “genetically modified” and their grammatical equivalents as used herein can refer to one or more alterations of a nucleic acid, e.g., the nucleic acid within a microorganism's genome. For example, genetic modification can refer to alterations, additions, and/or deletion of nucleic acid (e.g., whole genes or fragments of genes).

The term “disrupting” and its grammatical equivalents as used herein can refer to a process of altering a gene, e.g., by deletion, insertion, mutation, rearrangement, or any combination thereof. For example, a gene can be disrupted by knockout. Disrupting a gene can be partially reducing or completely suppressing expression (e.g., mRNA and/or protein expression) of the gene. Disrupting can also include inhibitory technology, such as shRNA, siRNA, microRNA, dominant negative, or any other means to inhibit functionality or expression of a gene or protein.

The term “gene editing” and its grammatical equivalents as used herein can refer to genetic engineering in which one or more nucleotides are inserted, replaced, or removed from a genome. For example, gene editing can be performed using a nuclease (e.g., a natural-existing nuclease or an artificially engineered nuclease).

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.”

The term “promoter” and its grammatical equivalents as used herein can refer to a nucleic acid sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity. Some examples of promoters that can be used include but are not limited to pXoxF, pMxaF, pTRC, J23111, J12100, J23102, pBAD, J23110, lacO, J23116, J23106, J23105, J23108, J23107, J23115, J23114, J23118, J23104, J23101, J23119, and uMCA3034.

The term “operably linked” and its grammatical equivalents as used herein can refer to the association of nucleic acid sequences on a single polynucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “codon optimized” and its grammatical equivalents as used herein insofar as it refers to genes or coding regions of nucleic acid molecules (or open reading frames) for transformation of various hosts, can refer to the alteration of codons in the gene or coding regions of the nucleic acid molecules to reflect the typical codon usage of the host organism without altering the polypeptide encoded by the DNA.

The term “open reading frame” (“ORF”) and its grammatical equivalents as used herein can refer to a polynucleic acid or nucleic acid sequence (whether naturally occurring, non-naturally occurring, or synthetic) comprising an uninterrupted reading frame consisting of (i) an initiation codon, (ii) a series of two (2) of more codons representing amino acids, and (iii) a termination codon, the ORF being read (or translated) in the 5′ to 3′ direction.

The term “operon” and its grammatical equivalents as used herein can refer to two or more genes which are transcribed as a single transcriptional unit from a common promoter. In certain cases, the genes, polynucleotides or ORFs comprising the operon are contiguous. It is understood that transcription of an entire operon can be modified (i.e., increased, decreased, or eliminated) by modifying the common promoter. Alternatively, any gene, polynucleotide or ORF, or any combination thereof in an operon can be modified to alter the function or activity of the encoded polypeptide. The modification can result in an increase or a decrease in the activity or function of the encoded polypeptide. Further, the modification can impart new activities on the encoded polypeptide.

The term “vector” and its grammatical equivalents as used herein can refer to any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes”, that is, that replicate autonomously or can integrate into a chromosome of a host microorganism. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

As used herein, the terms “C1 carbon,” “C1-carbon substrates” and their grammatical equivalents can refer to any organic compound that contains a single carbon atom. Examples include, but are not limited to, carbon monoxide (CO), methane (CH₄), and carbon dioxide (CO₂).

As used herein, the term “fermentation” or “fermentation process,” and its grammatical equivalents, can be a process in which a host cell is cultivated in a culture medium containing raw materials, such as feedstock and nutrients, wherein the cell converts raw materials, such as a feedstock, into desirable end products, such as multicarbon products.

As used herein, the term “substantially pure” and its grammatical equivalents can refer to a particular substance that does not contain a majority of another substance. For example, “substantially pure product” can mean at least 90% of that product. In some instances, “substantially pure product” can mean at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, 99.999%, or 99.9999% of that product. For example, substantially pure product can mean at least 70% of the product. In some cases, substantially pure product can mean at least 75% of the product. In some cases, substantially pure product can mean at least 80% of the product. In some cases, substantially pure product can mean at least 85% of the product. In some cases, substantially pure product can mean at least 90% of the product. In some cases, substantially pure product can mean at least 91% of the product. In some cases, substantially pure product can mean at least 92% of the product. In some cases, substantially pure product can mean at least 93% of the product. In some cases, substantially pure product can mean at least 94% of the product. In some cases, substantially pure product can mean at least 95% of the product. In some cases, substantially pure product can mean at least 96% of the product. In some cases, substantially pure product can mean at least 97% of the product. In some cases, substantially pure product can mean at least 98% of the product. In some cases, substantially pure product can mean at least 99% of the product.

As used herein, the term “heterologous” and its grammatical equivalents can mean derived from a different species. For example, a “heterologous gene” can mean a gene that is from a species different than the reference species. For example, a methanotroph comprising a “heterologous gene” comprises a gene that is not from the same methanotroph. The gene can be from a different microorganism such as yeast or from a different species such as a different methanotroph species.

As used herein, the term “substantially similar” and its grammatical equivalents, when used in reference to the similarity between a sequence and a reference sequence, means that the sequences are at least 50% (but not 100%) identical. In some cases, the sequences are 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, or 99.9999% identical. In some cases, the term substantially similar refers to a sequence that is at least 50% identical. In some instances, the term substantially similar refers to a sequence that is 55% identical. In some instances, the term substantially similar refers to a sequence that is 60% identical. In some instances, the term substantially similar refers to a sequence that is 65% identical. In some instances, the term substantially similar refers to a sequence that is 70% identical. In some instances, the term substantially similar refers to a sequence that is 75% identical. In some instances, the term substantially similar refers to a sequence that is 80% identical. In other instances, the term substantially similar refers to a sequence that is 81% identical. In other instances, the term substantially similar refers to a sequence that is 82% identical. In other instances, the term substantially similar refers to a sequence that is 83% identical. In other instances, the term substantially similar refers to a sequence that is 84% identical. In other instances, the term substantially similar refers to a sequence that is 85% identical. In other instances, the term substantially similar refers to a sequence that is 86% identical. In other instances, the term substantially similar refers to a sequence that is 87% identical. In other instances, the term substantially similar refers to a sequence that is 88% identical. In other instances, the term substantially similar refers to a sequence that is 89% identical. In some instances, the term substantially similar refers to a sequence that is 90% identical. In some instances, the term substantially similar refers to a sequence that is 91% identical. In some instances, the term substantially similar refers to a sequence that is 92% identical. In some instances, the term substantially similar refers to a sequence that is 93% identical. In some instances, the term substantially similar refers to a sequence that is 94% identical. In some instances, the term substantially similar refers to a sequence that is 95% identical. In some instances, the term substantially similar refers to a sequence that is 96% identical. In some instances, the term substantially similar refers to a sequence that is 97% identical. In some instances, the term substantially similar refers to a sequence that is 98% identical. In some instances, the term substantially similar refers to a sequence that is 99% identical. In some instances, the term substantially similar refers to a sequence that is 100% identical. To determine the percentage of identity between two sequences, the two sequences are aligned, using, for example, the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids/nucleotides is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, c.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Molec. Biol., 1990:215:403). A particularly preferred method for determining the percentage identity between two polypeptides involves the Clustal W algorithm (Thompson, J D, Higgines, D G and Gibson T J, 1994, Nucleic Acid Res 22(22): 4673-4680 together with the BLOSUM 62 scoring matrix (Henikoff S & Henikoff, J G, 1992, Proc. Natl. Acad. Sci. USA 89: 10915-10919) using a gap opening penalty of 10 and a gap extension penalty of 0.1, so that the highest order match obtained between two sequences wherein at least 50% of the total length of one of the two sequences is involved in the alignment.

As will be apparent to those of skill in the art upon reading this disclosure, each of the individual cases described and illustrated herein has discrete components and features which may be readily separated from or combined with the features of any of the other several cases without departing from the scope or spirit of the present invention. Any recited method can be carried out in the order of events recited or in any other order which is logically possible.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

### II. Genetically Modified Microorganisms and Methods of Making the Same

The present disclosure is directed, in part, to genetically modified microorganisms that have dramatically improved ability to produce desired chemicals compared to its wild-type counterpart. For example, the genetically modified microorganisms can produce valuable chemicals, including but not limited to, 2,3-butanediol (“2,3-BDO”); 1,4-butanediol (“1,4-BDO”); isobutyraldehyde; isobutanol; 1-butanol; n-butanol; ethanol; fatty (or aliphatic long chain) alcohols; and fatty acid methyl esters. In some cases, the fermentation titers of the desired valuable chemicals are orders of magnitude higher than what could be normally produced. In some instances, the microorganisms that do not naturally produce a specific valuable chemical have been genetically modified to synthesize the valuable chemical. The use of these molecular switches allow for direct control of gene expression at a given time. This control of gene expression by using molecular switches can lead to increased levels of valuable chemical titer production compared to microorganism that do not have these molecular switches.

### Microorganisms

In some cases, the microorganisms can use C1 carbon substrates, such as, CO, CO₂, and CH₄, to synthesize a desired end product (e.g., a multicarbon product). This, however, does not mean that these microorganisms use solely C1 carbons. Some of the microorganisms can be made to utilize additional carbon substrates, including carbon substrates that the microorganism naturally uses. For example, if the microorganism naturally uses sugar for carbon substrates, this microorganism can be made to utilize a different carbon source such as a C1 carbon.

The microorganisms can be a prokaryote or a eukaryote. In some cases, for example, the microorganisms can be bacteria, yeast, or algae.

Microorganisms that can convert C1 carbon substrates into desired products include those capable of using natural gas as a carbon substrate. For example, the microorganism can use methane contained within the natural gas a as a carbon source to make such desired products. Such microorganisms can include methanotrophs. Methanotrophs that can be particularly useful include those from the genera Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, Methyloacidophilum, or any combinations thereof. In some cases, the methanotroph is from the genus Methylococcus. In one instance, the methanotroph can be a methanotroph from the species Methylococcus capsulatus.

Some microorganisms are capable of using CO₂ as a substrate. Such microorganisms include methanogens. Microorganisms that are capable of using CO₂ as a substrate can contain chlorophyll. Examples thereof include algae and cyanobacteria.

Some microorganisms are capable of using CO as a substrate. Examples include anaerobic microorganisms such as Clostridium. These microorganism can be genetically modified so as to make substantial amounts of desired valued chemical products.

### Nucleic Acids Encoding for Enzymes

Certain enzymes can be used to generate valued chemical products. Some valued chemical products can include, but are not limited to, 2,3-butanediol (“2,3-BDO”); 1,4-butanediol (“1,4-BDO”); isobutyraldehyde; isobutanol; 1-butanol (aka n-butanol); ethanol; fatty (or aliphatic long chain) alcohols; and fatty acid methyl ester. In some cases, the polynucleotide of the promoters encoding these enzymes can be altered so that they are responsive to the substances described herein (e.g., sugars and rare earth metals) and therefore can function as a molecular switch.

### 2,3-butanediol (“2,3-BDO”)

In some instances, polynucleotides encoding enzymes of the 2,3-BDO pathway can be used. For example, the microorganism can contain (either endogenously or heterologous) one or more polynucleotides encoding for an acetolactate synthase (AlsS), alpha-acetolactate decarboxylase (budA), and/or acetoin reductase. One or more of the polynucleotides can be native to the microorganism. In some cases, one or more of the polynucleotides can be heterologous to the microorganism.

In some cases, the acetolactate synthase can be encoded by a polynucleotide that is substantially similar to any one of SEQ ID NOs: 2, 4, or 6.

In some cases, the alpha-acetolactate decarboxylase can be encoded by a polynucleotide that is substantially similar to SEQ ID NO: 8 or 10.

In some cases, the acetoin reductase can be encoded by a polynucleotide that is substantially similar to any one of SEQ ID NOs: 12, 14, or 16. In some cases, the acetoin reductase can be NADPH-dependent. In some cases, the acetoin reductase can be NADH-dependent.

In order to engineer a microorganism that can produce 2,3-BDO, one or more genes (e.g., heterologous genes) can be transformed or transfected into the microorganism, either transiently or stably. In some cases, one or more of these genes can be episomally expressed. In some cases, one of more of these genes can be integrated into the genome of the microorganism. In some cases, one or more of these genes be can be episomally expressed whereas one or more of these genes can be integrated into the genome of the microorganism. In some cases, the engineered microorganism can utilize one or more of the following enzymes: (i) acctolactate synthase, (ii) alpha-acetolactate decarboxylase, and/or (iii) acetoin reductase. Acctolactate synthase (encoded by the gene AlsS) converts two molecules of pyruvate into 2-acetolactate. Alpha-acetolactate decarboxylase (encoded by the gene BudA) converts 2-acetolactate into acetoin. Acetoin reductase (encoded by the gene ButA) converts acetoin into 2,3-BDO using NADPH or NADH as a reduced cofactor. Acetoin reductases that use NADPH as a cofactor are referred to as “NADPH-dependent acetoin reductase(s).” Acetoin reductases that use NADH as a cofactor are referred to as “NADH-dependent acetoin reductase(s).”

Described herein are methods of making microorganisms used to make 2,3-BDO from a C1 carbon (e.g., methane). In some cases, the microorganism herein can be transformed with a gene encoding one or more of the following enzymes: (i) acetoin reductase (NADPH-dependent and/or NADH-dependent); (ii) alpha-acetolactate decarboxylase; and/or (iii) acetolactate synthase (AlsS). For example, the microorganism can be transformed with a gene encoding a NADPH- or NADH-dependent acetoin reductase. As another example, the microorganism can be transformed with a gene encoding an alpha-acetolactate decarboxylase. As yet another example, the microorganism can be transformed with a gene encoding an acetolactate synthase. These genes can be heterologous to the microorganism. In some cases, these genes can be episomally expressed, or integrated into the genome of the microorganism (e.g., through the use of an integration vector), or any combination of thereof.

In some cases, the microorganism can be transformed with two or more genes such as those encoding NADPH- and/or NADH-dependent acetoin reductase and alpha-acetolactate decarboxylase. As another example, the microorganism can also be transformed with genes encoding NADPH- or NADH-dependent acetoin reductase and acetolactate synthase. As yet another example, the microorganism can be transformed with genes encoding an alpha-acetolactate decarboxylase and acetolactate synthase. One or more of the genes can be heterologous to the microorganism. In some cases, these genes can be episomally expressed, or integrated into the genome of the microorganism, or any combination of thereof.

In some cases, the microorganism can be transformed with at least three or more genes such as those encoding NADPH- and/or NADH-dependent acetoin reductase, alpha-acetolactate decarboxylase, and acetolactate synthase. One or more of the genes can be heterologous to the microorganism. In some cases, these genes can be episomally expressed, or integrated into the genome of the microorganism, or any combination of thereof.

The gene encoding the NADPH-dependent acetoin reductase can be from a bacteria (e.g., a gram positive or gram negative bacterium). The bacterium can be from the genus Clostridium, for example, Clostridium autoethanogenum.

The NADPH-dependent acetoin reductase can comprise an amino acid sequence that is substantially similar to SEQ ID NO: 11. For example, the NADPH-dependent acetoin reductase may comprise an amino acid sequence that is at least 65%, 70%, 75%, 80%, 85%, 90%, 91%, 92%, 93%, 94%, 95%, 96%, 97%, 98%, 99%, 99.9%, 99.99%, 99.999%, or 99.9999% identical to SEQ ID NO: 11. In some cases, the NADPH-dependent acetoin reductase can comprise an amino acid sequence that is at least 60% identical to SEQ ID NO: 11. In some cases, the NADPH-dependent acetoin reductase can comprise an amino acid sequence that is at least 65% identical to SEQ ID NO: 11. In some cases, the NADPH-dependent acetoin reductase can comprise an amino acid sequence that is at least 70% identical to SEQ ID NO: 11. In some cases, the NADPH-dependent acetoin reductase can comprise an amino acid sequence that is at least 75% identical to SEQ ID NO: 11. In some cases, the NADPH-dependent acetoin reductase can comprise an amino acid sequence that is at least 80% identical to SEQ ID NO: 11. In some cases, the NADPH-dependent acetoin reductase can comprise an amino acid sequence that is at least 85% identical to SEQ ID NO: 11. In some cases, the NADPH-dependent acetoin reductase can comprise an amino acid sequence that is at least 90% identical to SEQ ID NO: 11. In some cases, the NADPH-dependent acetoin reductase can comprise an amino acid sequence that is at least 91% identical to SEQ ID NO: 11. In some cases, the NADPH-dependent acetoin reductase can comprise an amino acid sequence that is at least 92% identical to SEQ ID NO: 11. In some cases, the NADPH-dependent acetoin reductase can comprise an amino acid sequence that is at least 93% identical to SEQ ID. NO: 11. In some cases, the NADPH-dependent acetoin reductase can comprise an amino acid sequence that is at least 94% identical to SEQ ID NO: 11. In some cases, the NADPH-dependent acetoin reductase can comprise an amino acid sequence that is at least 95% identical to SEQ ID NO: 11. In some cases, the NADPH-dependent acetoin reductase can comprise an amino acid sequence that is at least 96% identical to SEQ ID NO: 11. In some cases, the NADPH-dependent acetoin reductase can comprise an amino acid sequence that is at least 97% identical to SEQ ID NO: 11. In some cases, the NADPH-dependent acetoin reductase can comprise an amino acid sequence that is at least 98% identical to SEQ ID NO: 11. In some cases, the NADPH-dependent acetoin reductase can comprise an amino acid sequence that is at least 99% identical to SEQ ID NO: 11. In some cases, the NADPH-dependent acetoin reductase can comprise an amino acid sequence that is SEQ ID NO: 11. In some cases, the NADPH-dependent acetoin reductase comprise the amino acid sequence SEQ ID NO: 11.

The gene encoding the NADH-dependent acetoin reductase can be from a bacteria (e.g., a gram positive or gram negative bacteria). Examples include those from the genus Bacillus, for example Bacillus subtilis. The bacteria can be from the genus Paenibacillus, for example Paenibacillus polymyxa.

In some cases, the NADH-dependent acetoin reductase can comprise an amino acid sequence that is substantially similar to SEQ ID NO: 13 or 15. For example, the NADH-dependent acetoin reductase may comprises an amino acid sequence that is at least 60%, 65%, 70%, 75%, 80%, 85%, 90%, 91%, 92%, 93%, 94%, 95%, 96%, 97%, 98%, 99%, 99.9%, 99.99%, 99.999%, or 99.9999% identical to SEQ ID NO: 13 or 15. In some cases, the NADH-dependent acetoin reductase can comprise an amino acid sequence that is at least 60% identical to SEQ ID NO: 13 or 15. In some cases, the NADH-dependent acetoin reductase can comprise an amino acid sequence that is at least 65% identical to SEQ ID NO: 13 or 15. In some cases, the NADH-dependent acetoin reductase can comprise an amino acid sequence that is at least 70% identical to SEQ ID NO: 13 or 15. In some cases, the NADH-dependent acetoin reductase can comprise an amino acid sequence that is at least 75% identical to SEQ ID NO: 13 or 15. In some cases, the NADH-dependent acetoin reductase can comprise an amino acid sequence that is at least 80% identical to SEQ ID NO: 13 or 15. In some cases, the NADH-dependent acetoin reductase can comprise an amino acid sequence that is at least 85% identical to SEQ ID NO: 13 or 15. In some cases, the NADH-dependent acetoin reductase can comprise an amino acid sequence that is at least 90% identical to SEQ ID NO: 13 or 15. In some cases, the NADH-dependent acetoin reductase can comprise an amino acid sequence that is at least 91% identical to SEQ ID NO: 13 or 15. In some cases, the NADH-dependent acetoin reductase can comprise an amino acid sequence that is at least 92% identical to SEQ ID NO: 13 or 15. In some cases, the NADH-dependent acetoin reductase can comprise an amino acid sequence that is at least 93% identical to SEQ ID NO: 13 or 15. In some cases, the NADH-dependent acetoin reductase can comprise an amino acid sequence that is at least 94% identical to SEQ ID NO: 13 or 15. In some cases, the NADH-dependent acetoin reductase can comprise an amino acid sequence that is at least 95% identical to SEQ ID NO: 13 or 15. In some cases, the NADH-dependent acetoin reductase can comprise an amino acid sequence that is at least 96% identical to SEQ ID NO: 13 or 15. In some cases, the NADH-dependent acetoin reductase can comprise an amino acid sequence that is at least 97% identical to SEQ ID NO: 13 or 15. In some cases, the NADH-dependent acetoin reductase can comprise an amino acid sequence that is at least 98% identical to SEQ ID NO: 13 or 15. In some cases, the NADH-dependent acetoin reductase can comprise an amino acid sequence that is at least 99% identical to SEQ ID NO: 13 or 15. In some cases, the NADH-dependent acetoin reductase can comprise the amino acid sequence SEQ ID NO: 13 or 15.

The gene encoding the alpha-acetolactate decarboxylase (budA) can be from a bacterium (e.g., a gram positive bacteria or gram negative bacterium). Examples include those from the genus Clostridium, for example Clostridium autoethanogenum. Other examples include those from the genus Klebsiella, for example Klebsiella pneumoniae.

In some cases, the alpha-acetolactate decarboxylase can comprise an amino acid sequence that is substantially similar to SEQ ID NO: 7 or 9. For example, the alpha-acetolactate decarboxylase can comprise an amino acid sequence that is at least 60% 65%, 70%, 75%, 80%, 85%, 90%, 91%, 92%, 93%, 94%, 95%, 96%, 97%, 98%, 99%, 99.9%, 99.99%, 99.999%, or 99.9999% identical to SEQ ID NO: 7 or 9. In some cases, the alpha-acetolactate decarboxylase can comprise an amino acid sequence that is at least 60% identical to SEQ ID NO: 7 or 9. For example, the alpha-acetolactate decarboxylase can comprise an amino acid sequence that is at least 65% identical to SEQ ID NO: 7 or 9. In some cases, the alpha-acetolactate decarboxylase can comprise an amino acid sequence that is at least 70% identical to SEQ ID NO: 7 or 9. For example, the alpha-acetolactate decarboxylase can comprise an amino acid sequence that is at least 75% identical to SEQ ID NO: 7 or 9. In some cases, the alpha-acetolactate decarboxylase can comprise an amino acid sequence that is at least 80% identical to SEQ ID NO: 7 or 9. For example, the alpha-acetolactate decarboxylase can comprise an amino acid sequence that is at least 85% identical to SEQ ID NO: 7 or 9. In some cases, the alpha-acetolactate decarboxylase can comprise an amino acid sequence that is at least 90% identical to SEQ ID NO: 7 or 9. In some cases, the alpha-acetolactate decarboxylase can comprise an amino acid sequence that is at least 91% identical to SEQ ID NO: 7 or 9. In some cases, the alpha-acetolactate decarboxylase can comprise an amino acid sequence that is at least 92% identical to SEQ ID NO: 7 or 9. In some cases, the alpha-acetolactate decarboxylase can comprise an amino acid sequence that is at least 93% identical to SEQ ID NO: 7 or 9. In some cases, the alpha-acetolactate decarboxylase can comprise an amino acid sequence that is at least 94% identical to SEQ ID NO: 7 or 9. In some cases, the alpha-acetolactate decarboxylase can comprise an amino acid sequence that is at least 95% identical to SEQ ID NO: 7 or 9. In some cases, the alpha-acetolactate decarboxylase can comprise an amino acid sequence that is at least 96% identical to SEQ ID NO: 7 or 9. In some cases, the alpha-acetolactate decarboxylase can comprise an amino acid sequence that is at least 97% identical to SEQ ID NO: 7 or 9. In some cases, the alpha-acetolactate decarboxylase can comprise an amino acid sequence that is at least 98% identical to SEQ ID NO: 7 or 9. In some cases, the alpha-acetolactate decarboxylase can comprise an amino acid sequence that is at least 99% identical to SEQ ID NO: 7 or 9. In some cases, the alpha-acetolactate decarboxylase can comprise an amino acid sequence that is identical to SEQ ID NO: 7 or 9. In some cases, the alpha-acetolactate decarboxylase can comprise the amino acid sequence SEQ ID NO: 7 or 9.

The gene encoding the acetolactate synthase (AlsS) can be from a bacterium (e.g., a gram positive bacterium). Examples include those from the genus Clostridium, for example Clostridium autoethanogenum. Other examples include those from the genus Bacillus, for example Bacillus subtilis. Additional species example include Bacillus licheniformis.

In some cases, the acetolactate synthase can comprise an amino acid sequence that is substantially similar to anyone of SEQ ID NOs: 1, 3, or 5. In some cases, the acetolactate synthase can comprise an amino acid sequence that is at least 60% 65%, 70%, 75%, 80%, 85%, 90%, 91%, 92%, 93%, 94%, 95%, 96%, 97%, 98%, 99%, 99.9%, 99.99%, 99.999%, or 99.9999% identical to anyone of SEQ ID NOs: 1, 3, or 5. In some cases, the acetolactate synthase can comprise an amino acid sequence that is at least 60% identical to anyone of SEQ ID NOs: 1, 3, or 5. In some cases, the acetolactate synthase can comprise an amino acid sequence that is at least 65% identical to anyone of SEQ ID NOs: 1, 3, or 5. In some cases, the acetolactate synthase can comprise an amino acid sequence that is at least 70% identical to anyone of SEQ ID NOs: 1, 3, or 5. In some cases, the acetolactate synthase can comprise an amino acid sequence that is at least 75% identical to anyone of SEQ ID NOs: 1, 3, or 5. In some cases, the acetolactate synthase can comprise an amino acid sequence that is at least 80% identical to anyone of SEQ ID NOs: 1, 3, or 5. In some cases, the acetolactate synthase can comprise an amino acid sequence that is at least 85% identical to anyone of SEQ ID NOs: 1, 3, or 5. In some cases, the acetolactate synthase can comprise an amino acid sequence that is at least 90% identical to anyone of SEQ ID NOs: 1, 3, or 5. In some cases, the acetolactate synthase can comprise an amino acid sequence that is at least 91% identical to anyone of SEQ ID NOs: 1, 3, or 5. In some cases, the acetolactate synthase can comprise an amino acid sequence that is at least 92% identical to anyone of SEQ ID NOs: 1, 3, or 5. In some cases, the acetolactate synthase can comprise an amino acid sequence that is at least 93% identical to anyone of SEQ ID NOs: 1, 3, or 5. In some cases, the acetolactate synthase can comprise an amino acid sequence that is at least 94% identical to anyone of SEQ ID NOs: 1, 3, or 5. In some cases, the acetolactate synthase can comprise an amino acid sequence that is at least 95% identical to anyone of SEQ ID NOs: 1, 3, or 5. In some cases, the acetolactate synthase can comprise an amino acid sequence that is at least 96% identical to anyone of SEQ ID NOs: 1, 3, or 5. In some cases, the acetolactate synthase can comprise an amino acid sequence that is at least 97% identical to anyone of SEQ ID NOs: 1, 3, or 5. In some cases, the acetolactate synthase can comprise an amino acid sequence that is at least 98% identical to anyone of SEQ ID NOs: 1, 3, or 5. In some cases, the acetolactate synthase can comprise an amino acid sequence that is at least 99% identical to anyone of SEQ ID NOs: 1, 3, or 5. In some cases, the acetolactate synthase can comprise an amino acid sequence that is identical to anyone of SEQ ID NOs: 1, 3, or 5.

In some cases, additional enzymes can be provided to the microorganism to yield other desired end products by fermentation.

In some cases, the amino acid sequence can be optimized based on the microorganism in which the genes will be provided or the enzymes will be expressed. In such cases, conservative amino acids substitutions can be made based on whether the microorganism typically uses a specific amino acid or how much of that particular amino acid is available for use within the microorganism.

In some cases, the codons can be optimized based on the microorganism in which the genes will be provided or the enzymes will be expressed. In some cases, one or more the nucleotide sequences encoding for one or more of the enzymes in the 2,3-BDO pathway can be driven by a molecular switch. In some cases, the molecular switch can be turned on or off by a chemical substance as described throughout. In some cases, the chemical substance can be a rare earth metal. In some cases, the rare earth metal can be lanthanum.

### 1,4-butanediol (“1,4-BDO”)

In some instances, polynucleotides encoding enzymes of the 1,4-BDO pathway can be used. For example, the microorganism can contain (either endogenously or heterologous) one or more polynucleotides encoding for a succinyl-CoA synthetase (sucC), CoA-dependent succinate semialdehyde dehydrogenase (sucD), 4-hydroxybutyrate dehydrogenase (4hbD), 4-hydroxybutyryl-CoA transferase (cat2), aldehyde dehydrogenase (ald), and/or alcohol dehydrogenase (adh).

In some cases, the 4-hydroxybutyrate dehydrogenase (4hbD) can be encoded by a polynucleotide that is substantially similar to SEQ ID NO. 22. In some cases, the 4-hydroxybutyrate dehydrogenase can be encoded by an amino acid sequence that is substantially similar SEQ ID NO: 21.

In some cases, the 4-hydroxybutyrate CoA transferase (Cat2) can be encoded by a polynucleotide that is substantially similar to SEQ ID NO: 24. In some cases, the 4-hydroxybutyrate CoA transferase can be encoded by an amino acid sequence that is substantially similar SEQ ID NO: 23.

In some cases, the aldehyde dehydrogenase gene can be encoded by a polynucleotide that is substantially similar to SEQ ID NO: 26. In some cases, the aldehyde dehydrogenase can be encoded by an amino acid sequence that is substantially similar SEQ ID NO: 25.

In some cases, the alcohol dehydrogenase can be encoded by a polynucleotide that is substantially similar to SEQ ID NO: 28. In some cases, the alcohol dehydrogenase can be encoded by an amino acid sequence that is substantially similar SEQ ID NO: 27.

In some cases, the succinyl CoA synthetase beta subunit (sucC) can be encoded by a polynucleotide that is substantially similar SEQ ID NO: 18. In some cases, the succinyl CoA synthetase beta subunit can be encoded by an amino acid sequence that is substantially similar SEQ ID NO: 17.

In some cases, the CoA-dependent succinate semialdehyde dehydrogenase (sucD) can be encoded by a polynucleotide that is substantially similar to SEQ ID NO: 20. In some cases, the CoA-dependent succinate semialdehyde dehydrogenase can be encoded by an amino acid sequence that is substantially similar SEQ ID NO: 19.

In some cases, the codons can be optimized based on the microorganism in which the genes will be provided or the enzymes will be expressed. In some cases, one or more the nucleotide sequences encoding for one or more of the enzymes in the 1,4-BDO pathway can be driven by a molecular switch. In some cases, the molecular switch can be turned on or off by a chemical substance as described throughout. In some cases, the chemical substance can be a rare earth metal. In some cases, the rare earth metal can be lanthanum.

### Isobutyraldehyde

In some instances, polynucleotides encoding enzymes of the isobutyraldehyde pathway can be used. For example, the microorganism can comprise one or more polynucleotides encoding for one or more enzymes from the EC 2.2.1.6, EC 1.1.1.86, EC 4.2.1.9, and/or EC 4.1.1.72. In some cases, the microorganism can comprises one or more polynucleotides (either endogenously or heterologous) encoding for an acetolactate synthase (AlsS); ketol-acid reductoisomerase (KARI); dihydroxy-acid dehydratase (DHAD); and/or 2-keto acid decarboxylase (KDC). One or more of the polynucleotides can be native to the microorganism. In some cases, one or more of the polynucleotides can be heterologous to the microorganism.

In some cases, the AlsS can be encoded by a polynucleotide that is substantially similar to SEQ ID NO: 30. In some cases, the AlsS can be encoded by an amino acid sequence that is substantially similar to SEQ ID NO: 29. In some cases, the ketol-acid reductoisomerase can be encoded by a polynucleotide that is substantially similar to SEQ ID NO: 32. In some cases, the ketol-acid reductoisomerase can be encoded by an amino acid sequence that is substantially similar to SEQ ID NO: 31. In some cases, the dihydroxy-acid dehydratase can be encoded by a polynucleotide that is substantially similar to SEQ ID NO: 34. In some cases, the dihydroxy-acid dehydratase can be encoded by an amino acid sequence that is substantially similar to SEQ ID NO: 33. In some cases, the KDC can be encoded by a polynucleotide that is substantially similar to any one of SEQ ID NOs: 36, 38, 40, 42, 44, 46 or 48. In some cases, the KDC can be encoded by an amino acid sequence that is substantially similar to any one of SEQ ID NOs: 35, 37 39, 41, 43, 45, or 47.

In some cases, the codons can be optimized based on the microorganism in which the genes will be provided or the enzymes will be expressed. In some cases, one or more the nucleotide sequences encoding for one or more of the enzymes in the isobutyraldehyde pathway can be driven by a molecular switch. In some cases, the molecular switch can be turned on or off by a chemical substance as described throughout. In some cases, the chemical substance can be a rare earth metal. In some cases, the rare earth metal can be lanthanum.

### Isobutanol

In some instances, polynucleotides encoding enzymes of the isobutanol pathway can be used. For example, the microorganism can comprise one or more polynucleotides encoding for one or more enzymes from the EC 2.2.1.6, EC 1.1.1.86, EC 4.2.1.9, EC 4.1.1.72 and/or EC 1.1.1.1. In some cases, the microorganism can comprises one or more polynucleotides (cither endogenously or heterologous) encoding for an acetolactate synthase (AlsS); ketol-acid reductoisomerase (KARI); dihydroxy-acid dehydratase (DHAD); 2-keto acid decarboxylase (KDC), and/or alcohol dehydrogenase (ADH). One or more of the polynucleotides can be native to the microorganism. In some cases, one or more of the polynucleotides can be heterologous to the microorganism.

In some cases, the AlsS can be encoded by a polynucleotide that is substantially similar to SEQ ID NO: 30. In some cases, the AlsS can be encoded by an amino acid sequence that is substantially similar to SEQ ID NO: 29. In some cases, the ketol-acid reductoisomerase can be encoded by a polynucleotide that is substantially similar to SEQ ID NO: 32. In some cases, the ketol-acid reductoisomerase can be encoded by an amino acid sequence that is substantially similar to SEQ ID NO: 31. In some cases, the dihydroxy-acid dehydratase can be encoded by a polynucleotide that is substantially similar to SEQ ID NO: 34. In some cases, the dihydroxy-acid dehydratase can be encoded by an amino acid sequence that is substantially similar to SEQ ID NO: 33. In some cases, the KDC can be encoded by a polynucleotide that is substantially similar to any one of SEQ ID NOs: 36, 38, 40, 42, 44, 46 or 48. In some cases, the KDC can be encoded by an amino acid sequence that is substantially similar to any one of SEQ ID NOs: 35, 37 39, 41, 43, 45, or 47. In some cases, the ADH can be encoded by a polynucleotide that is substantially similar to any one of SEQ ID NOs: 50, 52, 54, 56 or 58. In some cases, the ADH can be encoded by an amino acid that is substantially similar to any one of SEQ ID NOs: 49, 51, 53, 55 or 57.

In some cases, the codons can be optimized based on the microorganism in which the genes will be provided or the enzymes will be expressed. In some cases, one or more the nucleotide sequences encoding for one or more of the enzymes in the isobutanol pathway can be driven by a molecular switch. In some cases, the molecular switch can be turned on or off by a chemical substance as described throughout. In some cases, the chemical substance can be a rare earth metal. In some cases, the rare earth metal can be lanthanum.

### 1-butanol (aka n-butanol)

In some cases, polynucleotides encoding for enzymes of the 1-butanol pathway can be used. For example, the microorganism can contain (either endogenously or heterologous) one or more polynucleotides encoding for an enzyme from the Enzyme Class EC 4.3.1.19, EC 2.3.3.6, EC 4.2.1.33, EC 4.1.1.72, and/or EC 1.1.1.1. In some cases, the polynucleotides encode for one or more polypeptides that is L-threonine ammonia lyase, 2-ethylmalate synthase, isopropylmalate isomerase, 2-ketoacid decarboxylase (KDC), alcohol dehydrogenase (ADH), or any combination thereof. In some cases, one or more of the polynucleotides can be native to the microorganism. In some cases, one or more of the polynucleotides can be heterologous to the microorganism.

In some cases, L-threonine ammonia lyase can catalyze the conversion of L-threonine to 2-oxybutanoate and ammonia. In some cases, 2-ethylmalate synthase can catalyze the conversion of 2-oxybutanoate and acetyl-CoA to 2-ethylmalate. In some cases, isopropylmalate isomerase can catalyze the conversion of 2-ethylmalate to 3-ethylmalate. In some cases, 3-isopropylmalate dehydrogenase can catalyze the conversion of 3-ethylmalate to 2-ketovalerate, CO₂ and NADH. In some cases, KDC can catalyze the conversion of 2-ketovalerate to butryaldehyde. In some cases, ADH can catalyze the conversion of butyraldehyde to 1-butanol. In some cases, the L-threonine ammonia lyase can comprise a nucleotide sequence that is substantially similar to SEQ ID NO: 60. In some cases, the L-threonine ammonia lyase can comprise an amino acid sequence that is substantially similar to SEQ ID NO: 59. In some cases, the 2-ethylmalate synthase can comprise a nucleotide sequence that is substantially similar to SEQ ID NO:62. In some cases, the 2-ethylmalate synthase can comprise an amino acid sequence that is substantially similar to SEQ ID NO: 61. In some cases, the isopropylmalate isomerase can comprise a nucleotide sequence that is substantially similar to SEQ ID NO: 64 or 66. In some cases, the isopropylmalate isomerase can comprise an amino acid sequence that is substantially similar to SEQ ID NO: 63 or 65. In some cases, the KDC can comprise a nucleotide sequence that is substantially similar to any one of SEQ ID NOs: 36, 38, 40, 42, 44, 46 or 48. In some cases, the KDC can comprise an amino acid sequence that is substantially similar to any one of SEQ ID NOs: 35, 37 39, 41, 43, 45, or 47. In some cases, the ADH can be encoded by a polynucleotide that is substantially similar to any one of SEQ ID NOs: 50, 52, 54, 56 or 58. In some cases, the ADH can be encoded by an amino acid that is substantially similar to any one of SEQ ID NOs: 49, 51, 53, 55 or 57.

In some cases, the codons can be optimized based on the microorganism in which the genes will be provided or the enzymes will be expressed. In some cases, one or more the nucleotide sequences encoding for one or more of the enzymes in the 1-butanol pathway can be driven by a molecular switch. In some cases, the molecular switch can be turned on or off by a chemical substance such as the ones described throughout. In some cases, the chemical substance can be a rare earth metal. In some cases, the rare earth metal can be lanthanum.

### Ethanol

In some cases, polynucleotides encoding for enzymes of the ethanol pathway can be used. For example, the microorganism can contain (either endogenously or heterologous) one or more polynucleotides encoding for an enzyme from the Enzyme Class EC 1.2.4.1 and/or EC 1.1.1.1. In some cases, the polynucleotides encode for one or more polypeptides that is pyruvate decarboxylase (PDC) and/or alcohol dehydrogenase (ADH). In some cases, one or more of the polynucleotides can be native to the microorganism. In some cases, one or more of the polynucleotides can be heterologous to the microorganism.

In some cases, pyruvate decarboxylase can catalyze the conversion of pyruvate to acetaldehyde. In some cases, the alcohol dehydrogenase can catalyze the conversion of acetaldehyde into ethanol.

In some cases, the pyruvate decarboxylase can comprise a nucleotide sequence that is substantially similar to SEQ ID NO: 68. In some cases, the pyruvate decarboxylase can comprise an amino acid sequence that is substantially similar to SEQ ID NO: 67. In some cases, the alcohol dehydrogenase can comprise a nucleotide sequence that is substantially similar to SEQ ID NO: 70. In some cases, the alcohol dehydrogenase can comprise an amino acid sequence that is substantially similar to SEQ ID NO: 69.

In some cases, the codons can be optimized based on the microorganism in which the genes will be provided or the enzymes will be expressed. In some cases, one or more the nucleotide sequences encoding for one or more of the enzymes in the ethanol pathway can be driven by a molecular switch. In some cases, the molecular switch can be turned on or off by a chemical substance such as described throughout. In some cases, the chemical substance can be a rare earth metal. In some cases, the rare earth metal can be lanthanum.

### Fatty (or Aliphatic Long Chain) Alcohols

In some cases, polynucleotides encoding for enzymes of the fatty alcohol pathway can be used. For example, the microorganism can contain (either endogenously or heterologous) one or more polynucleotides encoding for an enzyme from the Enzyme Class EC 1.2.1.50. In some cases, the polynucleotides encode for one or more polypeptides that is a fatty-acyl-CoA reductase (FAR). In some cases, one or more of the polynucleotides can be native to the microorganism. In some cases, one or more of the polynucleotides can be heterologous to the microorganism.

In some cases, the fatty-acyl-CoA reductase can catalyze the conversion of fatty acetyl-CoA to a fatty alcohol.

In some cases, the fatty-acyl-CoA reductase can comprise a nucleotide sequence that is substantially similar to SEQ ID NO: 72. In some cases, the fatty-acyl-CoA reductase can comprise an amino acid sequence that is substantially similar to SEQ ID NO: 71.

In some cases, the codons can be optimized based on the microorganism in which the genes will be provided or the enzymes will be expressed. In some cases, one or more the nucleotide sequences encoding for one or more of the enzymes in the fatty alcohol pathway can be driven by a molecular switch. In some cases, the molecular switch can be turned on or off by a chemical substance such as described throughout. In some cases, the chemical substance can be a rare earth metal. In some cases, the rare earth metal can be lanthanum.

### Fatty Acid Methyl Ester

In some cases, polynucleotides encoding for enzymes of the fatty acid methyl ester pathway can be used. For example, the microorganism can contain (either endogenously or heterologous) one or more polynucleotides encoding for an enzyme from the Enzyme Class EC 2.3.1.75. In some cases, the polynucleotides encode for one or more polypeptides that is a WES polypeptide. In some cases, one or more of the polynucleotides can be native to the microorganism. In some cases, one or more of the polynucleotides can be heterologous to the microorganism.

In some cases, the WES polypeptide can catalyze the conversion of fatty acyl-CoA and alcohols to fatty acid esters.

In some cases, the WES polypeptide can comprise a nucleotide sequence that is substantially similar to any one of SEQ ID NOs: 74, 76, 78, 80, or 82. In some cases, the WES polypeptide can comprise an amino acid sequence that is substantially similar to any one of SEQ ID NOs: 73, 75, 77, 79, or 81.

In some cases, the codons can be optimized based on the microorganism in which the genes will be provided or the enzymes will be expressed. In some cases, one or more the nucleotide sequences encoding for one or more of the enzymes in the fatty acid methyl ester pathway can be driven by a molecular switch. In some cases, the molecular switch can be turned on or off by a chemical substance such as described throughout. In some cases, the chemical substance can be a rare earth metal. In some cases, the rare earth metal can be lanthanum.

### Vectors

Since some of the enzymes described throughout are not native to some microorganisms, expression vectors can be used to express the desired enzymes within most microorganism and cells. For example, methylotrophs such as methanotrophs, do not naturally express some enzymes of the 2,3-BDO; 1,4-BDO; isobutyraldehyde; isobutanol; 1-butanol; n-butanol; ethanol; fatty alcohols; and fatty acid methyl ester pathways. Therefore, in some cases, the heterologous enzymes can be expressed using certain expression vectors. Vector constructs prepared for introduction into the host microorganisms described throughout can typically, but not always, comprise a replication system (i.e. vector) recognized by the host. In some cases, the vector includes the intended polynucleotide fragment encoding the desired polypeptide and, optionally, transcription and translational initiation regulatory sequences operably linked to the polypeptide-encoding segment. Expression vectors can include, for example, an origin of replication or autonomously replicating sequence (ARS), expression control sequences, a promoter, an enhancer and necessary processing information sites, such as ribosome-binding sites, RNA splice sites, polyadenylation sites, transcriptional terminator sequences, mRNA stabilizing sequences, polynucleotides homologous to host chromosomal DNA, and/or a multiple cloning site. Signal peptides may also be included where appropriate, for example from secreted polypeptides of the same or related species, which allow the protein to cross and/or lodge in cell membranes or be secreted from the cell.

In some cases, the promoter used in the vector can be sensitive to a chemical substance such as described throughout. For example, in the presence of the chemical substance, the promoter is either activated or deactivated. In some cases, the chemical substance can be a rare earth metal. In some cases, the rare earth metal can be lanthanum or cerium. In some cases, the rare earth metal can be praseodymium or neodymium.

The vectors can be constructed using standard methods (sec, e.g., Sambrook et al., Molecular Biology: A Laboratory Manual, Cold Spring Harbor, N.Y. 1989; and Ausubel, et al., Current Protocols in Molecular Biology, Greene Publishing, Co. N.Y., 1995).

Manipulation of polynucleotides that encode the enzymes disclosed throughout is typically carried out in recombinant vectors. Vectors which may be employed include bacterial plasmids, bacteriophage, artificial chromosomes, episomal vectors and gene expression vectors. Vectors may be selected to accommodate a polynucleotide encoding a protein of a desired size. Following production of a selected vector, a suitable host cell (e.g., the microorganisms described herein) is transfected or transformed with the vector. Each vector contains various functional components, which generally include a cloning site, an origin of replication and at least one selectable marker gene. A vector may additionally possess one or more of the following elements: an enhancer, promoter, a transcription termination sequence and/or other signal sequences. Such sequence elements may be optimized for the selected host species. Such sequence elements may be positioned in the vicinity of the cloning site, such that they are operatively linked to the gene encoding a preselected enzyme.

Vectors, including cloning and expression vectors, may contain polynucleotides that enable the vector to replicate in one or more selected microorganisms. For example, the sequence may be one that enables the vector to replicate independently of the host chromosomal DNA and may include origins of replication or autonomously replicating sequences. Such sequences are well known for a variety of bacteria, yeast and viruses. For example, the origin of replication from the plasmid pBR322 is suitable for most gram-negative bacteria, the origin of replication for 2 micron plasmid is suitable for yeast, and various viral origins of replication (e.g. SV40, adenovirus) are useful for cloning vectors.

A cloning or expression vector may contain a selection gene, also referred to as a selectable marker. This gene encodes a protein necessary for the survival or growth of transformed microorganisms in a selective culture medium. Microorganisms not transformed with the vector containing the selection gene will therefore not survive in the culture medium. Typical selection genes encode proteins that confer resistance to antibiotics and other toxins, e.g. ampicillin, neomycin, methotrexate, hygromycin, thiostrepton, apramycin or tetracycline, complement auxotrophic deficiencies, or supply critical nutrients not available in the growth media.

The replication of vectors may be performed in E. coli. An example of a E. coli-selectable marker is the β-lactamase gene, which confers resistance to the antibiotic ampicillin. These selectable markers can be obtained from E. coli plasmids, such as pBR322 or a pUC plasmid such as pUC18 or pUC19, or pUC119.

### Promoters

Vectors may contain a promoter that is recognized by the host microorganism. The promoter may be operably linked to a coding sequence of interest. Such a promoter may be inducible, repressible, or constitutive. Polynucleotides are operably linked when the polynucleotides are in a relationship permitting them to function in their intended manner.

Different promoters can be used to drive the expression of the genes. For example, if temporary gene expression (i.e., non-constitutively expressed) is desired, expression can be driven by inducible or repressible promoters. The molecular switch can in some cases comprise these inducible or repressible promoters.

In some cases, the desired gene is expressed temporarily. In other words, the desired gene is not constitutively expressed. The expression of the desired gene can be driven by inducible or repressible promoters, which functions as a molecular switch. Examples of inducible or repressible switches include, but are not limited to, those promoters inducible or repressible by: (a) sugars such as arabinose and lactose (or non metabolizable analogs, e.g., isopropyl β-D-1-thiogalactopyranoside (IPTG)); (b) metals such as copper or calcium (or rare earth metals such as lanthanum or cerium); (c) temperature; (d) Nitrogen-source; (e) oxygen; (f) cell state (growth or stationary); (g) metabolites such as phosphate; (h) CRISPRi; (i) jun; (j) fos, (k) metallothionein and/or (l) heat shock. These switches can be used in a methanotroph system. An example of an inducible switch that can be used within methanotrophs is a pBAD, pXoxF or pTRC promoter. An example of a repressible switch that can be used within methanotrophs is a pMxaF promoter.

Inducible or repressible switches that can be particularly useful are switches that are responsive to sugars and rare earth metals. For example, promoters that are sensitive to the sugar arabinose can be used as an inducible switch. In some cases, arabinose switches can be used to drive expression of one or more genes. For example, in the presence arabinose, a desired vector or expression of a gene set can be “turned-on.” The arabinose switch can turn on the expression of a desired gene.

Other particularly useful switches can be rare earth metal switches, such as lanthanum sensitive switches (also simply known as a lanthanum switch). In some cases, the lanthanum switch can be a repressible switch that can be used to repress expression of one or more genes, until the repressor is removed (e.g., in this case lanthanum), after which the genes are “turned-on”. For example, in the presence the rare earth metal lanthanum, the desired gene set or vector can be “turned-off.” The lanthanum switch can be turned off (and expression of the genes induced) by either removing the lanthanum from the media or diluting the lanthanum in the media to levels where its repressible effects are reduced, minimized, or eliminated. Other rare earth metal switches can be used, such as those disclosed throughout.

Constitutively expressed promoters can also be used in the vector systems herein. For example, the expression of one or more desired genes can be controlled by constitutively active promoters. Examples of such promoters include but are not limited to pXoxF, pMxaF and p.Bba.J23111.

Promoters suitable for use with prokaryotic hosts may include, for example, the a-lactamase and lactose promoter systems, alkaline phosphatase, the tryptophan (trp) promoter system, the erythromycin promoter, apramycin promoter, hygromycin promoter, methylenomycin promoter and hybrid promoters such as the tac promoter. Promoters for use in bacterial systems will also generally contain a Shine-Dalgarno sequence operably linked to the coding sequence.

Generally, a strong promoter may be employed to provide for high level transcription and expression of the desired product. For example, promoters that can be used include but are not limited to a pMxaF promoter. In some cases, a mutation can increase the strength of the promoter and therefore result in elevated levels of expression.

In some cases however, a weaker promoter is desired. For example, this is the case where too much expression of a certain gene results in a detrimental effect (e.g., the killing of cells). A weak promoter can be used, for example a pBAD promoter. However, in some cases, a weaker promoter can be made by mutation. For example, the pmxaF promoters can be mutated to be weaker.

One or more promoters of a transcription unit can be an inducible promoter. For example, a GFP can be expressed from a constitutive promoter while an inducible promoter is used to drive transcription of a gene coding for one or more enzymes as disclosed herein and/or the amplifiable selectable marker.

Some vectors may contain prokaryotic sequences that facilitate the propagation of the vector in bacteria. Thus, the vectors may have other components such as an origin of replication (e.g., a polynucleotide that enables the vector to replicate in one or more selected microorganisms), antibiotic resistance genes for selection in bacteria, and/or an amber stop codon which can permit translation to read through the codon. Additional selectable gene(s) may also be incorporated. Generally, in cloning vectors, the origin of replication is one that enables the vector to replicate independently of the host chromosomal DNA, and includes origins of replication or autonomously replicating sequences. Such sequences can include the ColEl origin of replication in bacteria or other known sequences.

### Genes

The genes described throughout can all have a promoter driving their expression. The methods described herein, e.g., genome editing and expression inhibition using Cas, can be used to edit the polynucleotide of the promoters or used to inhibit the effectiveness of the promoters. Inhibition can be done by blocking the transcription machinery (e.g., transcription factors) from binding to the promoter or by altering the promoter in such a way that the transcription machinery no longer recognizing the promoter sequence.

The vectors described throughout can also comprise a polynucleotide encoding for one or more of the genes within the 2,3-BDO; 1,4-BDO; isobutyraldehyde; isobutanol; 1-butanol; n-butanol; ethanol; fatty alcohols; and fatty acid methyl ester pathway. These vectors can also contain one or more regulatory elements (inducible and/or repressible promoters) that control the expression of the genes within the vectors. In some cases, the vectors can include switches, including but not limited to inducible or repressible switches, e.g., an arabinose or lanthanum switches. These genes can be heterologous to the microorganism in which the vector is contacted with (and eventually transformed with).

The genes used in the vectors can be any genes described throughout the application. For example, the genes of the 2,3-BDO; 1,4-BDO; isobutyraldehyde; isobutanol; 1-butanol; n-butanol; ethanol; fatty alcohols; and fatty acid methyl ester pathways. These enzymes can be encoded by a polynucleotide or by an polypeptide that is at least 60%, 65%, 70%, 75%, 80%, 85%, 90%, 91%, 92%, 93%, 94%, 95%, 96%, 97%, 98%, 99%, 99.9%, 99.99%, 99.999%, or 99.9999% identical to any one of SEQ ID NOs: 1 to 82.

The genes that are inserted into a microorganism can be heterologous to the microorganism itself. For example, if the microorganism is a methanotroph, the inserted genes can, for example, be from yeast, a bacterium, or a different species of methanotroph. Further, the genes can be endogenously part of the genome of the microorganism.

### III. Method of Making the Genetically Modified Microorganisms

The genetically modified microorganisms disclosed throughout can be made by a variety of ways. A microorganism may be modified (e.g., genetically engineered) by any method to comprise and/or express one or more polynucleotides encoding for enzymes in a pathway that catalyze a conversion of a fermentable carbon source (e.g., a C1 carbon) to one or more intermediates in a pathway for the production of desired valuable chemicals, such as 2,3-BDO; 1,4-BDO; isobutyraldehyde; isobutanol; 1-butanol (a.k.a. n-butanol); ethanol; fatty (or aliphatic long chain) alcohols; and fatty acid methyl esters. Such enzymes may include those discussed throughout. For example, one or more of any of the genes discussed throughout can be inserted into a microorganism. The genes can be inserted by an expression vector. The genes can also be under the control of one or more different/same promoters or the one or more genes can be under the control of a switch, such as an inducible or repressible promoter, e.g., an arabinose switch, isopropyl β-D-1-thiogalactopyranoside (IPTG) switch, or a rare earth metal switch. The genes can also be stably integrated into the genome of the microorganism. In some cases, the genes can be expressed in an episomally vector.

The microorganism used in this method can be any described above, including but not limited to a prokaryote. Other microorganisms such as bacteria, yeast, or algae can be used. Microorganisms of particular interest include methanotrophs, such as those from the genera Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, or Methyloacidophilum. One desired species can include a Methylococcus capsulatus.

An exemplary method of making a genetically modified microorganism disclosed herein is contacting (or transforming) a microorganism with a nucleic acid that expresses at least one heterologous gene encoding i) an acetoin reductase; ii) an alpha-acetolactate decarboxylase (budA); iii) a acetolactate synthase, or iv) any combination thereof. The microorganism can be any microorganism that is capable of converting a C1 carbon to a product. In some cases, the product is 2,3-BDO. For example, another exemplary method of making a genetically modified microorganism disclosed herein is contacting (or transforming) a microorganism with a nucleic acid that expresses at least one heterologous gene encoding i) succinyl-CoA synthetase (sucC), ii) CoA-dependent succinate semialdehyde dehydrogenase (sucD), iii) 4-hydroxybutyrate dehydrogenase (4hbD), iv) 4-hydroxybutyryl-CoA transferase (cat2), v) aldehyde dehydrogenase (ald), vi) alcohol dehydrogenase (adh), or vii) any combination thereof. The microorganism can be any microorganism that is capable of converting a C1 carbon to a product. In some cases, the product is 1,4-BDO.

Another exemplary method of making a genetically modified microorganism disclosed herein is contacting (or transforming) a microorganism with a nucleic acid that expresses at least one heterologous gene encoding i) acetolactate synthase (AlsS); ii) ketol-acid reductoisomerase (KARI); iii) dihydroxy-acid dehydratase (DHAD); iv) 2-keto acid decarboxylase (KDC), or v) any combination thereof. The microorganism can be any microorganism that is capable of converting a C1 carbon to a product. In some cases, the product is isobutyraldehyde.

Another exemplary method of making a genetically modified microorganism disclosed herein is contacting (or transforming) a microorganism with a nucleic acid that expresses at least one heterologous gene encoding i) acetolactate synthase (AlsS); ii) ketol-acid reductoisomerase (KARI); iii) dihydroxy-acid dehydratase (DHAD); iv) 2-keto acid decarboxylase (KDC), v) alcohol dehydrogenase (ADH) or vi) any combination thereof. The microorganism can be any microorganism that is capable of converting a C1 carbon to a product. In some cases, the product is isobutanol.

Another exemplary method of making a genetically modified microorganism disclosed herein is contacting (or transforming) a microorganism with a nucleic acid that expresses at least one heterologous gene encoding i) L-threonine ammonia lyase, ii) 2-ethylmalate synthase, isopropylmalate isomerase, iii) 2-ketoacid decarboxylase, iv) alcohol dehydrogenase, or v) any combination thereof. The microorganism can be any microorganism that is capable of converting a C1 carbon to a product. In some cases, the product is 1-butanol.

Another exemplary method of making a genetically modified microorganism disclosed herein is contacting (or transforming) a microorganism with a nucleic acid that expresses at least one heterologous gene encoding i) pyruvate decarboxylase (PDC), ii) alcohol dehydrogenase (ADH), or iii) any combination thereof. The microorganism can be any microorganism that is capable of converting a C1 carbon to a product. In some cases, the product is ethanol.

Another exemplary method of making a genetically modified microorganism disclosed herein is contacting (or transforming) a microorganism with a nucleic acid that expresses at least one heterologous gene encoding fatty-acyl-CoA reductase (FAR). The microorganism can be any microorganism that is capable of converting a C1 carbon to a product. In some cases, the product is a fatty (or aliphatic long chain) alcohol.

Another exemplary method of making a genetically modified microorganism disclosed herein is contacting (or transforming) a microorganism with a nucleic acid that expresses at least one heterologous gene encoding a WES polypeptide. The microorganism can be any microorganism that is capable of converting a C1 carbon to a product. In some cases, the product is a fatty acid methyl ester.

The genes that are inserted into a microorganism can be heterologous to the microorganism itself. For example, if the microorganism is a methanotroph, the inserted genes can, for example, be from yeast, a bacterium, or a different species of methanotroph. Further, the genes can be endogenously part of the genome of the microorganism.

The genes can be inserted into a microorganism through the use of vectors. In some cases, the genes can be inserted into the genome of the microorganism. In some cases, the genes can be episomally expressed within the genome of the microorganism. In some cases, both techniques can be used when two or more genes are inserted into a microorganism. For example, a gene can be inserted into the genome of a microorganism by, for example, use of an integration vector. Subsequently, an additional gene can be transformed into the microorganism through an episomal vector.

### Techniques for Genetic Modification

The microorganisms disclosed herein may be genetically engineered by using classic microbiological techniques. Some of such techniques are generally disclosed, for example, in Sambrook et al., 1989, Molecular Cloning: A Laboratory Manual, Cold Spring Harbor Labs Press.

The genetically modified microorganisms disclosed herein may include a polynucleotide that has been inserted, deleted or modified (i.e., mutated; e.g., by insertion, deletion, substitution, and/or inversion of nucleotides), in such a manner that such modifications provide the desired effect of expression (e.g., over-expression) of one or more enzymes as provided herein within the microorganism. Genetic modifications which result in an increase in gene expression or function can be referred to as amplification, overproduction, overexpression, activation, enhancement, addition, or up-regulation of a gene. Addition of a gene to increase gene expression can include maintaining the gene(s) on replicating plasmids or integrating the cloned gene(s) into the genome of the production microorganism. Furthermore, increasing the expression of desired genes can include operatively linking the cloned gene(s) to native or heterologous transcriptional control elements.

Where desired, the expression of one or more of the enzymes provided herein is under the control of a regulatory sequence that controls directly or indirectly the enzyme expression in a time-dependent fashion during the fermentation. Inducible promoters can be used to achieve this.

In some cases, a microorganism is transformed or transfected with a genetic vehicle, such as an expression vector comprising a heterologous polynucleotide sequence coding for the enzymes are provided herein. In some cases, the vector(s) can be an episomal vector, or the gene sequence can be integrated into the genome of the microorganism, or any combination thereof. In some cases, the vectors comprising the heterologous polynucleotide sequence encoding for the enzymes provided herein are integrated into the genome of the microorganism.

To facilitate insertion and expression of different genes coding for the enzymes as disclosed herein from the constructs and expression vectors, the constructs may be designed with at least one cloning site for insertion of any gene coding for any enzyme disclosed herein. The cloning site may be a multiple cloning site, e.g., containing multiple restriction sites.

### Transfection

Standard transfection techniques can be used to insert genes into a microorganism. As used herein, the term “transfection” or “transformation” can refer to the insertion of an exogenous nucleic acid or polynucleotide into a host cell. The exogenous nucleic acid or polynucleotide may be maintained as a non-integrated vector, for example, a plasmid or episomal vector, or alternatively, may be integrated into the host cell genome. The term transfecting or transfection is intended to encompass all conventional techniques for introducing nucleic acid or polynucleotide into microorganisms. Examples of transfection techniques include, but are not limited to, calcium phosphate precipitation, DEAE-dextran-mediated transfection, lipofection, electroporation, microinjection, rubidium chloride or polycation mediated transfection, protoplast fusion, and sonication. The transfection method that provides optimal transfection frequency and expression of the construct in the particular host cell line and type is favored. For stable transfectants, the constructs are integrated so as to be stably maintained within the host chromosome. In some cases, the preferred transfection is a stable transfection. In some cases, the integration of the gene occurs at a specific locus within the genome of the microorganism.

### Transformation

Expression vectors or other nucleic acids may be introduced to selected microorganisms by any of a number of suitable methods. For example, vector constructs may be introduced to appropriate cells by any of a number of transformation methods for plasmid vectors. Standard calcium-chloride-mediated bacterial transformation is still commonly used to introduce naked DNA to bacteria (see, e.g., Sambrook et al., 1989, Molecular Cloning, A Laboratory Manual, Cold Spring Harbor Laboratory Press, Cold Spring Harbor, N.Y.), but electroporation and conjugation may also be used (see, e.g., Ausubel et al., 1988, Current Protocols in Molecular Biology, John Wiley & Sons, Inc., NY, N.Y.).

For the introduction of vector constructs to yeast or other fungal cells, chemical transformation methods may be used (e.g., Rose et al., 1990, Methods in Yeast Genetics, Cold Spring Harbor Laboratory Press, Cold Spring Harbor, N.Y.). Transformed cells may be isolated on selective media appropriate to the selectable marker used. Alternatively, or in addition, plates or filters lifted from plates may be scanned for GFP fluorescence to identify transformed clones.

For the introduction of vectors comprising differentially expressed sequences to certain types of cells, the method used may depend on the form of the vector. Plasmid vectors may be introduced by any of a number of transfection methods, including, for example, lipid-mediated transfection (“lipofection”), DEAE-dextran-mediated transfection, electroporation or calcium phosphate precipitation (see, e.g., Ausubel et al., 1988, Current Protocols in Molecular Biology, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. Many companies offer kits and ways for this type of transfection.

The host cell may be capable of expressing the construct encoding the desired protein, processing the protein and transporting a secreted protein to the cell surface for secretion. Processing includes co- and post-translational modification such as leader peptide cleavage, GPI attachment, glycosylation, ubiquitination, and disulfide bond formation.

Microorganisms can be transformed or transfected with the above-described expression vectors or polynucleotides coding for one or more enzymes as disclosed herein and cultured in nutrient media modified as appropriate for the specific microorganism, inducing promoters, selecting transformants, or amplifying the genes encoding the desired sequences. In some cases, electroporation methods can be used to deliver an expression vector.

Expression of a vector (and the gene contained in the vector) can be verified by an expression assay, for example, qPCR or by measuring levels of RNA. Expression level can be indicative also of copy number. For example, if expression levels are extremely high, this can indicate that more than one copy of a gene was integrated in a genome. Alternatively, high expression can indicate that a gene was integrated in a highly transcribed area, for example, near a highly expressed promoter. Expression can also be verified by measuring protein levels, such as through Western blotting.

### CRISPR/Cas System

The methods disclosed throughout can involve pinpoint insertion of genes or the deletion of genes (or parts of genes). Methods described herein can use a CRISPR/cas system. For example, double-strand breaks (DSBs) can be generated using a CRISPR/cas system, e.g., a type II CRISPR/cas system. A Cas enzyme used in the methods disclosed herein can be Cas9, which catalyzes DNA cleavage. Enzymatic action by Cas9 from Streptococcus pyogenes or any closely related Cas9 can generate double stranded breaks at target site sequences which hybridize to 20 nucleotides of a guide sequence and have a protospacer-adjacent motif (PAM) following the 20 nucleotides of the target sequence.

A vector can be operably linked to an enzyme-coding sequence encoding a CRISPR enzyme, such as a Cas protein. Cas proteins that can be used include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof. An unmodified CRISPR enzyme can have DNA cleavage activity, such as Cas9. A CRISPR enzyme can direct cleavage of one or both strands at a target sequence, such as within a target sequence and/or within a complement of a target sequence. For example, a CRISPR enzyme can direct cleavage of one or both strands within 1, 2, 3, 4, 5, 6, 7, 8, 9, 10, 15, 20, 25, 30, 35, 40, 45, 50, 60, 70, 80, 90, 100, 125, 150, 175, 200, 300, 400, 500, or more base pairs from the first or last nucleotide of a target sequence. A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used.

A vector that encodes a CRISPR enzyme comprising one or more nuclear localization sequences (NLSs) can be used. For example, there can be 1, 2, 3, 4, 5, 6, 7, 8, 9, 10 NLSs used. A CRISPR enzyme can comprise the NLSs at or near the ammo-terminus (e.g., 1, 2, 3, 4, 5, 6, 7, 8, 9, 10 NLSs), or at or near the carboxy-terminus (e.g., 1, 2, 3, 4, 5, 6, 7, 8, 9, 10 NLSs), or any combination of these (e.g., one or more NLS at the ammo-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise at most 6 NLSs. An NLS is considered near the N- or C-terminus when the nearest amino acid to the NLS is within 50 amino acids along a polypeptide chain from the N- or C-terminus, e.g., within 1, 2, 3, 4, 5, 10, 15, 20, 25, 30, 40, or 50 amino acids.

### Guide RNA

As used herein, the term “guide RNA” and its grammatical equivalents can refer to an RNA which can be specific for a target DNA and can form a complex with Cas protein. An RNA/Cas complex can assist in “guiding” Cas protein to a target DNA.

A method disclosed herein also can comprise introducing into a cell or embryo at least one guide RNA or nucleic acid, e.g., DNA encoding at least one guide RNA. A guide RNA can interact with a RNA-guided endonuclease to direct the endonuclease to a specific target site, at which site the 5′ end of the guide RNA base pairs with a specific protospacer sequence in a chromosomal sequence.

A guide RNA can comprise two RNAs, e.g., CRISPR RNA (crRNA) and transactivating crRNA (tracrRNA). A guide RNA can sometimes comprise a single-chain RNA, or single guide RNA (sgRNA) formed by fusion of a portion (e.g., a functional portion) of crRNA and tracrRNA. A guide RNA can also be a dualRNA comprising a crRNA and a tracrRNA. Furthermore, a crRNA can hybridize with a target DNA.

As discussed above, a guide RNA can be an expression product. For example, a DNA that encodes a guide RNA can be a vector comprising a sequence coding for the guide RNA. A guide RNA can be transferred into a cell or microorganism by transfecting the cell or microorganism with an isolated guide RNA or plasmid DNA comprising a sequence coding for the guide RNA and a promoter. A guide RNA can also be transferred into a cell or microorganism in other way, such as using virus-mediated gene delivery.

A guide RNA can be isolated. For example, a guide RNA can be transfected in the form of an isolated RNA into a cell or microorganism. A guide RNA can be prepared by in vitro transcription using any in vitro transcription system. A guide RNA can be transferred to a cell in the form of isolated RNA rather than in the form of plasmid comprising encoding sequence for a guide RNA.

A guide RNA can comprise three regions: a first region at the 5′ end that can be complementary to a target site in a chromosomal sequence, a second internal region that can form a stem loop structure, and a third 3′ region that can be single-stranded. A first region of each guide RNA can also be different such that each guide RNA guides a fusion protein to a specific target site. Further, second and third regions of each guide RNA can be identical in all guide RNAs.

A first region of a guide RNA can be complementary to sequence at a target site in a chromosomal sequence such that the first region of the guide RNA can base pair with the target site. In some cases, a first region of a guide RNA can comprise from 10 nucleotides to 25 nucleotides (i.e., from 10 nts to 25 nts; or 10 nts to 25 nts; or from 10 nts to 25 nts; or from 10 nts to 25 nts) or more. For example, a region of base pairing between a first region of a guide RNA and a target site in a chromosomal sequence can be 10, 11, 12, 13, 14, 15, 16, 17, 18, 19, 20, 22, 23, 24, 25, or more nucleotides in length. Sometimes, a first region of a guide RNA can be 19, 20, or 21 nucleotides in length.

A guide RNA can also comprises a second region that forms a secondary structure. For example, a secondary structure formed by a guide RNA can comprise a stem (or hairpin) and a loop. A length of a loop and a stem can vary. For example, a loop can range from 3 to 10 nucleotides in length, and a stem can range from 6 to 20 base pairs in length. A stem can comprise one or more bulges of 1 to 10 nucleotides. The overall length of a second region can range from 16 to 60 nucleotides in length. For example, a loop can be 4 nucleotides in length and a stem can be 12 base pairs.

A guide RNA can also comprise a third region at the 3′ end that can be essentially single-stranded. For example, a third region is sometimes not complementarity to any chromosomal sequence in a cell of interest and is sometimes not complementarity to the rest of a guide RNA. Further, the length of a third region can vary. A third region can be more than 4 nucleotides in length. For example, the length of a third region can range from 5 to 60 nucleotides in length.

A guide RNA can be introduced into a cell or embryo as an RNA molecule. For example, a RNA molecule can be transcribed in vitro and/or can be chemically synthesized. An RNA can be transcribed from a synthetic DNA molecule, e.g., a gBlocks® gene fragment. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell or embryo of interest. A RNA coding sequence can be operably linked to a promoter sequence that is recognized by RNA polymerase III (Pol III). Plasmid vectors that can be used to express guide RNA include, but are not limited to, px330 vectors and px333 vectors. In some cases, a plasmid vector (e.g., px333 vector) can comprise two guide RNA-encoding DNA sequences.

A DNA sequence encoding a guide RNA can also be part of a vector. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. A DNA molecule encoding a guide RNA can also be linear. A DNA molecule encoding a guide RNA can also be circular.

When DNA sequences encoding an RNA-guided endonuclease and a guide RNA are introduced into a cell, each DNA sequence can be part of a separate molecule (e.g., one vector containing an RNA-guided endonuclease coding sequence and a second vector containing a guide RNA coding sequence) or both can be part of a same molecule (e.g., one vector containing coding (and regulatory) sequence for both an RNA-guided endonuclease and a guide RNA).

### Site-Specific Insertion

Insertion of the genes can be site-specific. For example, one or more genes can be inserted adjacent to a promoter.

Modification of a targeted locus of a microorganism can be produced by introducing DNA into microorganisms, where the DNA has homology to the target locus. DNA can include a marker gene, allowing for selection of cells comprising the integrated construct. Homologous DNA in a target vector can recombine with DNA at a target locus. A marker gene can be flanked on both sides by homologous DNA sequences, a 3′ recombination arm, and a 5′ recombination arm.

A variety of enzymes can catalyze insertion of foreign DNA into a microorganism genome. For example, site-specific recombinases can be clustered into two protein families with distinct biochemical properties, namely tyrosine recombinases (in which DNA is covalently attached to a tyrosine residue) and serine recombinases (where covalent attachment occurs at a serine residue). In some cases, recombinases can comprise Cre, ΦC31 integrase (a serine recombinase derived from Streptomyces phage ΦC31), or bacteriophage derived site-specific recombinases (including Flp, lambda integrase, bacteriophage HK022 recombinase, bacteriophage R4 integrase and phage TP901-1 integrase).

The CRISPR/Cas system can be used to perform site specific insertion. For example, a nick on an insertion site in the genome can be made by CRISPR/cas to facilitate the insertion of a transgene at the insertion site.

The methods described herein, can utilize techniques which can be used to allow a DNA or RNA construct entry into a host cell include, but are not limited to, calcium phosphate/DNA coprecipitation, microinjection of DNA into a nucleus, electroporation, bacterial protoplast fusion with intact cells, transfection, lipofection, infection, particle bombardment, sperm mediated gene transfer, or any other technique.

Certain aspects disclosed herein can utilize vectors (including the ones described above). Any plasmids and vectors can be used as long as they are replicable and viable in a selected host microorganism. Vectors known in the art and those commercially available (and variants or derivatives thereof) can be engineered to include one or more recombination sites for use in the methods. Vectors that can be used include, but not limited to eukaryotic expression vectors such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), pXT1, pSG5, pPbac, pMbac, pMClneo, and pOG44 (Stratagene, Inc.), and pYES2, pAC360, pBlueBa-cHis A, B, and C, pVL1392, pBlueBac111, pCDM8, pcDNA1, pZeoSV, pcDNA3, pREP4, pCEP4, and pEBVHis (Invitrogen, Corp.), and variants or derivatives thereof.

These vectors can be used to express a gene or portion of a gene of interest. A gene of portion or a gene can be inserted by using known methods, such as restriction enzyme-based techniques.

### IV. Other Methods

### Methods of Making Valued Chemicals

The genetically modified microorganisms described herein can be used to make valued chemicals, including but not limited to 2,3-BDO; 1,4-BDO; isobutyraldehyde; isobutanol; 1-butanol; n-butanol; ethanol; fatty (or aliphatic long chain) alcohols; and fatty acid methyl esters.

The microorganism can be any of the microorganisms discussed throughout including but not limited to a prokaryote, such as a methanotroph.

The carbon substrate can be any carbon substrate discussed throughout including but not limited to a C1 carbon substrate, such as methane.

The fermentation conditions used during the making of the valued chemicals can be any condition described throughout, such as in the presence or absence of a sugar or rare earth metal and a molecular switch. For example, the media can contain a sugar or rare earth metal when expression of a gene or genes is controlled by a molecular switch.

### 2,3-BDO

Disclosed herein is a method of making 2,3-BDO comprising contacting a genetically modified microorganism with a carbon substrate, where the microorganism comprises at least one heterologous gene under the control of a molecular switch encoding: i) an acetoin reductase; ii) an alpha-acetolactate decarboxylase (budA); iii) an AlsS; or iv) any combination thereof. In some cases, the at least one heterologous gene is integrated into the genome of the microorganism. The method can further comprise growing the microorganism to produce 2,3-BDO. At least one heterologous gene can be under the control of a switch, such as an inducible or repressible promoter that is responsive to the presence or absence of a component in the media, e.g., a sugar such as arabinose or a rare earth element such as lanthanum. Also the microorganism can be first grown in media that contains a rare earth metal (e.g., at least 1 μM lanthanum) and then subsequently the rare earth metal can be removed (e.g., by being diluted out of the media). This removal can occur before growing the microorganism to produce 2,3-BDO.

The 2,3-BDO that is produced from these methods can be substantially pure. The 2,3-BDO produced can be recovered. Additionally, non-2,3-BDO products (i.e., by-products) can also be recovered, such as 2-acetolactate and acetoin.

### 1,4-BDO

Disclosed herein is a method of making 1,4-BDO comprising contacting a genetically modified microorganism with a carbon substrate, where the microorganism comprises at least one heterologous gene under the control of a molecular switch encoding: i) succinyl-CoA synthetase (sucC), ii) CoA-dependent succinate semialdehyde dehydrogenase (sucD), iii) 4-hydroxybutyrate dehydrogenase (4hbD), iv) 4-hydroxybutyryl-CoA transferase (cat2), v) aldehyde dehydrogenase (ald), vi) alcohol dehydrogenase (adh), or vii) any combination thereof. In some cases, the at least one heterologous gene is integrated into the genome of the microorganism. The method can further comprise growing the microorganism to produce 1,4-BDO. At least one heterologous gene can be under the control of a switch, such as an inducible or repressible promoter that is responsive to the presence or absence of a component in the media, e.g., a sugar such as arabinose or a rare earth element such as lanthanum. Also the microorganism can be first grown in media that contains a rare earth metal (e.g., at least 1 μM lanthanum) and then subsequently the rare earth metal can be removed (e.g., by being diluted out of the media). This removal can occur before growing the microorganism to produce 1,4-BDO.

The 1,4-BDO that is produced from these methods can be substantially pure. The 1,4-BDO produced can be recovered. Additionally, non-1,4-BDO products (i.e., by-products) can also be recovered, such as succinyl coA, succinyl semialdehyde, γ-hydroxybutyrate, 4-hydroxybutyryl coA, and/or 4-hydroxybutyraldehyde.

### Isobutyraldehyde

Disclosed herein is a method of making isobutyraldehyde comprising contacting a genetically modified microorganism with a carbon substrate, where the microorganism comprises at least one heterologous gene under the control of a molecular switch encoding: i) acetolactate synthase (AlsS); ii) ketol-acid reductoisomerase (KARI); iii) dihydroxy-acid dehydratase (DHAD); iv) 2-keto acid decarboxylase (KDC), or v) any combination thereof. In some cases, the at least one heterologous gene is integrated into the genome of the microorganism. The method can further comprise growing the microorganism to produce isobutyraldehyde. At least one heterologous gene can be under the control of a switch, such as an inducible or repressible promoter that is responsive to the presence or absence of a component in the media, e.g., a sugar such as arabinose or a rare earth element such as lanthanum. Also the microorganism can be first grown in media that contains a rare earth metal (e.g., at least 1 μM lanthanum) and then subsequently the rare earth metal can be removed (e.g., by being diluted out of the media). This removal can occur before growing the microorganism to produce isobutyraldehyde.

The isobutyraldehyde that is produced from these methods can be substantially pure. The isobutyraldehyde produced can be recovered. Additionally, non-isobutyraldehyde products (i.e., by-products) can also be recovered, such as pyruvate, 2-acetolactate, 2,3-dihydroxyisovalerate, and ketoisovalerate.

### Isobutanol

Disclosed herein is a method of making isobutanol comprising contacting a genetically modified microorganism with a carbon substrate, where the microorganism comprises at least one heterologous gene under the control of a molecular switch encoding: i) acetolactate synthase (AlsS); ii) ketol-acid reductoisomerase (KARI); iii) dihydroxy-acid dehydratase (DHAD); iv) 2-keto acid decarboxylase (KDC), v) alcohol dehydrogenase (ADH) or vi) any combination thereof. In some cases, the at least one heterologous gene is integrated into the genome of the microorganism. The method can further comprise growing the microorganism to produce isobutanol. At least one heterologous gene can be under the control of a switch, such as an inducible or repressible promoter that is responsive to the presence or absence of a component in the media, e.g., a sugar such as arabinose or a rare earth element such as lanthanum. Also the microorganism can be first grown in media that contains a rare earth metal (e.g., at least 1 μM lanthanum) and then subsequently the rare earth metal can be removed (e.g., by being diluted out of the media). This removal can occur before growing the microorganism to produce isobutanol.

The isobutanol that is produced from these methods can be substantially pure. The isobutanol produced can be recovered. Additionally, non-isobutanol products (i.e., by-products) can also be recovered, such as pyruvate, 2-acetolactate, 2,3-dihydroxyisovalerate, and ketoisovalerate, isobutyraldehyde.

### 1-butanol (a.k.a. n-butanol)

Disclosed herein is a method of making 1-butanol comprising contacting a genetically modified microorganism with a carbon substrate, where the microorganism comprises at least one heterologous gene under the control of a molecular switch encoding: i) L-threonine ammonia lyase, ii) 2-ethylmalate synthase, isopropylmalate isomerase, iii) 2-ketoacid decarboxylase, iv) alcohol dehydrogenase, or v) any combination thereof. In some cases, the at least one heterologous gene is integrated into the genome of the microorganism. The method can further comprise growing the microorganism to produce 1-butanol. At least one heterologous gene can be under the control of a switch, such as an inducible or repressible promoter that is responsive to the presence or absence of a component in the media, e.g., a sugar such as arabinose or a rare earth element such as lanthanum. Also the microorganism can be first grown in media that contains a rare earth metal (e.g., at least 1 μM lanthanum) and then subsequently the rare earth metal can be removed (e.g., by being diluted out of the media). This removal can occur before growing the microorganism to produce 1-butanol.

The 1-butanol that is produced from these methods can be substantially pure. The 1-butanol produced can be recovered. Additionally, non-1-butanol products (i.e., by-products) can also be recovered, such as acetyl-CoA, acetoacetyl-CoA, 3-hydroxybutyryl-CoA, crotonyl-CoA, butyryl-CoA, and butylaldehyde.

### Ethanol

Disclosed herein is a method of making ethanol comprising contacting a genetically modified microorganism with a carbon substrate, where the microorganism comprises at least one heterologous gene under the control of a molecular switch encoding: i) pyruvate decarboxylase (PDC), ii) alcohol dehydrogenase (ADH), or iii) any combination thereof. In some cases, at least one heterologous gene is integrated into the genome of the microorganism. The method can further comprise growing the microorganism to produce ethanol. At least one heterologous gene can be under the control of a switch, such as an inducible or repressible promoter that is responsive to the presence or absence of a component in the media, e.g., a sugar such as arabinose or a rare earth element such as lanthanum. Also the microorganism can be first grown in media that contains a rare earth metal (e.g., at least 1 μM lanthanum) and then subsequently the rare earth metal can be removed (e.g., by being diluted out of the media). This removal can occur before growing the microorganism to produce ethanol.

The ethanol that is produced from these methods can be substantially pure. The ethanol produced can be recovered. Additionally, non-ethanol products (i.e., by-products) can also be recovered, such as acetaldehyde.

### Fatty (or Aliphatic Long Chain) Alcohol

Disclosed herein is a method of making fatty (or aliphatic long chain) alcohol comprising contacting a genetically modified microorganism with a carbon substrate, where the microorganism comprises at least one heterologous gene under the control of a molecular switch encoding a fatty-acyl-CoA reductase (FAR). In some cases, the at least one heterologous gene is integrated into the genome of the microorganism. The method can further comprise growing the microorganism to produce fatty (or aliphatic long chain) alcohol. The FAR gene can be under the control of a switch, such as an inducible or repressible promoter that is responsive to the presence or absence of a component in the media, e.g., a sugar such as arabinose or a rare earth element such as lanthanum. Also the microorganism can be first grown in media that contains a rare earth metal (e.g., at least 1 μM lanthanum) and then subsequently the rare earth metal can be removed (e.g., by being diluted out of the media). This removal can occur before growing the microorganism to produce fatty (or aliphatic long chain) alcohol.

The fatty (or aliphatic long chain) alcohol that is produced from these methods can be substantially pure. The fatty (or aliphatic long chain) alcohol produced can be recovered. Additionally, non-fatty (or aliphatic long chain) alcohol products (i.e., by-products) can also be recovered.

### Fatty Acid Methyl Ester

Disclosed herein is a method of making fatty acid methyl ester comprising contacting a genetically modified microorganism with a carbon substrate, where the microorganism comprises at least one heterologous gene under the control of a molecular switch encoding a WES polypeptide. In some cases, the at least one heterologous gene is integrated into the genome of the microorganism. The method can further comprise growing the microorganism to produce fatty acid methyl ester. The WES polypeptide gene can be under the control of a switch, such as an inducible or repressible promoter that is responsive to the presence or absence of a component in the media, e.g., a sugar such as arabinose or a rare earth element such as lanthanum. Also the microorganism can be first grown in media that contains a rare earth metal (e.g., at least 1 μM lanthanum) and then subsequently the rare earth metal can be removed (e.g., by being diluted out of the media). This removal can occur before growing the microorganism to produce fatty acid methyl ester.

The fatty acid methyl ester that is produced from these methods can be substantially pure. The fatty acid methyl ester produced can be recovered. Additionally, non-fatty acid methyl ester products (i.e., by-products) can also be recovered.

### V. Fermentation

In general, the microorganisms disclosed herein should be used in fermentation conditions that are appropriate to convert a C1 carbon (such as methane) to 2,3-BDO; 1,4-BDO; isobutyraldehyde; isobutanol; 1-butanol; n-butanol; ethanol; fatty (or aliphatic long chain) alcohols; and fatty acid methyl esters (or other desired product). Reaction conditions that should be considered include temperature, media flow rate, pH, media redox potential, agitation rate (if using a continuous stirred tank reactor), inoculum level, maximum substrate concentrations and rates of introduction of the substrate to the bioreactor to ensure that substrate level does not become limiting, and maximum product concentrations to avoid product inhibition.

The optimum reaction conditions will depend partly on the particular microorganism of used. However, in some cases, it is preferred that the fermentation be performed at a pressure higher than ambient pressure. Operating at increased pressures can allow for a significant increase in the rate of C1-carbon transfer from the gas phase to the liquid phase where it can be taken up by the microorganism as a carbon source for the production of the multicarbon products. This in turn can mean that the retention time (defined as the liquid volume in the bioreactor divided by the input gas flow rate) can be reduced when bioreactors are maintained at elevated pressure rather than atmospheric pressure.

The use of pressurized systems can greatly reduce the volume of the bioreactor required, and consequently the capital cost of the fermentation equipment. In some cases, reactor volume can be reduced in linear proportion to increases in reactor operating pressure, i.e. bioreactors operated at 10 atmospheres of pressure need only be one tenth the volume of those operated at 1 atmosphere of pressure.

It is also desirable that the rate of introduction of the C1-carbon substrate (such as methane) containing gaseous substrate is such as to ensure that the concentration of the C1-carbon substrate (such as methane) in the liquid phase does not become limiting. This is because a consequence of C1-carbon substrate (e.g., methane) limited conditions may be that the multicarbon product is consumed by the culture.

### Fermentation Conditions

pH can be optimized based on the microorganism used. For example, the pH used during methanotroph fermentation of methane to a desired product (such as a multicarbon product), can be from 4 to 10. In other instances, the pH can be from 5 to 9; 6 to 8; 6.1 to 7.9; 6.2 to 7.8; 6.3 to 7.7; 6.4 to 7.6; or 6.5 to 7.5. For example, the pH can be from 6.6 to 7.4. In some cases, the pH can be from 5 to 9. In some cases, the pH can be from 6 to 8. In some cases, the pH can be from 6.1 to 7.9. In some cases, the pH can be from 6.2 to 7.8. In some cases, the pH can be from 6.3 to 7.7. In some cases, the pH can be from 6.4 to 7.6. In some cases, the pH can be from 6.5 to 7.5. In some cases the pH used for methanotroph fermentation can be greater than 6.

Temperature can also be adjusted based on the microorganism used. For example, the temperature used during methanotroph fermentation of methane to a desired product (such as a multicarbon product), can be from 30 C° to 45 C°. In other instances, the temperature of the fermentation can be from 30 C.° to 45 C.°; 31 C.° to 44 C.°; 32 C.° to 43 C.°; 33 C.° to 42 C.°; 34 C.° to 41 C.°; 35 C.° to 40 C.°. For example, the temperature can be from 36 C.° to 39 C.° (e.g., 36 C.°, 37 C.°, 38 C.°, or 39 C.°). In some cases, the temperature can be from 30 C.° to 45 C.° (e.g., 30 C.°, 31 C.°, 32 C.°, 33 C.°, 34 C.°, 35 C.°, 36 C.°, 37 C.°, 38 C.°, 39 C.°, 40 C.°, 41 C.°, 42 C.°, 43 C.°, 44 C.°, or 45 C.°). In some cases, the temperature can be from 31 C.° to 44 C.° (e.g., 31 C.°, 32 C.°, 33 C.°, 34 C.°, 35 C.°, 36 C.°, 37 C.°, 38 C.°, 39 C.°, 40 C.°, 41 C.°, 42 C.°, 43 C.°, or 44 C.°). In some cases, the temperature can be from 32 C.° to 43 C.°. In some cases, the temperature can be from 33 C.° to 42 C.° (e.g., 33 C.°, 34 C.°, 35 C.°, 36 C.°, 37 C.°, 38 C.°, 39 C.°, 40 C.°, 41 C.°, or 42 C.°). In some cases, the temperature can be from 34 C.° to 41 C.° (e.g., 34 C.°, 35 C.°, 36 C.°, 37 C.°, 38 C.°, 39 C.°, 40 C.°, or 41 C.°). In some cases, the temperature can be from 35 C.° to 40 C.° (e.g., 35 C.°, 36 C.°, 37 C.°, 38 C.°, 39 C.°, or 40 C.°).

In some cases, the temperature of fermentation can be from 37.0 C.° to 47.9 C.°. In some cases, the temperature of fermentation can be from 37.1 C.° to 47.8 C.°. In some cases, the temperature of fermentation can be from 37.2 C.° to 47.7 C.°. In some cases, the temperature of fermentation can be from 37.3 C.° to 47.6 C.°. In some cases, the temperature of fermentation can be from 37.4 C.° to 47.5 C.°. In some cases, the temperature of fermentation can be from 37.5 C.° to 47.4 C.°. In some cases, the temperature of fermentation can be from 37.6 C.° to 47.3 C.°. In some cases, the temperature of fermentation can be from 37.7 C.° to 47.2 C.°. In some cases, the temperature of fermentation can be from 37.8 C.° to 47.1 C.°. In some cases, the temperature of fermentation can be from 37.9 C.° to 47.0 C.°. In some cases, the temperature of fermentation can be from 38.0 C.° to 46.9 C.°. In some cases, the temperature of fermentation can be from 38.1 C.° to 46.8 C.°. In some cases, the temperature of fermentation can be from 38.2 C.° to 46.7 C.°. In some cases, the temperature of fermentation can be from 38.3 C.° to 46.6 C.°. In some cases, the temperature of fermentation can be from 38.4 C.° to 46.5 C.°. In some cases, the temperature of fermentation can be from 38.5 C.° to 46.4 C.°. In some cases, the temperature of fermentation can be from 38.6 C.° to 46.3 C.°. In some cases, the temperature of fermentation can be from 38.7 C.° to 46.2 C.°. In some cases, the temperature of fermentation can be from 38.8 C.° to 46.1 C.°. In some cases, the temperature of fermentation can be from 38.9 C.° to 46.0 C.°. In some cases, the temperature of fermentation can be from 39.0 C.° to 45.9 C.°. In some cases, the temperature of fermentation can be from 39.1 C.° to 45.8 C.°. In some cases, the temperature of fermentation can be from 39.2 C.° to 45.7 C.°. In some cases, the temperature of fermentation can be from 39.3 C.° to 45.6 C.°. In some cases, the temperature of fermentation can be from 39.4 C.° to 45.5 C.°. In some cases, the temperature of fermentation can be from 39.5 C.° to 45.4 C.°. In some cases, the temperature of fermentation can be from 39.6 C.° to 45.3 C.°. In some cases, the temperature of fermentation can be from 39.7 C.° to 45.2 C.°. In some cases, the temperature of fermentation can be from 39.8 C.° to 45.1 C.°. In some cases, the temperature of fermentation can be from 39.9 C.° to 45.0 C.°. In some cases, the temperature of fermentation can be from 40.0 C.° to 44.9 C.°. In some cases, the temperature of fermentation can be from 40.1 C.° to 44.8 C.°. In some cases, the temperature of fermentation can be from 40.2 C.° to 44.7 C.°. In some cases, the temperature of fermentation can be from 40.3 C.° to 44.6 C.°. In some cases, the temperature of fermentation can be from 40.4 C.° to 44.5 C.°. In some cases, the temperature of fermentation can be from 40.5 C.° to 44.4 C.°. In some cases, the temperature of fermentation can be from 40.6 C.° to 44.3 C.°. In some cases, the temperature of fermentation can be from 40.7 C.° to 44.2 C.°. In some cases, the temperature of fermentation can be from 40.8 C.° to 44.1 C.°. In some cases, the temperature of fermentation can be from 40.9 C.° to 44.0 C.°. In some cases, the temperature of fermentation can be from 41.0 C.° to 43.9 C.°. In some cases, the temperature of fermentation can be from 41.1 C.° to 43.8 C.°. In some cases, the temperature of fermentation can be from 41.2 C.° to 43.7 C.°. In some cases, the temperature of fermentation can be from 41.3 C.° to 43.6 C.°. In some cases, the temperature of fermentation can be from 41.4 C.° to 43.5 C.°. In some cases, the temperature of fermentation can be from 41.5 C.° to 43.4 C.°. In some cases, the temperature of fermentation can be from 41.6 C.° to 43.3 C.°. In some cases, the temperature of fermentation can be from 41.7 C.° to 43.2 C.°. In some cases, the temperature of fermentation can be from 41.8 C.° to 43.1 C.°. In some cases, the temperature of fermentation can be from 41.9 C.° to 43.0 C.°. In some cases, the temperature of fermentation can be from 42.0 C.° to 42.9 C.°. In some cases, the temperature of fermentation can be from 42.1 C.° to 42.8 C.°. In some cases, the temperature of fermentation can be from 42.2 C.° to 42.7 C.°. In some cases, the temperature of fermentation can be from 42.3 C.° to 42.6 C.°. In some cases, the temperature of fermentation can be from 42.4 C.° to 42.5 C.°.

Availability of oxygen and other gases such as gaseous C1-carbon substrates (such as methane) can affect yield and fermentation rate. For example, when considering oxygen availability, the percent of dissolved oxygen (DO) within the fermentation media can be from 1% to 40%. In certain instances, the DO concentration can be from 1.5% to 35%; 2% to 30%; 2.5% to 25%; 3% to 20%; 4% to 19%; 5% to 18%; 6% to 17%; 7% to 16%; 8% to 15%; 9% to 14%; 10% to 13%; or 11% to 12%. For example, in some cases the DO concentration can be from 2% to 30%. In other cases, the DO can be from 3% to 20%. In some cases, the DO can be from 4% to 10%. In some cases, the DO can be from 1.5% to 35%. In some cases, the DO can be from 2.5% to 25%. In some cases, the DO can be from 4% to 19%. In some cases, the DO can be from 5% to 18%. In some cases, the DO can be from 6% to 17%. In some cases, the DO can be from 7% to 16%. In some cases, the DO can be from 8% to 15%. In some cases, the DO can be from 9% to 14%. In some cases, the DO can be from 10% to 13%. In some cases, the DO can be from 11% to 12%.

When using a methanotroph, the type of methane substances can have an effect on yield and fermentation rates. For example, natural gas can be used, which typically has a methane content of above 85% (e.g., above 90%) methane. Other components within natural gas can include but is not limited to ethane, propane, iso-butane, normal-butane, iso-pentane, normal pentane, hexanes plus, nitrogen, carbon dioxide, oxygen, hydrogen, and hydrogen sulfides.

“Pure” methane can be used as well. In these cases, the methane typically comes from a tank. The methane contained within these tanks can range from 90% or greater methane content and the remaining gas are other gases (such as carbon dioxide). For example, gas having a methane content of greater than 90% can be used during the fermentation process. In certain instances, the methane concentration can be greater than 90%; 91%; 92%; 93%; 94%; 95%; 96%; 97%; 98%; 99%; or 99.9%. In some cases, the methane concentration can be 90% methane and 10% are other gases (such as carbon dioxide). In other instances, the methane concentration can be 91% methane and 9% are other gases (such as carbon dioxide). In some cases, the methane concentration can be 92% methane and 8% are other gases (such as carbon dioxide). In some cases, the methane concentration can be 93% methane and 7% are other gases (such as carbon dioxide). In some cases, the methane concentration can be 94% methane and 6% are other gases (such as carbon dioxide). In some cases, the methane concentration can be 95% methane and 5% are other gases (such as carbon dioxide). In other instances, the methane concentration can be 96% methane and 4% are other gases (such as carbon dioxide). In some cases, the methane concentration can be 97% methane and 3% are other gases (such as carbon dioxide). In some cases, the methane concentration can be 98% methane and 2% are other gases (such as carbon dioxide). In some cases, the methane concentration can be 99% methane and 1% are other gases (such as carbon dioxide). In some cases, the methane concentration can be 99.9% methane and 0.1% are other gases (such as carbon dioxide).

### Switches

Some switches can be used in order to repress expression. For example, a rare earth metal with the atomic number of less than 63 can be used to repress expression. In some cases, a rare earth metal with an atomic number of less than 62 can be used to repress expression. In some cases, a rare earth metal with an atomic number of less than 61 can be used. In some cases, a rare earth metal with an atomic number of less than 60 can be used to repress expression. Some switches can be used to activate expression. For example, in some cases, a rare earth metal with the atomic number of greater than 63 can be used to induce expression.

### Lanthanum

In cases where a switch is used, the media can comprise a molecule that induces or represses the switch. For example, when a lanthanum sensitive switch is used to repress the expression of one or more of the genes described herein, the media can comprise lanthanum, which will repress expression of the one or more genes under the control of the switch. In the case of lanthanum any one of the following concentrations can effectively repress expression of the one or more genes: 0.1 μM; 0.5 μM; 1 μM; 2 μM; 3 μM; 4 μM; 5 μM; 6 μM; 7 μM; 8 μM; 9 μM; 10 μM; 12.5 μM; 15 μM; 17.5 μM; 20 μM; 25 μM; 50 μM; 100 μM or more. In one case, 0.1 μM lanthanum can be used to repression expression of the one or more genes under the control of a lanthanum switch. In other cases, at least 0.5 μM lanthanum can be used. In other cases, at least 1 μM lanthanum can be used. In other cases, at least 2 μM lanthanum can be used. In other cases, at least 3 μM lanthanum can be used. In other cases, at least 4 μM lanthanum can be used. In other cases, at least 5 μM lanthanum can be used. In other cases, at least 6 μM lanthanum can be used. In other cases, at least 7 μM lanthanum can be used. In other cases, at least 8 μM lanthanum can be used. In other cases, at least 9 μM lanthanum can be used. In other cases, at least 10 μM lanthanum can be used. In other cases, at least 12.5 μM lanthanum can be used. In other cases, at least 15 μM lanthanum can be used. In other cases, at least 17.5 μM lanthanum can be used. In other cases, at least 20 μM lanthanum can be used. In other cases, at least 25 μM lanthanum can be used. In other cases, at least 50 μM lanthanum can be used. In other cases, at least 100 μM lanthanum can be used. In some cases, a range of 0.5 μM lanthanum to 100 μM lanthanum will effectively repress gene expression. In some cases, a range of 0.5 μM lanthanum to 50 μM lanthanum will repress gene expression. In other cases, a range of 1 μM lanthanum to 20 μM lanthanum will repress gene expression. In some cases, a range of 2 μM lanthanum to 15 μM lanthanum will repress gene expression. In some cases, a range of 3 μM lanthanum to 12.5 μM lanthanum will repress gene expression. In some cases, a range of 4 μM lanthanum to 12 μM lanthanum will repress gene expression. In some cases, a range of 5 μM lanthanum to 11.5 μM lanthanum will repress gene expression. In some cases, a range of 6 μM lanthanum to 11 μM lanthanum will repress gene expression. In some cases, a range of 7 μM lanthanum to 10.5 μM lanthanum will repress gene expression. In some cases, a range of 8 μM lanthanum to 10 μM lanthanum will repress gene expression. In some cases, the maximal repressive effect of lanthanum is from 10 μM to 35 μM of lanthanum. In some cases, the maximal repressive effective of lanthanum is at about 35 μM.

In some cases, the lanthanum in the media can be diluted to turn on expression of the one or more lanthanum repressed genes. For example, in some cases, the dilution of lanthanum containing media can be 1:1 (1 part lanthanum containing media to 1 part non-lanthanum containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

In some cases, the microorganism can be grown in media comprising lanthanum. The media can then be diluted to effectively turn on the expression of the lanthanum repressed genes. The microorganism can be then grown in conditions to promote the production of desired products, such as the multicarbon products (or others disclosed throughout).

### Cerium

In certain cases, a cerium sensitive switch can be used to repress or induce the expression of one or more of the genes described herein. In some cases, the media can comprise cerium, which can in some instances repress expression of the one or more genes under the control of the switch. In the case of cerium any one of the following concentrations can effectively repress expression of the one or more genes: 1 μM; 2 μM; 3 μM; 4 μM; 5 μM; 6 μM; 7 μM; 8 μM; 9 μM; 10 μM; 12.5 μM; 15 μM; 17.5 μM; 20 μM; 25 μM; 30 μM; 35 μM; 40 μM; 45 μM; 50 μM; 55 μM; 60 μM; 65 μM; 70 μM; 75 μM; 80 μM; 85 μM; 90 μM; 95 μM; 100 μM; 105 μM; 110 μM; 115 μM; 120 μM; 125 μM; 130 μM; 135 μM; 140 μM; 145 μM; 150 μM; 155 μM; 160 μM; 165 μM; 170 μM; 175 μM; 180 μM; 185 μM; 190 μM; 195 μM; 200 μM or more. In one case, at least 1 μM cerium can be used. In other cases, at least 2 μM cerium can be used. In other cases, at least 3 μM cerium can be used. In other cases, at least 4 μM cerium can be used. In other cases, at least 5 μM cerium can be used. In other cases, at least 6 μM cerium can be used. In other cases, at least 7 μM cerium can be used. In other cases, at least 8 μM cerium can be used. In other cases, at least 9 μM cerium can be used. In other cases, at least 10 μM cerium can be used. In other cases, at least 12.5 μM cerium can be used. In other cases, at least 15 μM cerium can be used. In other cases, at least 17.5 μM cerium can be used. In other cases, at least 20 μM cerium can be used. In other cases, at least 25 μM cerium can be used. In other cases, at least 25 μM cerium can be used. In other cases, at least 30 μM cerium can be used. In other cases, at least 35 μM cerium can be used. In other cases, at least 40 μM cerium can be used. In other cases, at least 45 μM cerium can be used. In other cases, at least 50 μM cerium can be used. In other cases, at least 55 μM cerium can be used. In other cases, at least 60 μM cerium can be used. In other cases, at least 65 μM cerium can be used. In other cases, at least 70 μM cerium can be used. In other cases, at least 75 μM cerium can be used. In other cases, at least 80 μM cerium can be used. In other cases, at least 85 μM cerium can be used. In other cases, at least 90 μM cerium can be used. In other cases, at least 95 μM cerium can be used. In other cases, at least 100 μM cerium can be used. In other cases, at least 105 μM cerium can be used. In other cases, at least 110 μM cerium can be used. In other cases, at least 115 μM cerium can be used. In other cases, at least 120 μM cerium can be used. In other cases, at least 125 μM cerium can be used. In other cases, at least 130 μM cerium can be used. In other cases, at least 135 μM cerium can be used. In other cases, at least 140 μM cerium can be used. In other cases, at least 145 μM cerium can be used. In other cases, at least 150 μM cerium can be used. In other cases, at least 155 μM cerium can be used. In other cases, at least 160 μM cerium can be used. In other cases, at least 165 μM cerium can be used. In other cases, at least 170 μM cerium can be used. In other cases, at least 175 μM cerium can be used. In other cases, at least 180 μM cerium can be used. In other cases, at least 185 μM cerium can be used. In other cases, at least 190 μM cerium can be used. In other cases, at least 195 μM cerium can be used. In other cases, at least 200 μM or more cerium can be used.

In some cases, a range of 1 μM cerium to 200 μM cerium will effectively repress gene expression. In some cases, a range of 5 μM cerium to 175 μM cerium will repress gene expression. In other cases, a range of 7.5 μM cerium to 150 μM cerium will repress gene expression. In some cases, a range of 10 μM cerium to 145 μM cerium will repress gene expression. In some cases, a range of 15 μM cerium to 140 μM cerium will repress gene expression. In some cases, a range of 20 μM cerium to 125 μM cerium will repress gene expression. In some cases, a range of 25 μM cerium to 100 μM cerium will repress gene expression. In some cases, a range of 30 μM cerium to 90 μM cerium will repress gene expression. In some cases, a range of 35 μM cerium to 75 μM cerium will repress gene expression. In some cases, a range of 40 μM cerium to 65 μM cerium will repress gene expression. In some cases, the maximal repressive effect of cerium is from 35 μM to 70 μM of cerium. In some cases, the maximal repressive effective of lanthanum is at about 70 μM.

In some cases, the cerium in the media can be diluted to turn on expression of the one or more cerium repressed genes. For example, in some cases, the dilution of cerium containing media can be 1:1 (1 part cerium containing media to 1 part non-cerium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

In some cases, the microorganism can be grown in media comprising cerium. The media can then be diluted to effectively turn on the expression of the cerium repressed genes. The microorganism can be then grown in conditions to promote the production of desired products, such as the multicarbon products (or others disclosed throughout).

### Praseodymium

In certain cases, a praseodymium switch can be used to induce or repress the expression of one or more of the genes described herein. In some cases, the media can comprise praseodymium, which will in certain instances repress expression of the one or more genes under the control of the switch. In the case of praseodymium any one of the following concentrations can effectively repress expression of the one or more genes: 1 μM; 2 μM; 3 μM; 4 μM; 5 μM; 6 μM; 7 μM; 8 μM; 9 μM; 10 μM; 12.5 μM; 15 μM; 17.5 μM; 20 μM; 25 μM; 30 μM; 35 μM; 40 μM; 45 μM; 50 μM; 55 μM; 60 μM; 65 μM; 70 μM; 75 μM; 80 μM; 85 μM; 90 μM; 95 μM; 100 μM; 105 μM; 110 μM; 115 μM; 120 μM; 125 μM; 130 μM; 135 μM; 140 μM; 145 μM; 150 μM; 155 μM; 160 μM; 165 μM; 170 μM; 175 μM; 180 μM; 185 μM; 190 μM; 195 μM; 200 μM or more. In one case, at least 1 μM praseodymium can be used. In other cases, at least 2 μM praseodymium can be used. In other cases, at least 3 μM praseodymium can be used. In other cases, at least 4 μM praseodymium can be used. In other cases, at least 5 μM praseodymium can be used. In other cases, at least 6 μM praseodymium can be used. In other cases, at least 7 μM praseodymium can be used. In other cases, at least 8 μM praseodymium can be used. In other cases, at least 9 μM praseodymium can be used. In other cases, at least 10 μM praseodymium can be used. In other cases, at least 12.5 μM praseodymium can be used. In other cases, at least 15 μM praseodymium can be used. In other cases, at least 17.5 μM praseodymium can be used. In other cases, at least 20 μM praseodymium can be used. In other cases, at least 25 μM praseodymium can be used. In other cases, at least 25 μM praseodymium can be used. In other cases, at least 30 μM praseodymium can be used. In other cases, at least 35 μM praseodymium can be used. In other cases, at least 40 μM praseodymium can be used. In other cases, at least 45 μM praseodymium can be used. In other cases, at least 50 μM praseodymium can be used. In other cases, at least 55 μM praseodymium can be used. In other cases, at least 60 μM praseodymium can be used. In other cases, at least 65 μM praseodymium can be used. In other cases, at least 70 μM praseodymium can be used. In other cases, at least 75 μM praseodymium can be used. In other cases, at least 80 μM praseodymium can be used. In other cases, at least 85 μM praseodymium can be used. In other cases, at least 90 μM praseodymium can be used. In other cases, at least 95 μM praseodymium can be used. In other cases, at least 100 μM praseodymium can be used. In other cases, at least 105 μM praseodymium can be used. In other cases, at least 110 μM praseodymium can be used. In other cases, at least 115 μM praseodymium can be used. In other cases, at least 120 μM praseodymium can be used. In other cases, at least 125 μM praseodymium can be used. In other cases, at least 130 μM praseodymium can be used. In other cases, at least 135 μM praseodymium can be used. In other cases, at least 140 μM praseodymium can be used. In other cases, at least 145 μM praseodymium can be used. In other cases, at least 150 μM praseodymium can be used. In other cases, at least 155 μM praseodymium can be used. In other cases, at least 160 μM praseodymium can be used. In other cases, at least 165 μM praseodymium can be used. In other cases, at least 170 μM praseodymium can be used. In other cases, at least 175 μM praseodymium can be used. In other cases, at least 180 μM praseodymium can be used. In other cases, at least 185 μM praseodymium can be used. In other cases, at least 190 μM praseodymium can be used. In other cases, at least 195 μM praseodymium can be used. In other cases, at least 200 μM or more praseodymium can be used.

In some cases, a range of 1 μM praseodymium to 200 μM praseodymium will effectively repress gene expression. In some cases, a range of 5 μM praseodymium to 175 μM praseodymium will repress gene expression. In other cases, a range of 7.5 μM praseodymium to 150 μM praseodymium will repress gene expression. In some cases, a range of 10 μM praseodymium to 145 μM praseodymium will repress gene expression. In some cases, a range of 15 μM praseodymium to 140 μM praseodymium will repress gene expression. In some cases, a range of 20 μM praseodymium to 125 μM praseodymium will repress gene expression. In some cases, a range of 25 μM praseodymium to 100 μM praseodymium will repress gene expression. In some cases, a range of 30 μM praseodymium to 90 μM praseodymium will repress gene expression. In some cases, a range of 35 μM praseodymium to 75 μM praseodymium will repress gene expression. In some cases, a range of 40 μM praseodymium to 65 μM praseodymium will repress gene expression. In some cases, the maximal repressive effect of praseodymium is at least 140 μM.

In some cases, the praseodymium in the media can be diluted to turn on expression of the one or more praseodymium repressed genes. For example, in some cases, the dilution of praseodymium containing media can be 1:1 (1 part praseodymium containing media to 1 part non-praseodymium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

In some cases, the microorganism can be grown in media comprising praseodymium. The media can then be diluted to effectively turn on the expression of the praseodymium repressed genes. The microorganism can be then grown in conditions to promote the production of desired products, such as the multicarbon products (or others disclosed throughout).

### Neodymium

In certain cases, a neodymium switch can be used to repress or induce the expression of one or more of the genes described herein. In some cases, the media can comprise neodymium, which will repress expression of the one or more genes under the control of the switch. In the case of neodymium any one of the following concentrations can effectively repress expression of the one or more genes: 1 μM; 2 μM; 3 μM; 4 μM; 5 μM; 6 μM; 7 μM; 8 μM; 9 μM; 10 μM; 12.5 μM; 15 μM; 17.5 μM; 20 μM; 25 μM; 30 μM; 35 μM; 40 μM; 45 μM; 50 μM; 55 μM; 60 μM; 65 μM; 70 μM; 75 μM; 80 μM; 85 μM; 90 μM; 95 μM; 100 μM; 105 μM; 110 μM; 115 μM; 120 μM; 125 μM; 130 μM; 135 μM; 140 μM; 145 μM; 150 μM; 155 μM; 160 μM; 165 μM; 170 μM; 175 μM; 180 μM; 185 μM; 190 μM; 195 μM; 200 μM or more. In one case, at least 1 μM neodymium can be used. In other cases, at least 2 μM neodymium can be used. In other cases, at least 3 μM neodymium can be used. In other cases, at least 4 μM neodymium can be used. In other cases, at least 5 μM neodymium can be used. In other cases, at least 6 μM neodymium can be used. In other cases, at least 7 μM neodymium can be used. In other cases, at least 8 μM neodymium can be used. In other cases, at least 9 μM neodymium can be used. In other cases, at least 10 μM neodymium can be used. In other cases, at least 12.5 μM neodymium can be used. In other cases, at least 15 μM neodymium can be used. In other cases, at least 17.5 μM neodymium can be used. In other cases, at least 20 μM neodymium can be used. In other cases, at least 25 μM neodymium can be used. In other cases, at least 25 μM neodymium can be used. In other cases, at least 30 μM neodymium can be used. In other cases, at least 35 μM neodymium can be used. In other cases, at least 40 μM neodymium can be used. In other cases, at least 45 μM neodymium can be used. In other cases, at least 50 μM neodymium can be used. In other cases, at least 55 μM neodymium can be used. In other cases, at least 60 μM neodymium can be used. In other cases, at least 65 μM neodymium can be used. In other cases, at least 70 μM neodymium can be used. In other cases, at least 75 μM neodymium can be used. In other cases, at least 80 μM neodymium can be used. In other cases, at least 85 μM neodymium can be used. In other cases, at least 90 μM neodymium can be used. In other cases, at least 95 μM neodymium can be used. In other cases, at least 100 μM neodymium can be used. In other cases, at least 105 μM neodymium can be used. In other cases, at least 110 μM neodymium can be used. In other cases, at least 115 μM neodymium can be used. In other cases, at least 120 μM neodymium can be used. In other cases, at least 125 μM neodymium can be used. In other cases, at least 130 μM neodymium can be used. In other cases, at least 135 μM neodymium can be used. In other cases, at least 140 μM neodymium can be used. In other cases, at least 145 μM neodymium can be used. In other cases, at least 150 μM neodymium can be used. In other cases, at least 155 μM neodymium can be used. In other cases, at least 160 μM neodymium can be used. In other cases, at least 165 μM neodymium can be used. In other cases, at least 170 μM neodymium can be used. In other cases, at least 175 μM neodymium can be used. In other cases, at least 180 μM neodymium can be used. In other cases, at least 185 μM neodymium can be used. In other cases, at least 190 μM neodymium can be used. In other cases, at least 195 μM neodymium can be used. In other cases, at least 200 μM or more neodymium can be used.

In some cases, a range of 1 μM neodymium to 200 μM neodymium will effectively repress gene expression. In some cases, a range of 5 μM neodymium to 175 μM neodymium will repress gene expression. In other cases, a range of 7.5 μM neodymium to 150 μM neodymium will repress gene expression. In some cases, a range of 10 μM neodymium to 145 μM neodymium will repress gene expression. In some cases, a range of 15 μM neodymium to 140 μM neodymium will repress gene expression. In some cases, a range of 20 μM neodymium to 125 μM neodymium will repress gene expression. In some cases, a range of 25 μM neodymium to 100 μM neodymium will repress gene expression. In some cases, a range of 30 μM neodymium to 90 μM neodymium will repress gene expression. In some cases, a range of 35 μM neodymium to 75 μM neodymium will repress gene expression. In some cases, a range of 40 μM neodymium to 65 μM neodymium will repress gene expression. In some cases, the maximal repressive effect of neodymium is at least 140 μM.

In some cases, the neodymium in the media can be diluted to turn on expression of the one or more neodymium repressed genes. For example, in some cases, the dilution of neodymium containing media can be 1:1 (1 part neodymium containing media to 1 part non-neodymium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

In some cases, the microorganism can be grown in media comprising neodymium. The media can then be diluted to effectively turn on the expression of the neodymium repressed genes. The microorganism can be then grown in conditions to promote the production of desired products, such as the multicarbon products (or others disclosed throughout).

### Scandium

In certain cases, a scandium switch can be used to repress or induce the expression of one or more of the genes described herein. In some cases, the media can comprise scandium, which will repress or induce expression of the one or more genes under the control of the switch. In the case of scandium any one of the following concentrations can effectively repress expression of the one or more genes: 10 μM; 20 μM; 30 μM; 40 μM; 50 μM; 60 μM; 70 μM; 80 μM; 90 μM; 100 μM; 120 μM; 140 μM; 150 μM; 175 μM; 200 μM or more. In other cases, at least 10 μM scandium can be used. In other cases, at least 20 μM scandium can be used. In other cases, at least 30 μM scandium can be used. In other cases, at least 40 μM scandium can be used. In other cases, at least 50 μM scandium can be used. In other cases, at least 60 μM scandium can be used. In other cases, at least 70 μM scandium can be used. In other cases, at least 80 μM scandium can be used. In other cases, at least 90 μM scandium can be used. In other cases, at least 100 μM scandium can be used. In other cases, at least 120 μM scandium can be used. In other cases, at least 140 μM scandium can be used. In other cases, at least 150 μM scandium can be used. In other cases, at least 175 μM scandium can be used. In other cases, at least 200 μM or more scandium can be used.

In some cases, a range of 10 μM scandium to 200 μM scandium will effectively repress gene expression. In some cases, a range of 20 μM scandium to 175 μM scandium will repress or induce gene expression. In other cases, a range of 30 μM scandium to 150 μM scandium will repress or induce gene expression. In some cases, a range of 40 μM scandium to 140 μM scandium will repress or induce gene expression. In some cases, a range of 50 μM scandium to 120 μM scandium will repress or induce gene expression. In some cases, a range of 60 μM scandium to 100 μM scandium will repress or induce gene expression. In some cases, a range of 70 μM scandium to 90 μM scandium will repress or induce gene expression.

In some cases, the scandium in the media can be diluted to reverse the effect of scandium. For example, in some cases, the dilution of scandium containing media can be 1:1 (1 part scandium containing media to 1 part non-scandium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

After dilution, the microorganism can be then grown in conditions to promote the production of desired products, such as the multicarbon products (or others disclosed throughout).

### Yttrium

In certain cases, a yttrium switch can be used to repress or induce the expression of one or more of the genes described herein. In some cases, the media can comprise yttrium, which will repress or induce expression of the one or more genes under the control of the switch. In the case of yttrium any one of the following concentrations can effectively repress expression of the one or more genes: 10 μM; 20 μM; 30 μM; 40 μM; 50 μM; 60 μM; 70 μM; 80 μM; 90 μM; 100 μM; 120 μM; 140 μM; 150 μM; 175 μM; 200 μM or more. In other cases, at least 10 μM yttrium can be used. In other cases, at least 20 μM yttrium can be used. In other cases, at least 30 μM yttrium can be used. In other cases, at least 40 μM yttrium can be used. In other cases, at least 50 μM yttrium can be used. In other cases, at least 60 μM yttrium can be used. In other cases, at least 70 μM yttrium can be used. In other cases, at least 80 μM yttrium can be used. In other cases, at least 90 μM yttrium can be used. In other cases, at least 100 μM yttrium can be used. In other cases, at least 120 μM yttrium can be used. In other cases, at least 140 μM yttrium can be used. In other cases, at least 150 μM yttrium can be used. In other cases, at least 175 μM yttrium can be used. In other cases, at least 200 μM or more yttrium can be used.

In some cases, a range of 10 μM yttrium to 200 μM yttrium will effectively repress gene expression. In some cases, a range of 20 μM yttrium to 175 μM yttrium will repress or induce gene expression. In other cases, a range of 30 μM yttrium to 150 μM yttrium will repress or induce gene expression. In some cases, a range of 40 μM yttrium to 140 μM yttrium will repress or induce gene expression. In some cases, a range of 50 μM yttrium to 120 μM yttrium will repress or induce gene expression. In some cases, a range of 60 μM yttrium to 100 μM yttrium will repress or induce gene expression. In some cases, a range of 70 μM yttrium to 90 μM yttrium will repress or induce gene expression.

In some cases, the yttrium in the media can be diluted to reverse the effect of yttrium. For example, in some cases, the dilution of yttrium containing media can be 1:1 (1 part yttrium containing media to 1 part non-yttrium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

After dilution, the microorganism can be then grown in conditions to promote the production of desired products, such as the multicarbon products (or others disclosed throughout).

### Samrium

In certain cases, a samrium switch can be used to repress or induce the expression of one or more of the genes described herein. In some cases, the media can comprise samrium, which will repress or induce expression of the one or more genes under the control of the switch. In the case of samrium any one of the following concentrations can effectively repress expression of the one or more genes: 10 μM; 20 μM; 30 μM; 40 μM; 50 μM; 60 μM; 70 μM; 80 μM; 90 μM; 100 μM; 120 μM; 140 μM; 150 μM; 175 μM; 200 μM or more. In other cases, at least 10 μM samrium can be used. In other cases, at least 20 μM samrium can be used. In other cases, at least 30 μM samrium can be used. In other cases, at least 40 μM samrium can be used. In other cases, at least 50 μM samrium can be used. In other cases, at least 60 μM samrium can be used. In other cases, at least 70 μM samrium can be used. In other cases, at least 80 μM samrium can be used. In other cases, at least 90 μM samrium can be used. In other cases, at least 100 μM samrium can be used. In other cases, at least 120 μM samrium can be used. In other cases, at least 140 μM samrium can be used. In other cases, at least 150 μM samrium can be used. In other cases, at least 175 μM samrium can be used. In other cases, at least 200 μM or more samrium can be used.

In some cases, a range of 10 μM samrium to 200 μM samrium will effectively repress or induce gene expression. In some cases, a range of 20 μM samrium to 175 μM samrium will repress or induce gene expression. In other cases, a range of 30 μM samrium to 150 μM samrium will repress or induce gene expression. In some cases, a range of 40 μM samrium to 140 μM samrium will repress or induce gene expression. In some cases, a range of 50 μM samrium to 120 μM samrium will repress or induce gene expression. In some cases, a range of 60 μM samrium to 100 μM samrium will repress or induce gene expression. In some cases, a range of 70 μM samrium to 90 μM samrium will repress or induce gene expression.

In some cases, the samrium in the media can be diluted to reverse the effect of samrium. For example, in some cases, the dilution of samrium containing media can be 1:1 (1 part samrium containing media to 1 part non-samrium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

After dilution, the microorganism can be then grown in conditions to promote the production of desired products, such as the multicarbon products (or others disclosed throughout).

### Europium

In certain cases, a europium switch can be used to repress or induce the expression of one or more of the genes described herein. In some cases, the media can comprise europium, which will repress or induce expression of the one or more genes under the control of the switch. In the case of europium any one of the following concentrations can effectively repress expression of the one or more genes: 10 μM; 20 μM; 30 μM; 40 μM; 50 μM; 60 μM; 70 μM; 80 μM; 90 μM; 100 μM; 120 μM; 140 μM; 150 μM; 175 μM; 200 μM or more. In other cases, at least 10 μM europium can be used. In other cases, at least 20 μM europium can be used. In other cases, at least 30 μM europium can be used. In other cases, at least 40 μM europium can be used. In other cases, at least 50 μM europium can be used. In other cases, at least 60 μM europium can be used. In other cases, at least 70 μM europium can be used. In other cases, at least 80 μM europium can be used. In other cases, at least 90 μM europium can be used. In other cases, at least 100 μM europium can be used. In other cases, at least 120 μM europium can be used. In other cases, at least 140 μM europium can be used. In other cases, at least 150 μM europium can be used. In other cases, at least 175 μM europium can be used. In other cases, at least 200 μM or more europium can be used.

In some cases, a range of 10 μM europium to 200 μM europium will effectively repress or induce gene expression. In some cases, a range of 20 μM europium to 175 μM europium will repress or induce gene expression. In other cases, a range of 30 μM europium to 150 μM europium will repress or induce gene expression. In some cases, a range of 40 μM europium to 140 μM europium will repress or induce gene expression. In some cases, a range of 50 μM europium to 120 μM europium will repress or induce gene expression. In some cases, a range of 60 μM europium to 100 μM europium will repress or induce gene expression. In some cases, a range of 70 μM europium to 90 μM europium will repress or induce gene expression.

In some cases, the europium in the media can be diluted to reverse the effect of europium. For example, in some cases, the dilution of europium containing media can be 1:1 (1 part europium containing media to 1 part non-europium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

After dilution, the microorganism can be then grown in conditions to promote the production of desired products, such as the multicarbon products (or others disclosed throughout).

### Gadolinium

In certain cases, a gadolinium switch can be used to repress or induce the expression of one or more of the genes described herein. In some cases, the media can comprise gadolinium, which will repress or induce expression of the one or more genes under the control of the switch. In the case of gadolinium any one of the following concentrations can effectively repress expression of the one or more genes: 10 μM; 20 μM; 30 μM; 40 μM; 50 μM; 60 μM; 70 μM; 80 μM; 90 μM; 100 μM; 120 μM; 140 μM; 150 μM; 175 μM; 200 μM or more. In other cases, at least 10 μM gadolinium can be used. In other cases, at least 20 μM gadolinium can be used. In other cases, at least 30 μM gadolinium can be used. In other cases, at least 40 μM gadolinium can be used. In other cases, at least 50 μM gadolinium can be used. In other cases, at least 60 μM gadolinium can be used. In other cases, at least 70 μM gadolinium can be used. In other cases, at least 80 μM gadolinium can be used. In other cases, at least 90 μM gadolinium can be used. In other cases, at least 100 μM gadolinium can be used. In other cases, at least 120 μM gadolinium can be used. In other cases, at least 140 μM gadolinium can be used. In other cases, at least 150 μM gadolinium can be used. In other cases, at least 175 μM gadolinium can be used. In other cases, at least 200 μM or more gadolinium can be used.

In some cases, a range of 10 μM gadolinium to 200 μM gadolinium will effectively repress or induce gene expression. In some cases, a range of 20 μM gadolinium to 175 μM gadolinium will repress or induce gene expression. In other cases, a range of 30 μM gadolinium to 150 μM gadolinium will repress or induce gene expression. In some cases, a range of 40 μM gadolinium to 140 μM gadolinium will repress or induce gene expression. In some cases, a range of 50 μM gadolinium to 120 μM gadolinium will repress or induce gene expression. In some cases, a range of 60 μM gadolinium to 100 μM gadolinium will repress or induce gene expression. In some cases, a range of 70 μM gadolinium to 90 μM gadolinium will repress or induce gene expression.

In some cases, the gadolinium in the media can be diluted to reverse the effect of gadolinium. For example, in some cases, the dilution of gadolinium containing media can be 1:1 (1 part gadolinium containing media to 1 part non-gadolinium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

After dilution, the microorganism can be then grown in conditions to promote the production of desired products, such as the multicarbon products (or others disclosed throughout).

### Terbium

In certain cases, a terbium switch can be used to repress or induce the expression of one or more of the genes described herein. In some cases, the media can comprise terbium, which will repress or induce expression of the one or more genes under the control of the switch. In the case of terbium any one of the following concentrations can effectively repress expression of the one or more genes: 10 μM; 20 μM; 30 μM; 40 μM; 50 μM; 60 μM; 70 μM; 80 μM; 90 μM; 100 μM; 120 μM; 140 μM; 150 μM; 175 μM; 200 μM or more. In other cases, at least 10 μM terbium can be used. In other cases, at least 20 μM terbium can be used. In other cases, at least 30 μM terbium can be used. In other cases, at least 40 μM terbium can be used. In other cases, at least 50 μM terbium can be used. In other cases, at least 60 μM terbium can be used. In other cases, at least 70 μM terbium can be used. In other cases, at least 80 μM terbium can be used. In other cases, at least 90 μM terbium can be used. In other cases, at least 100 μM terbium can be used. In other cases, at least 120 μM terbium can be used. In other cases, at least 140 μM terbium can be used. In other cases, at least 150 μM terbium can be used. In other cases, at least 175 μM terbium can be used. In other cases, at least 200 μM or more terbium can be used.

In some cases, a range of 10 μM terbium to 200 μM terbium will effectively repress or induce gene expression. In some cases, a range of 20 μM terbium to 175 μM terbium will repress or induce gene expression. In other cases, a range of 30 μM terbium to 150 μM terbium will repress or induce gene expression. In some cases, a range of 40 μM terbium to 140 μM terbium will repress or induce gene expression. In some cases, a range of 50 μM terbium to 120 μM terbium will repress or induce gene expression. In some cases, a range of 60 μM terbium to 100 μM terbium will repress or induce gene expression. In some cases, a range of 70 μM terbium to 90 μM terbium will repress or induce gene expression.

In some cases, the terbium in the media can be diluted to reverse the effect of terbium. For example, in some cases, the dilution of terbium containing media can be 1:1 (1 part terbium containing media to 1 part non-terbium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

After dilution, the microorganism can be then grown in conditions to promote the production of desired products, such as the multicarbon products (or others disclosed throughout).

### Dysprosium

In certain cases, a dysprosium switch can be used to repress or induce the expression of one or more of the genes described herein. In some cases, the media can comprise dysprosium, which will repress or induce expression of the one or more genes under the control of the switch. In the case of dysprosium any one of the following concentrations can effectively repress expression of the one or more genes: 10 μM; 20 μM; 30 μM; 40 μM; 50 μM; 60 μM; 70 μM; 80 μM; 90 μM; 100 μM; 120 μM; 140 μM; 150 μM; 175 μM; 200 μM or more. In other cases, at least 10 μM dysprosium can be used. In other cases, at least 20 μM dysprosium can be used. In other cases, at least 30 μM dysprosium can be used. In other cases, at least 40 μM dysprosium can be used. In other cases, at least 50 μM dysprosium can be used. In other cases, at least 60 μM dysprosium can be used. In other cases, at least 70 μM dysprosium can be used. In other cases, at least 80 μM dysprosium can be used. In other cases, at least 90 μM dysprosium can be used. In other cases, at least 100 μM dysprosium can be used. In other cases, at least 120 μM dysprosium can be used. In other cases, at least 140 μM dysprosium can be used. In other cases, at least 150 μM dysprosium can be used. In other cases, at least 175 μM dysprosium can be used. In other cases, at least 200 μM or more dysprosium can be used.

In some cases, a range of 10 μM dysprosium to 200 μM dysprosium will effectively repress or induce gene expression. In some cases, a range of 20 μM dysprosium to 175 μM dysprosium will repress or induce gene expression. In other cases, a range of 30 μM dysprosium to 150 μM dysprosium will repress or induce gene expression. In some cases, a range of 40 μM dysprosium to 140 μM dysprosium will repress or induce gene expression. In some cases, a range of 50 μM dysprosium to 120 μM dysprosium will repress or induce gene expression. In some cases, a range of 60 μM dysprosium to 100 μM dysprosium will repress or induce gene expression. In some cases, a range of 70 μM dysprosium to 90 μM dysprosium will repress or induce gene expression.

In some cases, the dysprosium in the media can be diluted to reverse the effect of dysprosium. For example, in some cases, the dilution of dysprosium containing media can be 1:1 (1 part dysprosium containing media to 1 part non-dysprosium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

After dilution, the microorganism can be then grown in conditions to promote the production of desired products, such as the multicarbon products (or others disclosed throughout).

### Holmium

In certain cases, a holmium switch can be used to repress or induce the expression of one or more of the genes described herein. In some cases, the media can comprise holmium, which will repress or induce expression of the one or more genes under the control of the switch. In the case of holmium any one of the following concentrations can effectively repress expression of the one or more genes: 10 μM; 20 μM; 30 μM; 40 μM; 50 μM; 60 μM; 70 μM; 80 μM; 90 μM; 100 μM; 120 μM; 140 μM; 150 μM; 175 μM; 200 μM or more. In other cases, at least 10 μM holmium can be used. In other cases, at least 20 μM holmium can be used. In other cases, at least 30 μM holmium can be used. In other cases, at least 40 μM holmium can be used. In other cases, at least 50 μM holmium can be used. In other cases, at least 60 μM holmium can be used. In other cases, at least 70 μM holmium can be used. In other cases, at least 80 μM holmium can be used. In other cases, at least 90 μM holmium can be used. In other cases, at least 100 μM holmium can be used. In other cases, at least 120 μM holmium can be used. In other cases, at least 140 μM holmium can be used. In other cases, at least 150 μM holmium can be used. In other cases, at least 175 μM holmium can be used. In other cases, at least 200 μM or more holmium can be used.

In some cases, a range of 10 μM holmium to 200 μM holmium will effectively repress or induce gene expression. In some cases, a range of 20 μM holmium to 175 μM holmium will repress or induce gene expression. In other cases, a range of 30 μM holmium to 150 μM holmium will repress or induce gene expression. In some cases, a range of 40 μM holmium to 140 μM holmium will repress or induce gene expression. In some cases, a range of 50 μM holmium to 120 μM holmium will repress or induce gene expression. In some cases, a range of 60 μM holmium to 100 μM holmium will repress or induce gene expression. In some cases, a range of 70 μM holmium to 90 μM holmium will repress or induce gene expression.

In some cases, the holmium in the media can be diluted to reverse the effect of holmium. For example, in some cases, the dilution of holmium containing media can be 1:1 (1 part holmium containing media to 1 part non-holmium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

After dilution, the microorganism can be then grown in conditions to promote the production of desired products, such as the multicarbon products (or others disclosed throughout).

### Erbium

In certain cases, an erbium switch can be used to repress or induce the expression of one or more of the genes described herein. In some cases, the media can comprise erbium, which will repress or induce expression of the one or more genes under the control of the switch. In the case of erbium any one of the following concentrations can effectively repress expression of the one or more genes: 10 μM; 20 μM; 30 μM; 40 μM; 50 μM; 60 μM; 70 μM; 80 μM; 90 μM; 100 μM; 120 μM; 140 μM; 150 μM; 175 μM; 200 μM or more. In other cases, at least 10 μM erbium can be used. In other cases, at least 20 μM erbium can be used. In other cases, at least 30 μM erbium can be used. In other cases, at least 40 μM erbium can be used. In other cases, at least 50 μM erbium can be used. In other cases, at least 60 μM erbium can be used. In other cases, at least 70 μM erbium can be used. In other cases, at least 80 μM erbium can be used. In other cases, at least 90 μM erbium can be used. In other cases, at least 100 μM erbium can be used. In other cases, at least 120 μM erbium can be used. In other cases, at least 140 μM erbium can be used. In other cases, at least 150 μM erbium can be used. In other cases, at least 175 μM erbium can be used. In other cases, at least 200 μM or more erbium can be used.

In some cases, a range of 10 μM erbium to 200 μM erbium will effectively repress or induce gene expression. In some cases, a range of 20 μM erbium to 175 μM erbium will repress or induce gene expression. In other cases, a range of 30 μM erbium to 150 μM erbium will repress or induce gene expression. In some cases, a range of 40 μM erbium to 140 μM erbium will repress or induce gene expression. In some cases, a range of 50 μM erbium to 120 μM erbium will repress or induce gene expression. In some cases, a range of 60 μM erbium to 100 μM erbium will repress or induce gene expression. In some cases, a range of 70 μM erbium to 90 μM erbium will repress or induce gene expression.

In some cases, the erbium in the media can be diluted to reverse the effect of erbium. For example, in some cases, the dilution of erbium containing media can be 1:1 (1 part erbium containing media to 1 part non-erbium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

After dilution, the microorganism can be then grown in conditions to promote the production of desired products, such as the multicarbon products (or others disclosed throughout).

### Thulium

In certain cases, a thulium switch can be used to repress or induce the expression of one or more of the genes described herein. In some cases, the media can comprise thulium, which will repress or induce expression of the one or more genes under the control of the switch. In the case of thulium any one of the following concentrations can effectively repress expression of the one or more genes: 10 μM; 20 μM; 30 μM; 40 μM; 50 μM; 60 μM; 70 μM; 80 μM; 90 μM; 100 μM; 120 μM; 140 μM; 150 μM; 175 μM; 200 μM or more. In other cases, at least 10 μM thulium can be used. In other cases, at least 20 μM thulium can be used. In other cases, at least 30 μM thulium can be used. In other cases, at least 40 μM thulium can be used. In other cases, at least 50 μM thulium can be used. In other cases, at least 60 μM thulium can be used. In other cases, at least 70 μM thulium can be used. In other cases, at least 80 μM thulium can be used. In other cases, at least 90 μM thulium can be used. In other cases, at least 100 μM thulium can be used. In other cases, at least 120 μM thulium can be used. In other cases, at least 140 μM thulium can be used. In other cases, at least 150 μM thulium can be used. In other cases, at least 175 μM thulium can be used. In other cases, at least 200 μM or more thulium can be used.

In some cases, a range of 10 μM thulium to 200 μM thulium will effectively repress or induce gene expression. In some cases, a range of 20 μM thulium to 175 μM thulium will repress or induce gene expression. In other cases, a range of 30 μM thulium to 150 μM thulium will repress or induce gene expression. In some cases, a range of 40 μM thulium to 140 μM thulium will repress or induce gene expression. In some cases, a range of 50 μM thulium to 120 μM thulium will repress or induce gene expression. In some cases, a range of 60 μM thulium to 100 μM thulium will repress or induce gene expression. In some cases, a range of 70 μM thulium to 90 μM thulium will repress or induce gene expression.

In some cases, the thulium in the media can be diluted to reverse the effect of thulium. For example, in some cases, the dilution of thulium containing media can be 1:1 (1 part thulium containing media to 1 part non-thulium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

After dilution, the microorganism can be then grown in conditions to promote the production of desired products, such as the multicarbon products (or others disclosed throughout).

### Yterrbium

In certain cases, a yterrbium switch can be used to repress or induce the expression of one or more of the genes described herein. In some cases, the media can comprise yterrbium, which will repress or induce expression of the one or more genes under the control of the switch. In the case of yterrbium any one of the following concentrations can effectively repress expression of the one or more genes: 10 μM; 20 μM; 30 μM; 40 μM; 50 μM; 60 μM; 70 μM; 80 μM; 90 μM; 100 μM; 120 μM; 140 μM; 150 μM; 175 μM; 200 μM or more. In other cases, at least 10 μM yterrbium can be used. In other cases, at least 20 μM yterrbium can be used. In other cases, at least 30 μM yterrbium can be used. In other cases, at least 40 μM yterrbium can be used. In other cases, at least 50 μM yterrbium can be used. In other cases, at least 60 μM yterrbium can be used. In other cases, at least 70 μM yterrbium can be used. In other cases, at least 80 μM yterrbium can be used. In other cases, at least 90 μM yterrbium can be used. In other cases, at least 100 μM yterrbium can be used. In other cases, at least 120 μM yterrbium can be used. In other cases, at least 140 μM yterrbium can be used. In other cases, at least 150 μM yterrbium can be used. In other cases, at least 175 μM yterrbium can be used. In other cases, at least 200 μM or more yterrbium can be used.

In some cases, a range of 10 μM yterrbium to 200 μM yterrbium will effectively repress or induce gene expression. In some cases, a range of 20 μM yterrbium to 175 μM yterrbium will repress or induce gene expression. In other cases, a range of 30 μM yterrbium to 150 μM yterrbium will repress or induce gene expression. In some cases, a range of 40 μM yterrbium to 140 μM yterrbium will repress or induce gene expression. In some cases, a range of 50 μM yterrbium to 120 μM yterrbium will repress or induce gene expression. In some cases, a range of 60 μM yterrbium to 100 μM yterrbium will repress or induce gene expression. In some cases, a range of 70 μM yterrbium to 90 μM yterrbium will repress or induce gene expression.

In some cases, the yterrbium in the media can be diluted to reverse the effect of yterrbium. For example, in some cases, the dilution of yterrbium containing media can be 1:1 (1 part yterrbium containing media to 1 part non-yterrbium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

After dilution, the microorganism can be then grown in conditions to promote the production of desired products, such as the multicarbon products (or others disclosed throughout).

### Arabinose

In some cases, an arabinose sensitive switch can be used to induce/repress the expression of one or more of the genes described herein. In these cases, the media can comprise arabinose, which will in some cases, induce the expression of the one or more genes under the control of the switch. In the case of arabinose any one of the following concentrations can effectively induce/repress expression of the one or more genes: 0.1 g/L; 0.5 g/L; 1 g/L; 2 g/L; 3 g/L; 4 g/L; 5 g/L; 6 g/L; 7 g/L; 8 g/L; 9 g/L; 10 g/L; 11 g/L; 12 g/L; 13 g/L; 14 g/L; 15 g/L; 16 g/L; 17 g/L; 18 g/L; 19 g/L; g/L; 20 g/L; 25 g/L; 30 g/L; 35 g/L; 40 g/L; 45 g/L; 50 g/L; 55 g/L; 60 g/L; 65 g/L; 70 g/L; 75 g/L; 80 g/L; 85 g/L; 90 g/L; 95 g/L; 100 g/L or more. In one case, 0.1 g/L arabinose can be used to induce/repress expression of the one or more genes under the control of an arabinose switch. In other cases, at least 0.5 g/L arabinose can be used. In other cases, at least 1 g/L arabinose can be used. In other cases, at least 2 g/L arabinose can be used. In other cases, at least 3 g/L arabinose can be used. In other cases, at least 4 g/L arabinose can be used. In other cases, at least 5 g/L arabinose can be used. In other cases, at least 6 g/L arabinose can be used. In other cases, at least 7 g/L arabinose can be used. In other cases, at least 8 g/L arabinose can be used. In other cases, at least 9 g/L arabinose can be used. In other cases, at least 10 g/L arabinose can be used. In other cases, at least 11 g/L arabinose can be used. In other cases, at least 12 g/L arabinose can be used. In other cases, at least 13 g/L arabinose can be used. In other cases, at least 14 g/L arabinose can be used. In other cases, at least 15 g/L arabinose can be used. In other cases, at least 16 g/L arabinose can be used. In other cases, at least 17 g/L arabinose can be used. In other cases, at least 18 g/L arabinose can be used. In other cases, at least 19 g/L arabinose can be used. In other cases, at least 20 g/L arabinose can be used. In other cases, at least 25 g/L arabinose can be used. In other cases, at least 30 g/L arabinose can be used. In other cases, at least 35 g/L arabinose can be used. In other cases, at least 40 g/L arabinose can be used. In other cases, at least 45 g/L arabinose can be used. In other cases, at least 50 g/L arabinose can be used. In other cases, at least 55 g/L arabinose can be used. In other cases, at least 60 g/L arabinose can be used. In other cases, at least 65 g/L arabinose can be used. In other cases, at least 70 g/L arabinose can be used. In other cases, at least 75 g/L arabinose can be used. In other cases, at least 80 g/L arabinose can be used. In other cases, at least 85 g/L arabinose can be used. In other cases, at least 90 g/L arabinose can be used. In other cases, at least 95 g/L arabinose can be used. In other cases, at least 100 g/L arabinose can be used. In some cases, a range of 0.5 g/L arabinose to 100 g/L arabinose will effectively induce/repress gene expression. In some cases, a range of 0.5 g/L arabinose to 50 g/L arabinose will induce/repress gene expression. In other cases, a range of 1 g/L arabinose to 20 g/L arabinose will induce/repress gene expression. In some cases, a range of 2 g/L arabinose to 15 g/L arabinose will induce/repress gene expression. In some cases, a range of 3 g/L arabinose to 12.5 g/L arabinose will induce/repress gene expression. In some cases, a range of 4 g/L arabinose to 12 g/L arabinose will induce/repress gene expression. In some cases, a range of 5 g/L arabinose to 11.5 g/L arabinose will induce/repress gene expression. In some cases, a range of 6 g/L arabinose to 11 g/L arabinose will induce/repress gene expression. In some cases, a range of 7 g/L arabinose to 10.5 g/L arabinose will induce/repress gene expression. In some cases, a range of 8 g/L arabinose to 10 g/L arabinose will induce/repress gene expression.

In some cases, any one of the following concentrations of arabinose can effectively induce/repress gene expression of the one or more genes: 0.1 mM; 0.2 mM; 0.3 mM; 0.4 mM; 0.5 mM; 0.6 mM; 0.7 mM; 0.8 mM; 0.9 mM; 1 mM; 1.5 mM; 2 mM; 2.5 mM; 3 mM; 3.5 mM; 4 mM; 4.5 mM; 5 mM; 5.5 mM; 6 mM; 6.6 mM; 7 mM; 7.5 mM; 8 mM; 8.5 mM; 9 mM; 9.5 mM; 10 mM; 12.5 mM; 15 mM; 17.5 mM; 20 mM; 25 mM; 50 mM; 100 mM or more. In one case, 0.1 mM arabinose can be used to repress/induce expression of the one or more genes under the control of an arabinose switch. In other cases, at least 0.2 mM arabinose can be used. In other cases, at least 0.3 mM arabinose can be used. In other cases, at least 0.4 mM arabinose can be used. In other cases, at least 0.5 mM arabinose can be used. In other cases, at least 0.6 mM arabinose can be used. In other cases, at least 0.7 mM arabinose can be used. In other cases, at least 0.8 mM arabinose can be used. In other cases, at least 0.9 mM arabinose can be used. In other cases, at least 1 mM arabinose can be used. In other cases, at least 1.5 mM arabinose can be used. In other cases, at least 2 mM arabinose can be used. In other cases, at least 2.5 mM arabinose can be used. In other cases, at least 3 mM arabinose can be used. In other cases, at least 3.5 mM arabinose can be used. In other cases, at least 4 mM arabinose can be used. In other cases, at least 4.5 mM arabinose can be used. In other cases, at least 5 mM arabinose can be used. In other cases, at least 5.5 mM arabinose can be used. In other cases, at least 6 mM arabinose can be used. In other cases, at least 6.6 mM arabinose can be used. In other cases, at least 7 mM arabinose can be used. In other cases, at least 7.5 mM arabinose can be used. In other cases, at least 8 mM arabinose can be used. In other cases, at least 8.5 mM arabinose can be used. In other cases, at least 9 mM arabinose can be used. In other cases, at least 9.5 mM arabinose can be used. In other cases, at least 10 mM arabinose can be used. In other cases, at least 12.5 mM arabinose can be used. In other cases, at least 15 mM arabinose can be used. In other cases, at least 17.5 mM arabinose can be used. In other cases, at least 20 mM arabinose can be used. In other cases, at least 25 mM arabinose can be used. In other cases, at least 50 mM arabinose can be used. In other cases, at least 100 mM arabinose can be used. In some cases, a range of 0.5 mM arabinose to 100 mM arabinose will effectively induce gene expression. In some cases, a range of 0.5 mM arabinose to 50 mM arabinose will induce gene expression. In other cases, a range of 1 mM arabinose to 20 mM arabinose will induce gene expression. In some cases, a range of 2 mM arabinose to 15 mM arabinose will induce gene expression. In some cases, a range of 3 mM arabinose to 12.5 mM arabinose will induce gene expression. In some cases, a range of 4 mM arabinose to 12 mM arabinose will induce gene expression. In some cases, a range of 5 mM arabinose to 11.5 mM arabinose will induce gene expression. In some cases, a range of 6 mM arabinose to 11 mM arabinose will induce gene expression. In some cases, a range of 7 mM arabinose to 10.5 mM arabinose will induce gene expression. In some cases, a range of 8 mM arabinose to 10 mM arabinose will induce gene expression.

In some cases, the arabinose in the media can be diluted to turn on/off the expression of the one or more arabinose repressed/induced genes. For example, in some cases, the dilution of arabinose containing media can be 1:1 (1 part arabinose containing media to 1 part non-arabinose containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

### Isopropyl β-D-1-thiogalactopyranoside

In certain cases, an IPTG sensitive switch can be used to induce or repress the expression of one or more of the genes described herein. In some cases, the media can comprise IPTG, which can in some instances induce expression of the one or more genes under the control of the switch. In the case of IPTG any one of the following concentrations can effectively induce or repress expression of the one or more genes: 1 μM; 2 μM; 3 μM; 4 μM; 5 μM; 6 μM; 7 μM; 8 μM; 9 μM; 10 μM; 12.5 μM; 15 μM; 17.5 μM; 20 μM; 25 μM; 30 μM; 35 μM; 40 μM; 45 μM; 50 μM; 55 μM; 60 μM; 65 μM; 70 μM; 75 μM; 80 μM; 85 μM; 90 μM; 95 μM; 100 μM; 105 μM; 110 μM; 115 μM; 120 μM; 125 μM; 130 μM; 135 μM; 140 μM; 145 μM; 150 μM; 155 μM; 160 μM; 165 μM; 170 μM; 175 μM; 180 μM; 185 μM; 190 μM; 195 μM; 200 μM or more. In one case, at least 1 μM IPTG can be used. In other cases, at least 2 μM IPTG can be used. In other cases, at least 3 μM IPTG can be used. In other cases, at least 4 μM IPTG can be used. In other cases, at least 5 μM IPTG can be used. In other cases, at least 6 μM IPTG can be used. In other cases, at least 7 μM IPTG can be used. In other cases, at least 8 μM IPTG can be used. In other cases, at least 9 μM IPTG can be used. In other cases, at least 10 μM IPTG can be used. In other cases, at least 12.5 μM IPTG can be used. In other cases, at least 15 μM IPTG can be used. In other cases, at least 17.5 μM IPTG can be used. In other cases, at least 20 μM IPTG can be used. In other cases, at least 25 μM IPTG can be used. In other cases, at least 25 μM IPTG can be used. In other cases, at least 30 μM IPTG can be used. In other cases, at least 35 μM IPTG can be used. In other cases, at least 40 μM IPTG can be used. In other cases, at least 45 μM IPTG can be used. In other cases, at least 50 μM IPTG can be used. In other cases, at least 55 μM IPTG can be used. In other cases, at least 60 μM IPTG can be used. In other cases, at least 65 μM IPTG can be used. In other cases, at least 70 μM IPTG can be used. In other cases, at least 75 μM IPTG can be used. In other cases, at least 80 μM IPTG can be used. In other cases, at least 85 μM IPTG can be used. In other cases, at least 90 μM IPTG can be used. In other cases, at least 95 μM IPTG can be used. In other cases, at least 100 μM IPTG can be used. In other cases, at least 105 μM IPTG can be used. In other cases, at least 110 μM IPTG can be used. In other cases, at least 115 μM IPTG can be used. In other cases, at least 120 μM IPTG can be used. In other cases, at least 125 μM IPTG can be used. In other cases, at least 130 μM IPTG can be used. In other cases, at least 135 μM IPTG can be used. In other cases, at least 140 μM IPTG can be used. In other cases, at least 145 μM IPTG can be used. In other cases, at least 150 μM IPTG can be used. In other cases, at least 155 μM IPTG can be used. In other cases, at least 160 μM IPTG can be used. In other cases, at least 165 μM IPTG can be used. In other cases, at least 170 μM IPTG can be used. In other cases, at least 175 μM IPTG can be used. In other cases, at least 180 μM IPTG can be used. In other cases, at least 185 μM IPTG can be used. In other cases, at least 190 μM IPTG can be used. In other cases, at least 195 μM IPTG can be used. In other cases, at least 200 μM or more IPTG can be used.

In some cases, a range of 1 μM IPTG to 200 μM IPTG will effectively induce or repress gene expression. In some cases, a range of 5 μM IPTG to 175 μM IPTG will induce or repress gene expression. In other cases, a range of 7.5 μM IPTG to 150 μM IPTG will induce or repress gene expression. In some cases, a range of 10 μM IPTG to 145 μM IPTG will induce or repress gene expression. In some cases, a range of 15 μM IPTG to 140 μM IPTG will induce or repress gene expression. In some cases, a range of 20 μM IPTG to 125 μM IPTG will induce or repress gene expression. In some cases, a range of 25 μM IPTG to 100 μM IPTG will induce or repress gene expression. In some cases, a range of 30 μM IPTG to 90 μM IPTG will induce or repress gene expression. In some cases, a range of 35 μM IPTG to 75 μM IPTG will induce or repress gene expression. In some cases, a range of 40 μM IPTG to 65 μM IPTG will induce or repress gene expression.

In some cases, the IPTG in the media can be diluted to turn on or off expression of the one or more IPTG induced or repressed genes. For example, in some cases, the dilution of IPTG containing media can be 1:1 (1 part IPTG containing media to 1 part non-IPTG containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

In some cases, the microorganism can be grown in media comprising IPTG. IPTG can be added to the media to turn on the expression of IPTG induced genes. The microorganism can be then grown in conditions to promote the production of desired products, such as the multicarbon products (or others disclosed throughout). The media can then be diluted to effectively turn off the expression of the IPTG induced genes.

### Bioreactor

Fermentation reactions may be carried out in any suitable bioreactor. In some cases, the bioreactor may comprise a first, growth reactor in which the microorganisms are cultured, and a second, fermentation reactor, to which broth from the growth reactor is fed and in which most of the fermentation product is produced.

### Product Recovery

The fermentation of the microorganisms disclosed herein can produce a broth comprising a desired product (e.g., a multicarbon product), one or more by-products, and/or the microorganism itself (e.g., a genetically modified methanotroph).

The microorganisms and the methods herein can produce multicarbon products such as 2,3-BDO; 1,4-BDO; isobutyraldehyde; isobutanol; 1-butanol; n-butanol; ethanol; fatty (or aliphatic long chain) alcohols; and/or fatty acid methyl esters at surprisingly high efficiency, more so than other known fermentation processes. For example, the microorganisms and the methods disclosed herein can convert a C1-carbon substrate (such as methane) at a rate of greater than 50%. This means that at least 50% of the C1-carbons within the systems are converted into product, such as 2,3-BDO; 1,4-BDO; isobutyraldehyde; isobutanol; 1-butanol; n-butanol; ethanol; fatty (or aliphatic long chain) alcohols; and/or fatty acid methyl esters. In some cases, the conversion of a C1-carbon substrate into multicarbon products can be at least 60%, 70%, 80%, 81%, 83%, 84%, 85%, 86%, 87%, 88%, 89%, 90%, 91%, 93%, 94%, 95%, 96%, 97%, 98%, or 99%. In some cases, the conversion of a C1-carbon substrate into multicarbon products can be at least 60%. In some cases, the conversion of a C1-carbon substrate into multicarbon products can be at least 70%. In some cases, the conversion of a C1-carbon substrate into multicarbon products can be at least 80%. In some cases, the conversion of a C1-carbon substrate into multicarbon products can be at least 81%. In some cases, the conversion of a C1-carbon substrate into multicarbon products can be at least 82%. In some cases, the conversion of a C1-carbon substrate into multicarbon products can be at least 83%. In some cases, the conversion of a C1-carbon substrate into multicarbon products can be at least 84%. In some cases, the conversion of a C1-carbon substrate into multicarbon products can be at least 85%. In some cases, the conversion of a C1-carbon substrate into multicarbon products can be at least 86%. In some cases, the conversion of a C1-carbon substrate into multicarbon products can be at least 87%. In some cases, the conversion of a C1-carbon substrate into multicarbon products can be at least 88%. In some cases, the conversion of a C1-carbon substrate into multicarbon products can be at least 89%. In some cases, the conversion of a C1-carbon substrate into multicarbon products can be at least 90%. In some cases, the conversion of a C1-carbon substrate into multicarbon products can be at least 91%. In some cases, the conversion of a C1-carbon substrate into multicarbon products can be at least 92%. In some cases, the conversion of a C1-carbon substrate into multicarbon products can be at least 93%. In some cases, the conversion of a C1-carbon substrate into multicarbon products can be at least 94%. In some cases, the conversion of a C1-carbon substrate into multicarbon products can be at least 95%. In some cases, the conversion of a C1-carbon substrate into multicarbon products can be at least 96%. In some cases, the conversion of a C1-carbon substrate into multicarbon products can be at least 97%. In some cases, the conversion of a C1-carbon substrate into multicarbon products can be at least 98%. In some cases, the conversion of a C1-carbon substrate into multicarbon products can be at least 99%.

In certain methods when producing multicarbon products, the concentration of multicarbon products in the fermentation broth is at least 1g/L. For example, the concentration of multicarbon products produced in the fermentation broth can be from 1 g/L to 5 g/L, 2 g/L to 6 g/L, 3 g/L to 7 g/L, 4 g/L to 8 g/L, 5 g/L to 9 g/L, or 6 g/L to 10 g/L. In some cases, the concentration of multicarbon products can be at least 9g/L. In some cases, the concentration of multicarbon products can be from 1 g/L to 5 g/L. In some cases, the concentration of multicarbon products can be from 2 g/L to 6 g/L. In some cases, the concentration of multicarbon products can be from 3 g/L to 7 g/L. In some cases, the concentration of multicarbon products can be from 4 g/L to 8 g/L. In some cases, the concentration of multicarbon products can be from 5 g/L to 9 g/L. In some cases, the concentration of multicarbon products can be from 6 g/L to 10 g/L.

In other cases, when microorganisms are used that normally produce at least some of the same multicarbon product such as 2,3-BDO; 1,4-BDO; isobutyraldehyde; isobutanol; 1-butanol; n-butanol; ethanol; fatty (or aliphatic long chain) alcohols; and/or fatty acid methyl esters, after genetic modification and fermentation, the genetically modified microorganism can produce the same multicarbon product in concentrations that are at least 1.1× the amount that is normally produced. In some cases, the genetically modified microorganism can produce at least 2×, 3×, 4×, 5×, 6×, 7×, 8×, 9×, 10×, 15×, 20×, 25×, 30×, 35×, 40×, 45×, 50×, 60×, 70×, 80×, 90× or 100× the amount that is normally produced (e.g., produced by a microorganism that is unmodified and of the same species as the genetically modified microorganism). In some cases, the genetically modified microorganism can produce at least 2×, 3×, 4×, 5×, 10×, 25×, 50×, and or 100× the amount that is normally produced. In some cases, the genetically modified microorganism can produce at least 2× the amount that is normally produced. In some cases, the genetically modified microorganism can produce at least 3× the amount that is normally produced. In some cases, the genetically modified microorganism can produce at least 4× the amount that is normally produced. In some cases, the genetically modified microorganism can produce at least 5× the amount that is normally produced. In some cases, the genetically modified microorganism can produce at least 10× the amount that is normally produced. In some cases, the genetically modified microorganism can produce at least 25× the amount that is normally produced. In some cases, the genetically modified microorganism can produce at least 50× the amount that is normally produced. In some cases, the genetically modified microorganism can produce at least 100× the amount that is normally produced.

As discussed above, in certain cases the multicarbon product produced in the fermentation reaction is converted to a different product such as MEK, butene, butadiene, or other products directly from the fermentation broth. In other cases, the multicarbon product is first recovered from the fermentation broth before conversion to a different product.

In some cases, multicarbon product can be continuously removed from a portion of broth and recovered as purified. In particular cases, the recovery of the multicarbon product includes passing the removed portion of the broth containing the multicarbon product through a separation unit to separate the microorganisms (e.g., genetically modified methanotroph) from the broth, to produce a cell-free multicarbon product permeate, and returning the microorganisms to the bioreactor. The cell-free multicarbon product containing permeate may then can be stored or be used for subsequent conversion to a different desired product.

The recovering of the desired multicarbon product and/or one or more other products or by-products produced in the fermentation reaction can comprise continuously removing a portion of the broth and recovering separately the multicarbon product and one or more other products from the removed portion of the broth. In some cases, the recovery of the multicarbon product and/or one or more other products includes passing the removed portion of the broth containing the multicarbon product and/or one or more other products through a separation unit to separate microorganisms from the multicarbon product and/or one or more other products, to produce a cell-free multicarbon product and one or more other product-containing permeate, and returning the microorganisms to the bioreactor.

In the above cases, the recovery of the multicarbon product and one or more other products can include first removing the multicarbon product from the cell-free permeate followed by removing the one or more other products from the cell-free permeate. The cell-free permeate can also then returned to the bioreactor.

The multicarbon product, or a mixed product stream containing the multicarbon product, can be recovered from the fermentation broth. For example, methods that can be used can include but are not limited to, fractional distillation or evaporation, pervaporation, and extractive fermentation. Further examples include: recovery using steam from whole fermentation broths; reverse osmosis combined with distillation; liquid-liquid extraction techniques involving solvent extraction of the multicarbon product; aqueous two-phase extraction of the multicarbon product in PEG/dextran system; solvent extraction using alcohols or esters, e.g., ethyl acetate, tributylphosphate, diethyl ether, n-butanol, dodecanol, oleyl alcohol, and an ethanol/phosphate system; aqueous two-phase systems composed of hydrophilic solvents and inorganic salts. See generally, Voloch, M., et al., (1985) and U.S. Pat. Pub. Appl. No. 2012/0045807.

In some cases prior to exposure to solvent, the fermentation broth is dewatered by evaporation or both microfiltration and reverse osmosis because of the low partition coefficient and the low selectivity of the multicarbon product. Repulsive extraction or salting out using potassium chloride (KCl) or dehydrated K₂CO₃ has also been investigated on the recovery of multicarbon products such as 2,3-BDO (Syu, M. J., “Biological production of 2,3-butanediol, Appl Microbiol Biotechnol., 55(1):10-8 (2001)) like the salting-out effect of K2CO3 on extraction of butanol in acetone-O butanol-ethanol fermentation. The removal of water from the fermentation broth was also tested before salting out because the concentration of the multicarbon product, such as 2,3-BDO in the broth was too low to be salted out even if at a saturated KCl or K₂CO₃ solution. See generally, U.S. Pat. Pub. Appl. No. 2012/0045807.

A yet further example of a method to recover a multicarbon product such as 2,3-BDO is to react it with formaldehyde to form a formal under catalysis of acid. The multicarbon product such as 2,3-BDO formal is collected in the top oil phase and allowed to react with acid methanol to form multicarbon product such as 2,3-BDO and methylal. Methylal can be hydrolyzed to methanol and formaldehyde. See generally, U.S. Pat. Pub. Appl. No. 2012/0045807.

A further example, may be the use of ionic liquids to extract the multicarbon product such as ethanol/2,3-BDO from clarified broth. Ionic liquids can be tailored in many ways to change physical properties. An advantage of this approach is that ionic liquids are not volatile. Some are water sensitive but others are not.

Pervaporation or vacuum membrane distillation can be used for multicarbon product fermentation such as ethanol, butanol, and 2,3-BDO in water as an extract from the fermentation broth. A microporous polytetrafluoroethylene (PTFE) membrane is used in the integrated process, while a silicone membrane is usually used in pervaporative ethanol or butanol fermentations. See generally, U.S. Pat. Pub. Appl. No. 2012/0045807.

By-products such as acids including acetate and butyrate may also be recovered from the fermentation broth using known methods. For example, an adsorption system involving an activated charcoal filter or electrodialysis may be used.

In certain cases of the invention, the multicarbon product and other by-products are recovered from the fermentation broth by continuously removing a portion of the broth from the bioreactor, separating microbial cells from the broth (conveniently by filtration, for example), and recovering the multicarbon product and others such as alcohols and acids from the broth. Alcohols may conveniently be recovered for example by distillation, and acids may be recovered for example by adsorption on activated charcoal. The separated microbial cells are returned to the fermentation bioreactor. The cell-free permeate remaining after the alcohol(s) and acid(s) have been removed is also preferably returned to the fermentation bioreactor. Additional nutrients may be added to the cell-free permeate to replenish the nutrient medium before it is returned to the bioreactor.

Also, if the pH of the broth was adjusted during recovery of the multicarbon product and/or by-products, the pH should be re-adjusted to a similar pH to that of the broth in the fermentation bioreactor, before being returned to the bioreactor.

In certain cases, the multicarbon product is continuously recovered from the fermentation broth or bioreactor and fed directly for chemical conversion to one or more of butene, butadiene, and/or methyl ethyl ketone. For example, the multicarbon product may be fed directly through a conduit to one or more vessel suitable for chemical synthesis of one or more of downstream products such as butene, butadiene, and/or methyl ethyl ketone or other down-stream chemical products in the case of 2,3-BDO.

While some cases have been shown and described herein, such cases are provided by way of example only. Numerous variations, changes, and substitutions will now occur to those skilled in the art without departing from the invention. It should be understood that various alternatives to the cases of the invention described herein will be employed in practicing the invention.

### EXAMPLES

### Example 1: Lanthanum Effect on Promoters

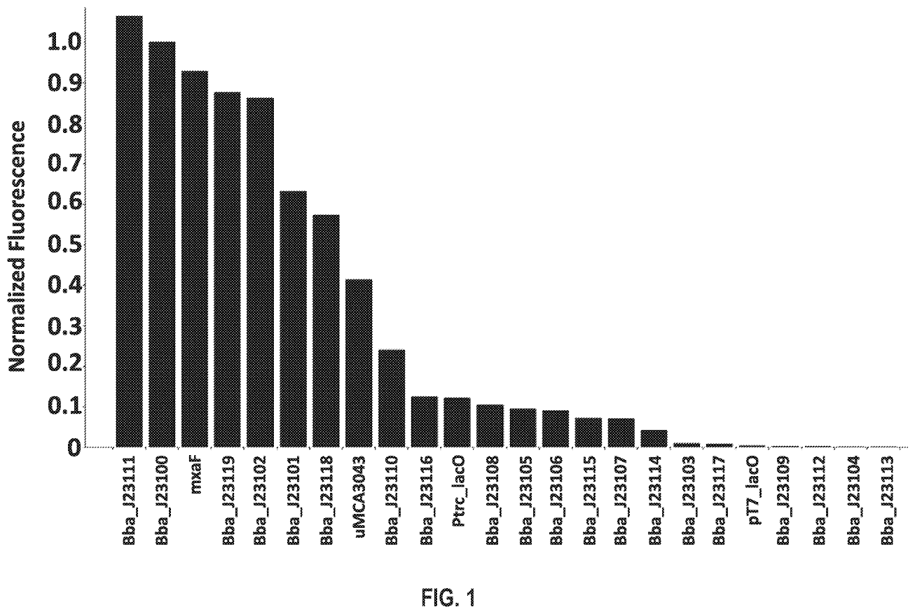

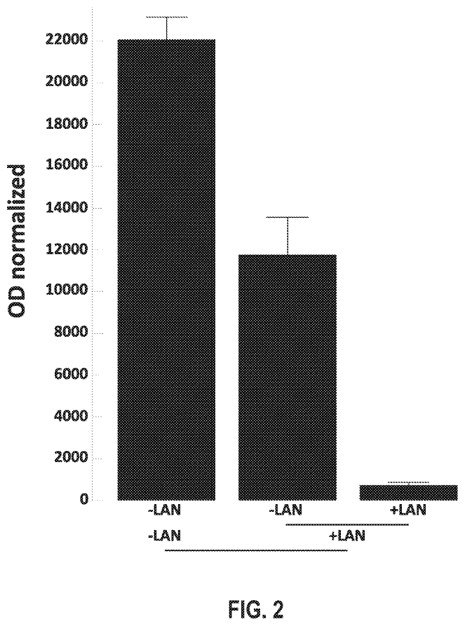

Constructs having different promoters driving the expression of mCherry were transformed into Methylococcus capsulatus. Single colonies were plasmid tested and confirmed as being mutation free. These clones were further tested for activity. See FIG. 1. The following promoters were tested for activity pMxaF, J23111, J12100, J23102, pBAD, J23110, lacO, J23116, J23106, J23105, J23108, J23107, J23115, J23114, J23118, J23104, J23101, J23119, and uMCA3034. pMxaF was chosen for further testing in the presence of lanthanum. Methylococcus capsulatus transformed with pMxaF-mCherry showed that in the presence of 35 μM lanthanum, expression is repressed by approximately 90%. See FIG. 2. After removal of the 35 μM lanthanum, approximately 50% activity is recovered after 24 hours.

### Example 2: Lanthanum Titration

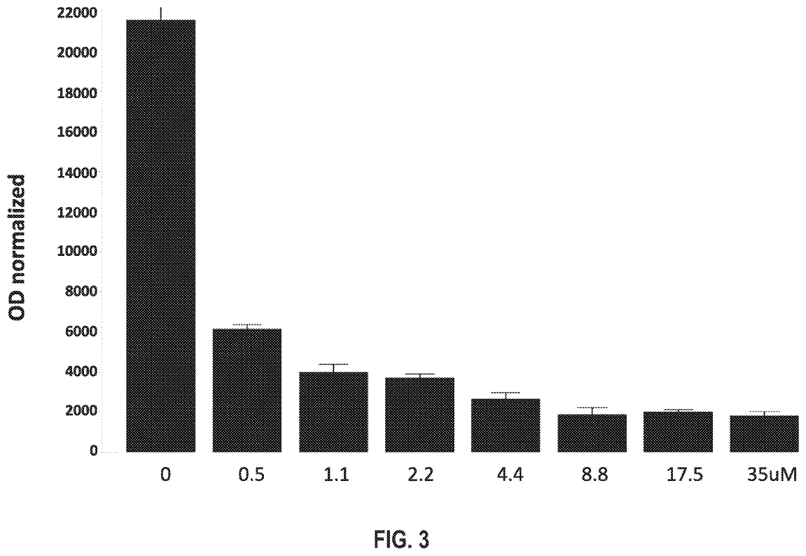

Methylococcus capsulatus transformed with pMxaF-mCherry were used to determine the optimal concentration necessary for repression of the pMxaF promoter. A Methylococcus capsulatus with a pMxaF-mCherry promoter was grown in the presence of 0, 0.5, 1.1, 2.2, 4.4, 8.8, 17.5, and 35 μM lanthanum. At 0.5 μM, pMxaF is repressed by approximately 70%. At 8.8 μM, repression is seen at approximately 87%, similar to that seen at 17.5, and 35 μM. See FIG. 3.

### Example 3: Reactivation

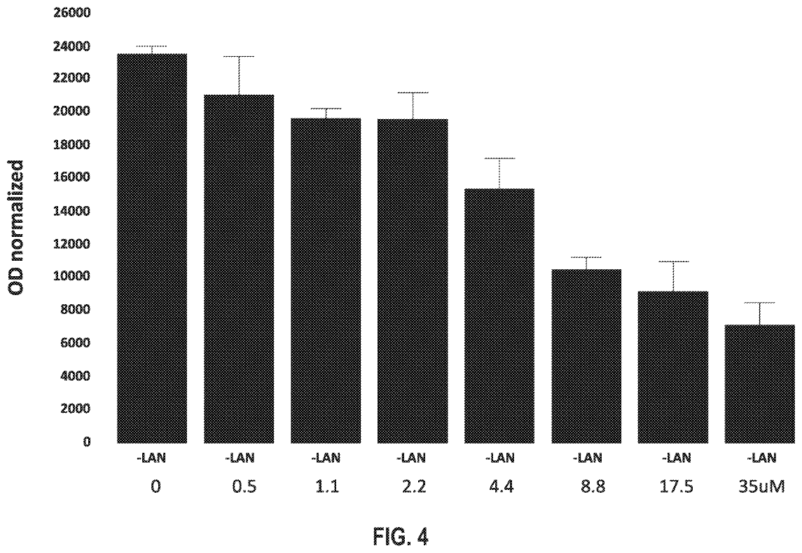

The methanotrophs from FIG. 3 were used in a “depression” experiment, where the media containing the various amount of lanthanum in FIG. 3, were diluted to the point where there is effectively no lanthanum left in the media, e.g. (a 30-fold dilution). mCherry expression was measured 24 hours after dilution. Generally, the more lanthanum used to repress pMxaF, the less expression is recovered after lanthanum is removed from the system. See FIG. 4. For example, in the samples that received 8.8 μM lanthanum initially and later depressed by dilution, mCherry expression recovered approximately 50%.

In order to ensure that the cells were sufficiently diluted and no lanthanum was present extracellularly, the methanotrophs were washed an additional three times. The amount of expression following three additional washings did not change. Thus, the lack of recovery is likely due to residual intracellular lanthanum, which could not be diluted out even after multiple washing/dilutions.

### Example 4: pBAD and Arabinose

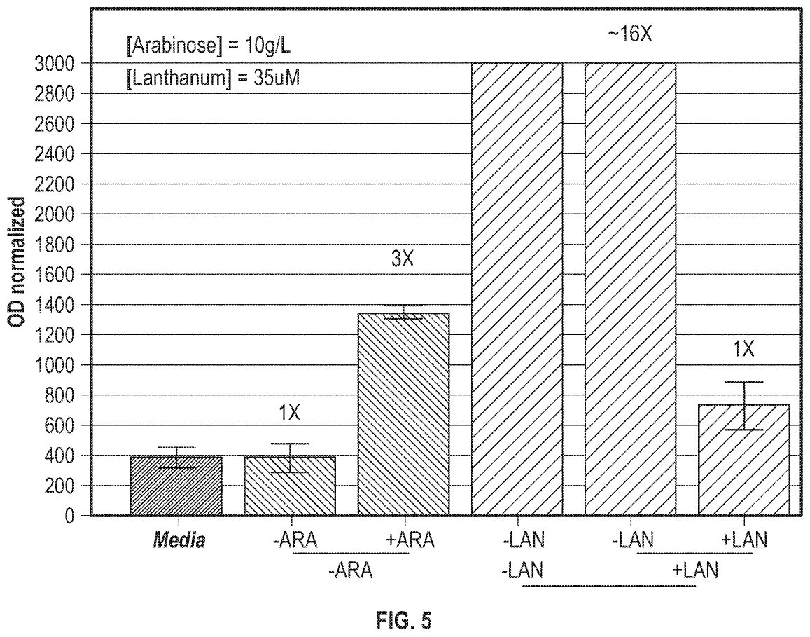

Methylococcus capsulatus transformed with pBAD driving mCherry expression were tested for the ability to be induced by other compounds such as sugars. For example, pBAD in the presence of 10 g/L of arabinose was induced by approximately 300% (i.e., 3 fold). See FIG. 5. mCherry levels without any arabinose is identical to the levels seen in the background media.

### Example 5: pXoxF and Lanthanum

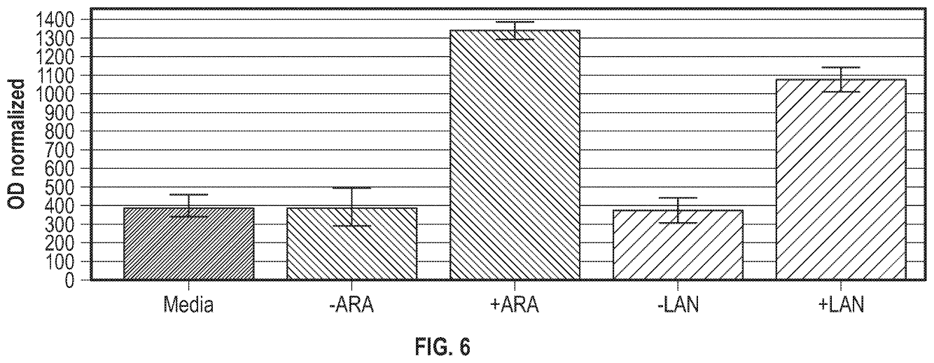

Methylococcus capsulatus transformed with pXoxF driving mCherry expression was tested for its ability to respond to lanthanum. In the presence of 35 μM lanthanum, pXoxF was activated, increasing the expression of pXoxF by approximately 300% (3 fold), similar to levels exhibited by arabinose on the pBAD promoter. See FIG. 6 (arabinose treatment was conducted on a pBAD promoter while lanthanum treatment was conducted on a pXoxF promoter). This study indicates that pXoxF has a tight “off” state, with minimal leaky expression. It also indicates that pXoxF can be induced by lanthanum.

### Example 6: Isobutanol Producing Strains

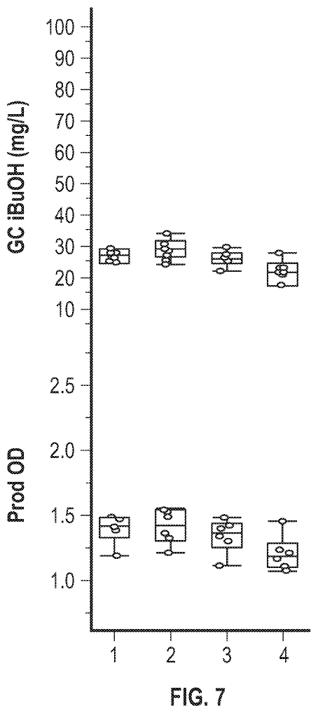

Methylococcus capsulatus strains expressing isobutanol production pathway enzymes driven by a pMxaF promoter shows the ability of using a lanthanum switch to produce isobutanol. Four different strains having isobutanol enzymes driven by pMxaF were able to produce of isobutanol after lanthanum was diluted out from the initial medium. See FIG. 7. As seen, OD levels were similar among the four different strains.

### Example 7: 2,3-Butanediol Producing Strains

In order to produce 2,3-BDO, Methylococcus capsulatus was transformed with genes that gave it the ability to produce 2,3-BDO. These genes were also placed under the control of a lanthanum “switch” where lanthanum repressed the expression of genes in its presence. Upon removal or dilution of lanthanum in the media, the repressed genes were “switched” on.

Methylococcus capsulatus expressing the various genes described below in Table 1 were pre-culturing in the presence of 10 μM lanthanum.

TABLE 1

| Number | Strain | Genotype |
|---|---|---|
| 1 & 22 | XZ685 | pmxaF>Bsu.alsA>rbs.GTW0001_Kpn.BudA>rbs.GTW0001_Cau.ButA |
| 2 & 23 | XZ686 | |
| 3 & 24 | XZ687 | |
| 4 & 25 | XZ688 | pmxaF>Bsu.alsA>rbs.Mca.mxaF_Kpn.BudA>rbs.Mca.mxaF_Cau.ButA |
| 5 & 26 | XZ689 | |
| 6 & 27 | XZ690 | |
| 7 & 28 | XZ691 | pmxaF>Blic.alsA>rbs.GTW0001_Kpn.BudA>rbs.GTW0001_Cau.ButA |
| 8 & 29 | XZ692 | |
| 9 & 30 | XZ693 | |
| 10 & 31 | XZ694 | pmxaF>Blic.alsA>rbs.Mca.mxaF_Kpn.BudA>rbs.Mca.mxaF_Cau.ButA |
| 11 & 32 | XZ695 | |
| 12 & 33 | XZ696 | |
| 13 & 34 | XZ697 | pmxaF>Bsu.alsA>rbs.GTW0001_Kpn.BudA>rbs.GTW0001_Cau.ButA |
| 14 & 35 | XZ698 | |
| 15 & 36 | XZ699 | |
| 16 & 37 | XZ700 | pmxaF>Bsu.alsA>rbs.Mca.mxaF_Kpn.BudA>rbs.Mca.mxaF_Cau.ButA |
| 17 & 38 | XZ701 | |
| 18 & 39 | XZ702 | |
| 19 & 40 | XZ703 | pmxaF>Blic.alsA>rbs.Mca.mxaF_Kpn.BudA>rbs.Mca.mxaF_Cau.ButA |
| 20 & 41 | XZ704 | |
| 21 & 42 | XZ705 | |

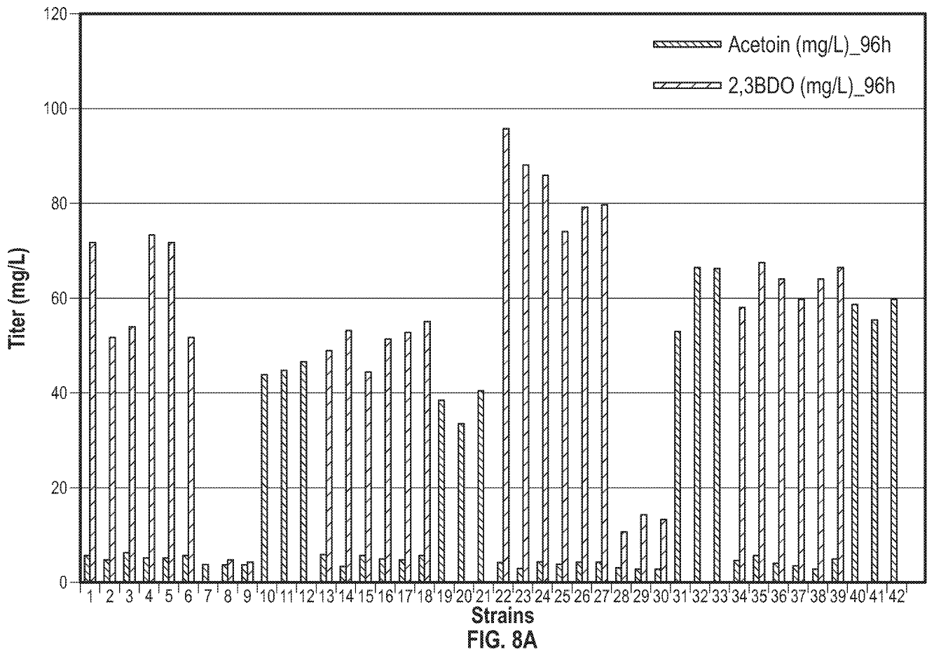

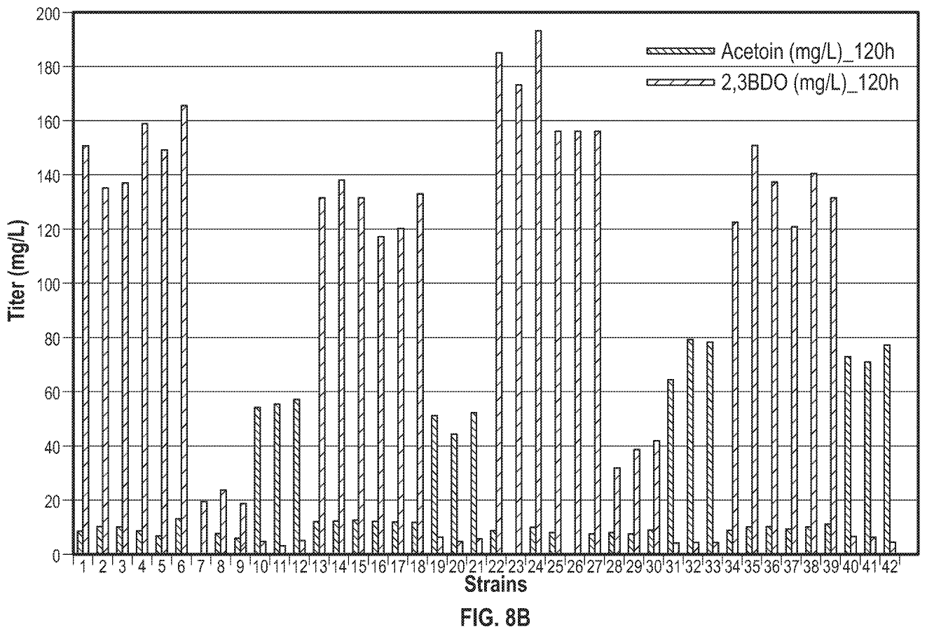

After growing precultures to ˜3 OD600, the medium containing lanthanum was diluted at a ratio of 1:10 (lanthanum containing media: non-lanthanum containing media) or 1:50. After 96 or 120 hours, the cultures assessed for production of 2,3-BDO and acetoin (FIG. 8A, 96 hours) or (FIG. 8B, 120 hours). Shown below in Table 2 are strains, dilution levels, acetoin production titers after 96 hours, 2,3-BDO production titers after 96 hours, acetoin production titers after 120 hours, and 2,3-BDO production titers after 120 hours.

TABLE 2

| | | | Acetoin | 2,3BDO | Acetoin | 2,3BDO |
|---|---|---|---|---|---|---|
| | Strain | Dilution | (mg/L)_96 h | (mg/L)_96 h | (mg/L)_120 h | (mg/L)_120 h |
| 1 | XZ685 | 10X | 5.847 | 71.342 | 8.617 | 150.864 |
| 2 | XZ686 | 10X | 4.86 | 51.439 | 10.539 | 135.871 |
| 3 | XZ687 | 10X | 6.306 | 53.591 | 10.475 | 137.104 |
| 4 | XZ688 | 10X | 5.311 | 73.08 | 8.79 | 158.947 |
| 5 | XZ689 | 10X | 5.282 | 71.059 | 6.944 | 149.219 |
| 6 | XZ690 | 10X | 5.679 | 51.291 | 13.412 | 165.688 |
| 7 | XZ691 | 10X | 4.014 | 0 | 0 | 19.231 |
| 8 | XZ692 | 10X | 3.719 | 4.679 | 7.984 | 23.991 |
| 9 | XZ693 | 10X | 3.778 | 4.082 | 6.2 | 18.78 |
| 10 | XZ694 | 10X | 43.925 | 0 | 54.582 | 4.871 |
| 11 | XZ695 | 10X | 44.734 | 0 | 55.632 | 3.363 |
| 12 | XZ696 | 10X | 46.473 | 0 | 57.444 | 5.39 |
| 13 | XZ697 | 10X | 5.982 | 48.549 | 12.221 | 131.618 |
| 14 | XZ698 | 10X | 3.441 | 52.775 | 12.594 | 137.996 |
| 15 | XZ699 | 10X | 5.747 | 44.14 | 12.937 | 131.699 |
| 16 | XZ700 | 10X | 5.101 | 50.998 | 12.26 | 117.653 |
| 17 | XZ701 | 10X | 4.82 | 52.523 | 12.167 | 120.369 |
| 18 | XZ702 | 10X | 5.802 | 54.763 | 12.119 | 133.294 |
| 19 | XZ703 | 10X | 38.501 | 0 | 51.296 | 6.482 |
| 20 | XZ704 | 10X | 33.64 | 0 | 44.195 | 5.021 |
| 21 | XZ705 | 10X | 40.326 | 0 | 52.443 | 5.978 |
| 22 | XZ685 | 50X | 4.245 | 95.288 | 8.993 | 184.974 |
| 23 | XZ686 | 50X | 3.028 | 87.876 | 0 | 173.315 |
| 24 | XZ687 | 50X | 4.341 | 85.562 | 10.06 | 193.196 |
| 25 | XZ688 | 50X | 4.088 | 73.804 | 8.181 | 156.027 |
| 26 | XZ689 | 50X | 4.273 | 78.782 | 0 | 156.435 |
| 27 | XZ690 | 50X | 4.383 | 79.226 | 7.879 | 155.869 |
| 28 | XZ691 | 50X | 3.178 | 10.35 | 8.197 | 32.164 |
| 29 | XZ692 | 50X | 2.838 | 14.16 | 7.839 | 38.733 |
| 30 | XZ693 | 50X | 2.865 | 12.942 | 9.163 | 41.967 |
| 31 | XZ694 | 50X | 53.138 | 0 | 64.47 | 4.259 |
| 32 | XZ695 | 50X | 66.256 | 0 | 79.665 | 4.736 |
| 33 | XZ696 | 50X | 66.139 | 0 | 78.46 | 4.746 |
| 34 | XZ697 | 50X | 4.664 | 57.939 | 9.411 | 122.655 |
| 35 | XZ698 | 50X | 5.749 | 67.055 | 10.424 | 151.002 |
| 36 | XZ699 | 50X | 4.182 | 63.379 | 10.386 | 137.577 |
| 37 | XZ700 | 50X | 3.558 | 59.187 | 9.519 | 121.163 |
| 38 | XZ701 | 50X | 3.085 | 63.574 | 10.265 | 140.806 |
| 39 | XZ702 | 50X | 5.039 | 66.163 | 11.252 | 131.931 |
| 40 | XZ703 | 50X | 58.687 | 0 | 72.718 | 6.653 |
| 41 | XZ704 | 50X | 55.523 | 0 | 70.855 | 6.577 |
| 42 | XZ705 | 50X | 59.577 | 0 | 76.618 | 4.793 |

As shown FIGS. 8A, 8B, and Table 2, strains XZ685, XZ686, XZ687, XZ688, XZ689, and XZ690 (referring to 22 to 27, respectively in FIGS. 8A and 8B) produced the highest titers of 2,3-BDO at both 96 and 120 hours when diluting the lanthanum containing medium 1:50 (50×). Using a 1:10 dilution protocol also resulted in significant production of 2,3-BDO but the titers were lower than those using the 1:50 dilution protocol at both 96 and 120 hours. Strains XZ697, XZ698, XZ699, XZ700, XZ701, and XZ702 (referring to 34 to 39, respectively in FIGS. 8A and 8B) produced lower titers at both 1:10 and 1:50 dilutions at both 96 and 120 hours.

### Example 8: Other Rare Earth Metals

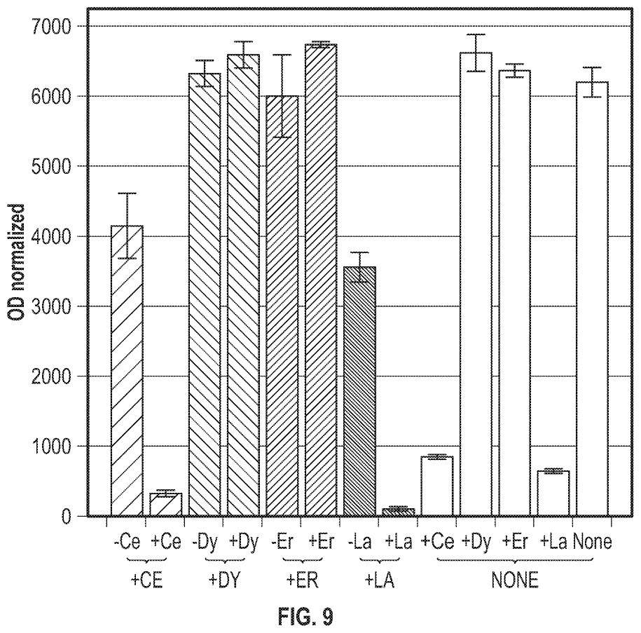

In order to determine whether other rare earth metals can be used to modify the expression of genes, Cerium (Ce), dysprosium (Dy), and erbium (Er) were placed into the media in the presence of a Methylococcus capsulatus having a pMxaF promoter driving mCherry expression. FIG. 9 shows the ability of other rare earth metals to activate or repress the pMxaF promoter as measured by mCherry levels. Cerium (Ce) and Lanthanum (La) were able to repress the pMxaF promoter whether or not they were present in the media during pre-culture or added after the pre-culture period. Further, dysprosium (Dy) or erbium (Er) were not able to repress the pMxaF promoter at any time. See FIG. 9.

Also as shown in FIG. 9, repression of the promoter by lanthanum or cerium were more effective if initially present. Addition of lanthanum or cerium at a later time lead to decreased expression of mCherry, however the repression was not as effective. The addition of dysprosium or erbium at either time point did not lead to activation of repression of the pMxaF promoter.

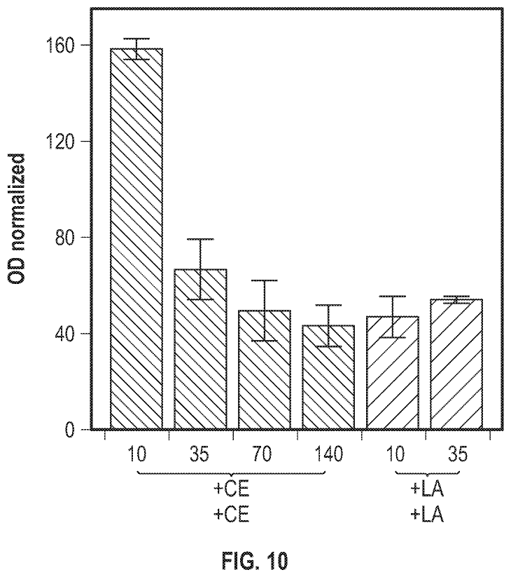

A titration study was also performed using 10, 35, 70, and 140 μM cerium. As shown in FIG. 10, full repression of the pMxaF promoter occur at approximately 70 μM of cerium. At this level, repression is similar to that of 10 or 35 μM of lanthanum.

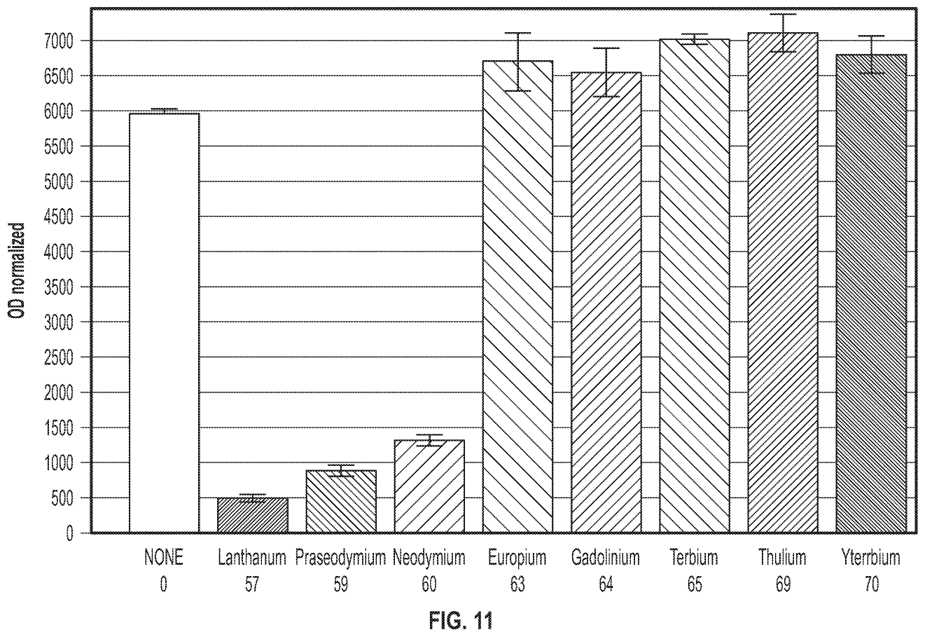

Other rare earth metals were also tested. As seen in FIG. 11, lanthanum (La), praseodymium (Pr), and neodymium (Nd), repressed the pMxaF promoter. However, europium (Eu), gadolinium (Gd), terbium (Tb), thulium (Tm), and ytterbium (Yb), did not repress the pMxaF promoter, but rather activated the expression of the pMxaF promoter.

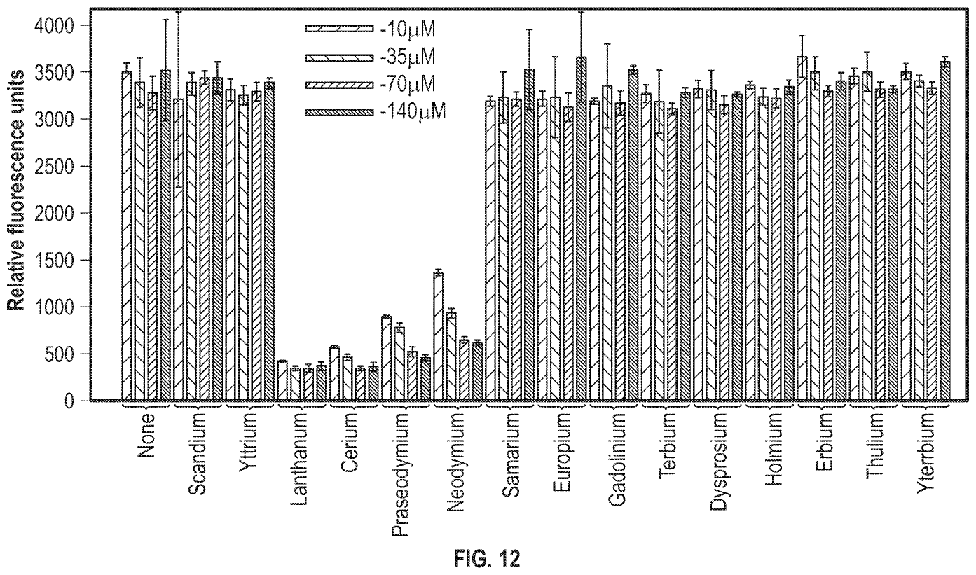

An additional experiment was conducted using scandium (Sc), yttrium (Y), lanthanum (La), cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), and ytterbium (Yb). Four concentrations of these rare earth metals (10 μM, 35 μM, 70 μM, or 140 μM) were tested for its ability to activate or repress the pMxaF promoter as measured by mCherry. The cultures were treated with for 24 hours with the respective rare earth metal. As seen in FIG. 12, scandium (Sc), yttrium (Y), samarium (Sm), europium (Eu), gadolinium (Gd), terbium (Tb), dysprosium (Dy), holmium (Ho), erbium (Er), thulium (Tm), and ytterbium (Yb) minimally activated or repressed the pMxaF promoter at 10 μM, 35 μM, 70 μM, or 140 μM. On the other hand, lanthanum (La), cerium (Ce), praseodymium (Pr), and neodymium (Nd) significantly repressed the expression of the pMxaF promoter at all concentrations. As previously shown and verified here, lanthanum repressed the pMxaF promoter and maximal repression was observed at 35 μM. Additionally, cerium repressed the pMxaF promoter and maximal repression was observed at 70 μM. Further, praseodymium and neodymium repressed the pMxaF promoter and maximal repression was observed at 140 μM.

1-69. (canceled)

70. A vector comprising:
(a) genes encoding the following polypeptides:
(i) an acetolactate synthase comprising an amino acid sequence that is at least 90% identical to SEQ ID NO: 5;
(ii) an alpha-acetolactate decarboxylase comprising an amino acid sequence that is at least 90% identical to SEQ ID NO: 9; and
(iii) an acetoin reductase comprising an amino acid sequence that is at least 90% identical to SEO ID NO: 11; and
(b) a promoter driving the expression of such genes, the promoter being responsive to a rare earth metal and is-active in a methanotroph.

71. The vector of claim 70, wherein the rare earth metal is cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), scandium (Sc), terbium (Tb), thulium (Tm), ytterbium (Yb), or yttrium (Y).

72. The vector of claim 70, wherein the rare earth metal is cerium.

73. The vector of claim 72, wherein the promoter is responsive to cerium at a concentration of at least 10 μM.

74. The vector of claim 70, wherein the rare earth metal is lanthanum.

75. The vector of claim 74, wherein the promoter is responsive to lanthanum at a concentration of at least 0.5 μM.

76-84. (canceled)

85. A method of making a genetically modified microorganism capable of converting a C1 carbon to a multicarbon product comprising transforming a microorganism with the vector of claim 70.

86. (canceled)

87. A method of making a multicarbon product from a C1 carbon, the method comprising:
(a) contacting a microorganism with the vector of claim 70, thereby generating a genetically-modified microorganism;
(b) contacting the genetically-modified microorganism with a C1 carbon; and
(c) growing the methanotroph to produce the multicarbon product.

88. The method of claim 87, wherein the multicarbon product is 2,3-butanediol.

89-91. (canceled)

92. The method of claim 87, further comprising contacting the multicarbon product with a catalyst to produce a different product.

93. (canceled)

94. The method of claim 92, wherein the different product is butadiene.

95. The method of claim 92, wherein the catalyst is a SiO₂-supported cesium dihydrogen phosphate (CsH₂PO₄) catalyst.

96. The method of claim 92, wherein the different product is methyl ethyl ketone.

97-102. (canceled)

103. The method of claim 87, further comprising contacting the microorganism with media having a rare earth metal that can activate or repress a molecular switch.

104-106. (canceled)

107. The method of claim 103, wherein the rare earth metal is lanthanum (La).

108. The method of claim 107, wherein the lanthanum (La) is present at a concentration of at least 0.5 μM.

109-127. (canceled)

128. The vector of claim 70, wherein the promoter is pMxAF or pXoxF.

129. The vector of claim 70, wherein the genes encode the following polypeptides:
an acetolactate synthase comprising an amino acid sequence that is at least 95% identical to SEQ ID NO: 5;
an alpha-acetolactate decarboxylase comprising an amino acid sequence that is at least 95% identical to SEQ ID NO: 9; and
an acetoin reductase comprising an amino acid sequence that is at least 95% identical to SEQ ID NO: 11.

130. The vector of claim 70, wherein:
(a) the acetolactate synthase comprises an amino acid sequence that is at least 98% identical to SEQ ID NO: 5;
(b) the alpha-acetolactate decarboxylase comprises an amino acid sequence that is at least 98% identical to SEQ ID NO: 9; and
(c) the acetoin reductase comprises an amino acid sequence that is at least 98% identical to SEQ ID NO: 11.

131. The vector of claim 70, wherein:
(a) the acetolactate synthase comprises the amino acid sequence of SEQ ID NO: 5;
(b) the alpha-acetolactate decarboxylase comprises the amino acid sequence of SEQ ID NO: 9; and
(c) the acetoin reductase comprises the amino acid sequence of SEQ ID NO: 11.


SEQUENCE LISTING (82 sequences)

SEQ ID NO: 1 - protein, 570 aa, Bacillus subtilis
MTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 2 - DNA, 1713 bp, Bacillus subtilis
  source 1..1713: mol_type = genomic DNA
atgaccaaggccaccaaggaacagaaaagcctggtcaagaaccgcggtgctgaactggttgtggactgcctcgtggaacagggcgtgacccatgtcttcggcatcccgggcgccaagatcgacgccgtcttcgacgccctgcaggataaaggtccggaaatcatcgtggcacgccatgagcagaacgcagccttcatggcccaggccgtcggtcggctgacgggtaagcccggcgtggtgctggtcacctccggtccgggagcctcgaacctggccacgggactgctcaccgccaacaccgaaggcgacccggtggtcgccctggccggtaatgtcatccgggcggatcgcctgaagcgcacccatcagtccctggataacgcggccctgttccagccaatcaccaaatatagtgtcgaagtgcaggatgtgaagaacatcccggaagccgtcaccaatgcgttccgaatcgcgtccgccggccaagcaggggcagcattcgtgagcttcccccaggacgtggtcaatgaagtgaccaacaccaaaaacgtcagagccgtagccgccccgaagctgggccctgcagcagatgacgccatctccgctgccatcgcgaagatccagaccgcaaagctgccggtcgtgctggtcggaatgaagggcggacgcccggaggccatcaaggccgtgcgtaaactgctgaagaaggtgcagctaccgttcgtggaaacctaccaggccgccggcaccctgagtcgggacttggaagaccagtatttcggccgtatcggcctgttccgcaaccagccgggcgacctgctcctggaacaagccgatgtggtgctgaccatcggctacgacccgatcgaatatgacccgaagttctggaacatcaatggcgaccgcacgatcatccatctggacgaaatcatcgccgacatcgaccatgcctatcagccggacctggaactgatcggcgacatcccgagcaccatcaaccacatcgaacacgatgccgtgaaggtggaatttgccgaacgcgaacagaagatcctgtcggacctgaagcagtatatgcatgagggcgaacaggtgcctgccgactggaagtcggacagagcccatccgctggaaatcgtgaaggaactgcgtaacgccgtcgacgaccatgtcaccgtcacctgcgatatcggcagccatgccatttggatgagccgctacttccggagctatgaaccgctgaccctgatgatctccaacggtatgcagaccctcggcgtcgccctcccgtgggccatcggcgcaagtctggtgaagccgggcgaaaaagtggtcagcgtgtccggcgacggcggcttcctgttctccgctatggaactggaaaccgcggtccgcctgaaggccccgatcgtgcatatcgtgtggaacgacagcacctacgacatggtcgccttccagcagctgaaaaagtacaaccgcaccagcgccgtggacttcggcaatatcgacatcgtgaagtatgccgaatccttcggagccaccggactgcgcgtggaatccccggaccagctggcggacgttctgcgtcagggcatgaatgccgaaggtcccgtgattatcgatgtgcccgtcgactacagcgacaacatcaacctggcctcggacaaattgccgaaggagttcggcgaactgatgaaaacaaaagcactataa

SEQ ID NO: 3 - protein, 556 aa, Clostridium autoethanogenum
MNRDIKKEVQLNTAQMLVKCLEAEGVKYIFGIPGEENLEIMNAISDSTIEFITTRHEQGAAFMADVYGRLTGKAGVCLSTLGPGATNLVTGVADADSDGAPVVAITGQVGTERMHITSHQFLDLCKMFEPITKRSKQIVRPDTVSEIIRLVFKYAESEKPGACHIDLPVNIAKMPVGALEKPLEKKIPPKEHADLSTIEEAASEIFKAKNPIILAGSGAIRGNSSKAVTEFATKLKIPVINTMMAKGIIPMDNKYSMWTIGIPQKDYVNKIIEEADLVITIGYDIVEYAPSKWNINGDIKIVHIDARPSHINKLYQPIVEVVGDISDALYNILRRTSSKDEPVKALEIKSEMLAEHESYANDNAFPMKPQRILNDVRKVMGPHDIVISDVGAHKMWIARHYNCYEPNTCIISNGFATMGIGVPGAIAAKLINPDKKVLAIVGDGGFMMNNQELETALRIKTPIVVLIFNDSNYGLIKWKQEEHYGKSCYVDFTNPDFVKLAESMYAKGYRVEKAEDLIPTLEEAFKQNVPAVIDCQVDYGENIKLTKHLKEVYENM

SEQ ID NO: 4 - DNA, 1671 bp, Clostridium autoethanogenum
  source 1..1671: mol_type = genomic DNA
atgaatcgggatatcaagaaagaggtgcagctcaacacggcccagatgctggtcaagtgtctggaagccgagggcgtcaagtatatcttcggcatcccgggcgaggagaatctcgaaatcatgaacgccatctcggattccacgatcgagttcatcaccacccgccatgaacagggcgcggccttcatggccgacgtgtacggccggctgaccggcaaggcgggcgtgtgtctgagcaccctcggccccggcgcgaccaacctggtcaccggcgtggccgacgccgactccgacggcgcccccgtggtcgcgatcaccggccaggtgggcacggagcggatgcacatcacctcccatcagttcctcgacctctgcaagatgttcgagccgatcaccaagcggagcaagcagatcgtccgcccggacacggtgtcggagatcatccgcctggtgttcaagtacgccgaaagcgaaaagcccggcgcctgtcatatcgacctgccggtcaacatcgccaagatgcccgtcggcgccctggagaagccgctggagaaaaaaatcccgccgaaggaacacgcggacctgtccaccatcgaggaagcggcgtccgagatcttcaaggccaaaaaccccatcatcctggccggcagcggcgccatccgcggcaacagcagcaaggcggtcaccgagttcgccaccaagctgaagatccccgtcatcaacacgatgatggccaagggcatcatcccgatggacaacaagtatagcatgtggaccatcggcatcccccagaaggactatgtgaacaagatcatcgaagaggccgacctggtcatcaccatcggctacgacatcgtggaatatgccccgtcgaaatggaacatcaacggcgacatcaagatcgtccatatcgacgcccgcccctcgcacatcaacaaactctaccagcccatcgtggaggtggtcggcgacatcagcgacgcgctgtataacatcctgcgccgcaccagctcgaaagacgagccggtcaaggcgctggagatcaagtcggaaatgctggcggagcacgagtcctacgcgaacgacaatgcgttcccgatgaagccgcagcgcatcctcaacgatgtgcgcaaagtcatgggcccgcacgacatcgtgatctccgatgtgggcgcccataaaatgtggatcgcccgccactataactgctacgagccgaatacctgcatcatctcgaacggcttcgccacgatgggcatcggcgtcccgggcgcgatcgccgccaaactcatcaacccggataagaaggtcctggccatcgtcggcgacggcggcttcatgatgaataaccaggaactggagacggcgctgcgcatcaaaacgcccatcgtggtcctcatcttcaacgactccaattacggcctcatcaagtggaagcaggaggagcattatggcaaatcgtgctatgtggacttcaccaacccggacttcgtgaagctggccgagagcatgtacgccaaaggctatcgcgtggagaaagccgaggatctgatcccgaccctcgaagaggccttcaagcagaatgtcccggcggtcatcgactgccaggtggactatggcgagaatatcaagctcaccaagcacctcaaggaggtctatgaaaacatgtga

SEQ ID NO: 5 - protein, 572 aa, Bacillus licheniformis
MNNVAAKNETLTVRGAELVVDSLIQQGVTHVFGIPGAKIDAVFDVLKDKGPELIVCRHEQNAAFMAAAVGRLTGKPGVCLVTSGPGASNLATGLVTANTEGDPVVALAGAVKRADRLKKTHQSMDNAALFQPITKYSAEVEDANNIPEAVTNAFRAAASGQAGAAFLSFPQDVTAGPATAKPVKTMPAPKLGAASDEQISAAIAKIHNANLPVVLVGMKGGRPEAIEAVRRLLRKVKLPFVETYQAAGTLSHDLEDQYFGRIGLFRNQPGDMLLEKADVVLTVGYDPIEYDPVFWNGKGERSVIHLDEIQADIDHDYQPEIELIGDIAETLNHIEHDSLPVSIDESFAPVLDYLKKALEEQSEPPKETKTDLVHPLQIVRDLRELLSDDITVTCDIGSHAIWMSRYFRTYRPHGLLISNGMQTLGVALPWAIAATLVNPGQKVVSVSGDGGFLFSAMELETAVRLKAPIVHIVWNDSTYDMVAFQQEMKYKRTSGVDFGGIDIVKYAESFGAKGLRVNSPDELAEVLKAGLDAEGPVVIDIPVDYSDNIHLADQRFPKKFEEHFNKEASKQS

SEQ ID NO: 6 - DNA, 1719 bp, Bacillus licheniformis
  source 1..1719: mol_type = genomic DNA
atgaataacgtcgcggccaagaacgaaaccctgaccgtccggggcgccgaactcgtggtggatagcctgatccagcagggcgtgacccatgtcttcggcatcccgggcgccaaaatcgacgcggtcttcgacgtgctgaaggataagggccccgaactgatcgtctgccgccatgagcagaacgcggccttcatggccgccgccgtcggccgcctgacgggcaagccgggcgtctgcctggtcacctccggcccgggcgcctcgaatctcgcgaccggcctggtcaccgcgaacacggaaggcgacccggtggtcgccctggcgggcgccgtgaagcgggcggatcggctgaagaagacgcaccagtcgatggataacgccgccctgttccagcccatcacgaagtacagcgcggaggtggaagacgcgaacaacatcccggaggccgtgacgaacgccttccgcgccgcggcgtccggccaggccggcgcggccttcctcagcttcccccaggatgtcaccgccggcccggccaccgccaagccggtcaaaaccatgcccgccccgaagctgggcgccgcgagcgatgaacagatctccgccgcgatcgcgaagatccacaacgcgaatctgccggtggtcctcgtgggcatgaagggcggccggccggaagccatcgaagccgtgcgccgcctgctccgcaaggtcaagctcccgttcgtggaaacctaccaggcggccggcacgctgtcgcacgatctggaggatcagtacttcggccggatcggcctgttccggaaccagccgggcgacatgctcctggaaaaggccgacgtggtcctgaccgtgggctacgacccgatcgagtacgatccggtgttctggaatggcaaaggcgaacgctcggtcatccacctcgacgaaatccaggccgatatcgatcacgactaccagcccgagatcgaactcatcggcgacatcgcggaaaccctcaatcacatcgagcatgactcgctgccggtgtccatcgacgaatccttcgcgcccgtgctcgactatctcaagaaggcgctcgaagaacagtcggagcccccgaaggaaacgaagaccgatctggtccacccgctccagatcgtgcgcgacctgcgcgagctgctctccgatgacatcaccgtcacctgcgacatcggcagccacgccatctggatgtcccgctatttccgcacctatcgcccgcatggcctcctgatctccaacggcatgcagacgctgggcgtcgccctgccgtgggcgatcgccgcgaccctggtgaacccgggccagaaggtggtgtcggtcagcggcgatggcggcttcctcttctccgcgatggaactcgaaaccgccgtccgcctcaaggcgccgatcgtgcacatcgtgtggaacgactccacgtacgacatggtcgcgttccagcaggaaatgaagtacaagcgcacctccggcgtcgatttcggcggcatcgacatcgtcaagtatgcggaatccttcggcgccaaaggcctccgcgtgaatagccccgatgaactggccgaggtcctgaaggccggcctcgacgcggagggcccggtggtcatcgacatccccgtcgactactcggataacatccacctggccgaccagcgcttcccgaagaagttcgaggagcacttcaacaaggaagcgtcgaagcagtcctga

SEQ ID NO: 7 - protein, 239 aa, Clostridium autoethanogenum
MDDEVKVPNHIYQMSTINALVSGLYDGCVSLSKLLKKGNFGIGTFKGLDGELTLLNGTFYRTKPDGSVYVCSKNVSVPFAVVTELENYNTYNIQNRTSYEDIRKELDSFIESKNIFYAFYMEGKFNYVKTRTVVKQNMPYKPMAEVVKDQPMFEYNGVDGYVVGFRCPDYVEGLNVPGYHFHFINKDKKFGGHISEFSIENAKVYVQNCSCFRMELPKNESFYNMEVQDRNDEITSVEK

SEQ ID NO: 8 - DNA, 720 bp, Clostridium autoethanogenum
  source 1..720: mol_type = genomic DNA
atggatgatgaggtgaaagtcccgaaccacatctaccagatgtcgaccatcaatgccctggtcagcggcctctacgacggctgtgtgtcgctctcgaagctcctgaaaaagggcaatttcggcatcggcacgttcaagggcctggatggcgagctgaccctcctgaacggcacgttctatcgcaccaaaccggatggctccgtgtacgtgtgcagcaagaacgtgagcgtccccttcgcggtcgtcaccgagctggagaactacaatacctataacatccagaatcgcacctcctatgaggacatccgcaaggagctggactcgttcatcgagtcgaagaacatcttctatgccttctatatggaaggcaaattcaactacgtcaaaacccgcaccgtcgtgaagcagaacatgccgtacaagccgatggccgaggtggtcaaagaccagccgatgttcgaatacaacggcgtcgatggctacgtcgtcggcttccggtgcccggattatgtggaaggcctcaatgtgcccggctaccatttccacttcatcaacaaggacaaaaagttcggcggccacatctccgagttctcgatcgagaacgccaaagtctacgtccagaactgctcctgtttccgcatggagctcccgaagaatgagagcttctacaacatggaggtccaggaccgcaacgacgaaatcacgtccgtggagaaatga

SEQ ID NO: 9 - protein, 259 aa, Klebsiella pneumoniae
MNHSAECTCEESLCETLRAFSAQHPESVLYQTSLMSALLSGVYEGSTTIADLLKHGDFGLGTFNELDGELIAFSSQVYQLRADGSARKAQPEQKTPFAVMTWFQPQYRKTFDHPVSRQQLHEVIDQQIPSDNLFCALRIDGHFRHAHTRTVPRQTPPYRAMTDVLDDQPVFRFNQREGVLVGFRTPQHMQGINVAGYHEHFITDDRKGGGHLLDYQLDHGVLTFGEIHKLMIDLPADSAFLQANLHPDNLDAAIRSVES

SEQ ID NO: 10 - DNA, 780 bp, Klebsiella pneumoniae
  source 1..780: mol_type = genomic DNA
atgaaccactcggccgaatgcacctgcgaagagagcctctgcgaaaccctccgggccttctcggcccagcacccggagagcgtcctgtaccagacgagcctgatgtcggcgctgctgtcgggcgtgtatgaaggctcgacgaccatcgccgacctgctgaagcatggcgacttcggcctgggcaccttcaatgaactggacggcgagctcatcgccttcagctcgcaggtgtatcagctccgggccgatggctccgcccggaaggcccagcccgaacagaagaccccgttcgccgtgatgacctggttccagccgcagtatcggaagaccttcgaccaccccgtgagccgccagcagctccacgaggtgatcgaccagcagatcccgagcgacaacctcttctgcgccctgcgcatcgacggccatttccgccacgcgcatacccgcaccgtcccgcggcagaccccgccctaccgcgccatgaccgatgtcctggatgaccagccggtcttccggttcaaccagcgcgagggcgtcctggtcggcttccgcaccccgcagcacatgcagggcatcaacgtcgcgggctatcatgaacacttcatcaccgatgatcgcaagggcggcggccacctcctcgactaccagctggaccacggcgtcctgaccttcggcgaaatccataagctgatgatcgacctccccgccgacagcgccttcctgcaggcgaatctgcatccggacaacctcgatgccgccatccgctccgtcgagtcgtga

SEQ ID NO: 11 - protein, 357 aa, Clostridium autoethanogenum
MKAVLWYDKKDVRVEEIEEPKVKENAVKIKVKWCGICGSDLHEYLGGPIFIPVGTPHPLSKSTAPVVLGHEFSGEVVEIGSKVTKFKAGDRVIVEPIVACGKCPACLEGKYNLCEALGFHGLCGSGGGFAEYTVFPEDFVHKIPDTMDYEQAALVEPMAVALHSLRVGNFTTGNTALVLGAGPIGLATIQCLKASGARIVIVFQRKSVRQEYAKKFGADVVLDPNEVDVIEEIKKLTGGVGVDTSFETTGANVGINTAIQALKYEGTAVITSVWEKNAEINPNDLVFTEKKVVGTLAYRHEFPSTIALMNDGRIKTDGYITKRIALEDIVKEGFETLTGPEKKKHVKIIVTPDKSLL

SEQ ID NO: 12 - DNA, 1074 bp, Clostridium autoethanogenum
  source 1..1074: mol_type = genomic DNA
atgaaggccgtcctgtggtacgacaaaaaggatgtccgcgtggaagaaatcgaggaaccgaaggtgaaagaaaacgccgtgaagatcaaagtcaagtggtgcggcatctgcggctcggacctgcatgagtatctcggcggcccgatcttcatcccggtcggcaccccccacccgctgtcgaagagcaccgcgcccgtcgtgctgggccacgagttctcgggcgaagtggtggagatcggcagcaaagtgaccaagttcaaggcgggcgaccgcgtcatcgtggaaccgatcgtcgcctgcggcaaatgcccggcctgcctggaaggcaagtacaatctgtgcgaggcgctgggcttccacggcctgtgcggcagcggcggcggcttcgccgagtacacggtgttcccggaagatttcgtgcacaagatccccgacacgatggattatgaacaggccgcgctggtggagccgatggcggtcgcgctgcactccctgcgggtgggcaacttcaccacgggcaacaccgccctggtcctgggcgcgggcccgatcggcctggccaccatccagtgcctcaaagcgtcgggcgcccggatcgtcatcgtcttccagcgcaaatcggtgcggcaggaatacgccaagaagttcggcgcggacgtggtcctcgacccgaatgaggtggacgtgatcgaggaaatcaaaaagctgaccggcggcgtgggcgtggacacgagcttcgaaaccaccggcgccaacgtcggcatcaacaccgcgatccaggcgctgaaatatgagggcaccgccgtcatcacctccgtctgggagaagaacgccgagatcaatccgaacgacctggtcttcaccgaaaagaaggtcgtcggcaccctcgcgtaccggcacgagttcccgtcgaccatcgccctgatgaacgacggccgcatcaagaccgatggctatatcaccaagcggatcgccctggaagacatcgtcaaggagggcttcgaaaccctgaccggcccggagaagaaaaagcacgtcaaaatcatcgtcacgcccgataaaagcctcctgtga

SEQ ID NO: 13 - protein, 400 aa, Bacillus subtilis
MKALLWHNQRDVRVEEVPEPAVRSGAVKIKVKWCGICGTDLHEYLAGPIFIPTEEHPLTHVKAPVILGHEFSGEVVEIGEGVTNHKVGDRVVVEPIYSCGKCEACKHGHYNVCEQLVFHGLGGDGGGFSEYTVVPADMVHHIPDEMTYEQGALVEPAAVAVHAVRQSKLKEGEAVAVFGCGPIGLLVIQAAKAAGATPVIAVELSKERQELAKLAGADYVLNPAEQDVVAEIRNLTNGLGVNVSFEVTGVEVVLRQAIESTSFEGQTVIVSVWEKDATITPNNLVLKEKEVVGILGYRHIFPSVIKLISSGQIQAEKLITKKITVDQVVEEGFEALVKDKKQVKILVSPKGHLLDYQLDHGVLTFGEIHKLMIDLPADSAFLQANLHPDNLDAAIRSVES

SEQ ID NO: 14 - DNA, 1053 bp, Bacillus subtilis
  source 1..1053: mol_type = genomic DNA
atgaaagccctgctgtggcataaccagcgcgacgtgcgggtggaagaggtcccggagcccgccgtccgcagcggcgcggtgaaaatcaaagtgaaatggtgcggcatctgtggcaccgacctgcatgaatatctggccggccccatcttcatcccgacggaggaacatccgctgacgcacgtcaaggccccggtcatcctcggccatgagttcagcggcgaggtggtggagatcggcgaaggcgtcaccaatcacaaagtcggcgatcgcgtggtcgtcgaaccgatctactcgtgcggcaagtgtgaggcgtgcaagcacggccactataatgtctgcgagcagctggtgttccacggcctgggcggcgacggcggcggcttctcggagtacaccgtggtgccggcggatatggtccaccacatcccggatgaaatgacctacgagcagggcgccctggtcgagccggccgccgtggcggtgcacgcggtgcgccagagcaaactcaaggagggcgaagccgtggccgtcttcggctgcggcccgatcggcctgctggtcatccaggcggccaaagcggcgggcgcgacccccgtcatcgcggtcgagctgtcgaaggaacgccaggagctcgccaagctggcgggcgcggattatgtcctgaaccccgccgaacaggacgtggtggcggaaatccggaacctgaccaacggcctgggcgtcaacgtctccttcgaggtcaccggcgtggaagtcgtcctgcggcaggcgatcgaatcgacctcgttcgagggccagacggtcatcgtgtcggtctgggagaaggacgccaccatcacgcccaataatctggtcctgaaagagaaggaagtggtcggcatcctcggctaccggcatatcttcccgtccgtcatcaagctgatctcgtcgggccagatccaggccgagaaactcatcaccaagaagatcacggtggaccaggtggtcgaagaaggcttcgaagcgctggtcaaggataagaagcaggtgaagatcctcgtgtcgccgaagtga

SEQ ID NO: 15 - protein, 350 aa, Paenibacillus polymyxa
MQALRWHGIKDLRLENIEQPAALPGKVKIKVEWCGICGSDLHEYVAGPIFIPENAQHPLTGEKSPIVMGHEFSGQFFDFGEGVTKIQVGDREVVEPVFACGECDACRQGKYNLCDKMGFLGLAGGGGGFSEYVAADEHMVHKIPESVSFEQGALVEPSAVALYAVRQIQLKVDDKAVVFGAGPIGLLVIEALNASGASEIYAEELSEERTAKAEDLGAIVLDPNTYDVVEELHKRTNGGVYVPYEVTEVPPVLTQAIESAKISGEIMIVIIFEKEALIKPNNIVMNERNLTGLICYDDVFPALISLMENGYFPADKLVIKRIKLVDVIEAAFESLLIEEYQVTILVSPHA

SEQ ID NO: 16 - DNA, 1053 bp, Paenibacillus polymyxa
  source 1..1053: mol_type = genomic DNA
atgcaggcgctgcgctggcacggcatcaaggacctgcggctggagaacatcgagcagcccgccgccctcccgggcaaggtgaagatcaaggtggaatggtgcggcatctgcggcagcgacctgcatgaatatgtcgccggcccgatcttcatccccgaaaacgcgcagcatccgctcacgggcgagaagtcgcccatcgtgatgggccatgagttctccggccagttcttcgacttcggcgaaggcgtgacgaaaatccaggtgggcgaccgcgaagtggtggagccggtcttcgcgtgtggcgaatgcgatgcgtgccggcagggcaaatataacctgtgcgataagatgggcttcctgggcctggccggcggcggcggcggcttctcggaatatgtcgccgcggatgagcatatggtgcacaaaatccccgagtccgtgtccttcgaacagggcgccctggtcgagccgtccgccgtcgccctctacgcggtccgccagatccagctgaaggtcgatgacaaggcggtggtcttcggcgccggccccatcggcctgctcgtcatcgaagcgctgaacgccagcggcgcgagcgaaatctatgcggaagagctcagcgaagagcgcaccgccaaagccgaagacctgggcgccatcgtgctcgaccccaacacgtacgatgtcgtcgaggaactccataagcgcacgaatggcggcgtctacgtcccctatgaggtcacggaagtcccgcccgtgctgacccaggccatcgagtccgccaagatctccggcgaaatcatgatcgtcatcatcttcgaaaaggaggccctcatcaagccgaacaacatcgtcatgaatgaacggaacctgacgggcctgatctgctacgacgatgtgttcccggccctgatctccctcatggagaatggctacttccccgccgacaagctggtcatcaaacggatcaagctggtggatgtcatcgaagcggccttcgagtcgctcctgatcgaggagtaccaggtgaccatcctcgtgtcgccgcacgcctga

SEQ ID NO: 17 - protein, 387 aa, Escherichia coli
MNLHEYQAKQLFARYGLPAPVGYACTTPREAEEAASKIGAGPWVVKCQVHAGGRGKAGGVKVVNSKEDIRAFAENWLGKRLVTYQTDANGQPVNQILVEAATDIAKELYLGAVVDRSSRRVVFMASTEGGVEIEKVAEETPHLIHKVALDPLTGPMPYQGRELAFKLGLEGKLVQQFTKIFMGLATIFLERDLALIEINPLVITKQGDLICLDGKLGADGNALFRQPDLREMRDQSQEDPREAQAAQWELNYVALDGNIGCMVNGAGLAMGTMDIVKLHGGEPANFLDVGGGATKERVTEAFKIILSDDKVKAVLVNIFGGIVRCDLIADGIIGAVAEVGVNVPVVVRLEGNNAELGAKKLADSGLNIIAAKGLDAAQQVVAAVEGK

SEQ ID NO: 18 - DNA, 2036 bp, Escherichia coli
  source 1..2036: mol_type = genomic DNA
atgaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataatgtccattttaatcgataaaaacaccaaggttatctgccagggctttaccggtagccaggggactttccactcagaacaggccattgcatacggcactaaaatggttggcggcgtaaccccaggtaaaggcggcaccacccacctcggcctgccggtgttcaacaccgtgcgtgaagccgttgctgccactggcgctaccgcttctgttatctacgtaccagcaccgttctgcaaagactccattctggaagccatcgacgcaggcatcaaactgattatcaccatcactgaaggcatcccgacgctggatatgctgaccgtgaaagtgaagctggatgaagcaggcgttcgtatgatcggcccgaactgcccaggcgttatcactccgggtgaatgcaaaatcggtatccagcctggtcacattcacaaaccgggtaaagtgggtatcgtttcccgttccggtacactgacctatgaagcggttaaacagaccacggattacggtttcggtcagtcgacctgtgtcggtatcggcggtgacccgatcccgggctctaactttatcgacattctcgaaatgttcgaaaaagatccgcagaccgaagcgatcgtgatgatcggtgagatcggcggtagcgctgaagaagaagcagctgcgtacatcaaagagcacgttaccaagccagttgtgggttacatcgctggtgtgactgcgccgaaaggcaaacgtatgggccacgcgggtgccatcattgccggtgggaaagggactgcggatgagaaattcgctgctctggaagccgcaggcgtgaaaaccgttcgcagcctggcggatatcggtgaagcactgaaaactgttctgaaataa

SEQ ID NO: 19 - protein, 451 aa, Porphyromonas gingivalis
MELKEMVSLARKAQKEYQATHNQEAVDNICRAAAKVIYENAAILAREAVDETGMGVYEHKVAKNQGKSKGVWYNLHNKKSIGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAVSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 20 - DNA, 1356 bp, Porphyromonas gingivalis
  source 1..1356: mol_type = genomic DNA
atggaaatcaaagaaatggtgagccttgcacgcaaggctcagaaggagtatcaagctacccataaccaagaagcagttgacaacatttgccgagctgcagcaaaagttatttatgaaaatgcagctattctggctcgcgaagcagtagacgaaaccggcatgggcgtttacgaacacaaagtggccaagaatcaaggcaaatccaaaggtgtttggtacaacctccacaataaaaaatcgattggtatcctcaatatagacgagcgtaccggtatgatcgagattgcaaagcctatcggagttgtaggagccgtaacgccgacgaccaacccgatcgttactccgatgagcaatatcatctttgctcttaagacctgcaatgccatcattattgccccccaccccagatccaaaaaatgctctgcacacgcagttcgtctgatcaaagaagctatcgctccgttcaacgtaccggaaggtatggttcagatcatcgaagaacccagcatcgagaagacgcaggaactcatgggcgccgtagacgtagtagttgctacgggtggtatgggcatggtgaagtctgcatattcttcaggaaagccttctttcggtgttggagccggtaacgttcaggtgatcgtggatagcaacatcgatttcgaagctgctgcagaaaaaatcatcaccggtcgtgctttcgacaacggtatcatctgctcaggcgaacagagcatcatctacaacgaggctgacaaggaagcagttttcacagcattccgcaaccacggtgcatatttctgtgacgaagccgaaggagatcgggctcgtgcagctatcttcgaaaatggagccatcgcgaaagatgtagtaggtcagagcgttgccttcattgccaagaaagcaaacatcaatatccccgagggtacccgtattctcgttgttgaagctcgcggcgtaggagcagaagacgttatctgtaaggaaaagatgtgtcccgtaatgtgcgccctcagctacaagcacttcgaagaaggtgtagaaatcgcacgtacgaacctcgccaacgaaggtaacggccacacctgtgctatccactccaacaatcaggcacacatcatcctcgcaggatcagagctgacggtatctcgtatcgtagtgaatgctccgagtgccactacagcaggcggtcacatccaaaacggtcttgccgtaaccaatacgctcggatgcggatcatggggtaataactctatctccgagaacttcacttacaagcacctcctcaacatttcacgcatcgcaccgttgaattcaagcattcacatccccgatgacaaagaaatctgggaactctaa

SEQ ID NO: 21 - protein, 371 aa, Porphyromonas gingivalis
MQLFKLKSVTHHFDTFAEFAKEFCLGERDLVITNEFIYEPYMKACQLPCHFVMQEKYGQGEPSDEMMNNILADIRNIQFDRVIGIGGGTVIDISKLFVLKGLNDVLDAFDRKIPLIKEKELIIVPTTCGTGSEVTNISIAEIKSRHTKMGLADDAIVADHAIIIPELLKSLPFHFYACSAIDALIHAIESYVSPKASPYSRLFSEAAWDIILEVFKKIAEHGPEYRFEKLGEMIMASNYAGIAFGNAGVGAVHALSYPLGGNYHVPHGEANYQFFTEVFKVYQKKNPFGYIVELNWKLSKILNCQPEYVYPKLDELLGCLLTKKPLHEYGMKDEEVRGFAESVLKTQQRLLANNYVELTVDEIEGIYRRLY

SEQ ID NO: 22 - DNA, 1114 bp, Porphyromonas gingivalis
  source 1..1114: mol_type = genomic DNA
atgcaacttttcaaactcaagagtgtaacacatcactttgacacttttgcagaatttgccaaggaattctgtcttggagaacgcgacttggtaattaccaacgagttcatctatgaaccgtatatgaaggcatgccagctcccctgccattttgttatgcaggagamtatgggcaaggcgagccttctgacgaaatgatgaataacatcttggcagacatccgtaatatccagttcgaccgcgtaatcggtatcggaggaggtacggttattgacatctctaaacttttcgttctgaaaggattaaatgatgtactcgatgcattcgaccgcaaaatacctcttatcaaagagaaagaactgatcattgtgcccacaacatgcggaacgggtagcgaggtgacgaacatttctatcgcagaaatcaaaagccgtcacaccaaaatgggattggctgacgatgccattgttgcagaccatgccatcatcatacctgaacttctgaagagcttgcctttccacttctacgcatgcagtgcaatcgatgctcttatccatgccatcgagtcatacgtatctcctaaagccagtccatattctcgtctgttcagtgaggcggcttgggacattatcctggaagtattcaagaaaatcgccgaacacggccctgaataccgcttcgaaaagctgggagaaatgatcatggccagcmctatgccggtatagccttcggaaatgcaggagtaggagccgtccacgcactatcctacccgttgggaggcaactatcacgtgccgcatggagaagcaaactatcagttcttcacagaggtattcaaagtataccaaaagaagaatcctttcggctatatagtcgaactcaactggaagctctccaagatactgaactgccagcccgaatacgtatatccgaagctggatgaacttctcggatgccttcttaccaagaaacctttgcacgaatacggcatgaaggacgaagaggtaagaggctttgcggaatcagtgcttaagacacagcaaagattgctcgccaacaactacgtagagcttactgtagatgagatcgaaggtatctacagaagactctactaa

SEQ ID NO: 23 - protein, 431 aa, Porphyromonas gingivalis
MKDVLAEYASRIVSAEEAVKHIKNGERVALSHAAGVPQSCVDALVQQADLFQNVEIYHMLCLGEGKYMAPEMAPHFRHITNFVGGNSRKAVEENRADFIPVFFYEVPSMIRKDILHIDVAIVQLSMPDENGYCSFGVSCDYSKPAAESAHLVIGEINRQMPYVHGDNLIHISKLDYIVMADYPIYSLAKPKIGEVEEAIGRNCAELIEDGATLQLGIGAIPDAALLFLKDKKDLGIHTEMFSDGVVELVRSGVITGKKKTLHPGKMVATFLMGSEDVYHFIDKNPDVELYPVDYVNDPRVIAQNDNMVSINSCIEIDLMGQVVSECIGSKQFSGTGGQVDYVRGAAWSKNGKSIMAIPSTAKNGTASRIVPIIAEGAAVTTLRNEVDYVVTEYGIAQLKGKSLRQRAEALIAIAHPDFREELTKHLRKRFG

SEQ ID NO: 24 - DNA, 1296 bp, Porphyromonas gingivalis
  source 1..1296: mol_type = genomic DNA
atgaaagacgtattagcggaatatgcctcccgaattgtttcggccgaagaagccgtaaaacatatcaaaaatggagaacgggtagctttgtcacatgctgccggagttcctcagagttgtgttgatgcactggtacaacaggccgaccttttccagaatgtcgaaatttatcacatgctttgtctcggcgaaggaaaatatatggcacctgaaatggcccctcacttccgacacataaccaattttgtaggtggtaattctcgtaaagcagttgaggaaaatagagccgacttcattccggtattcttttatgaagtgccatcaatgattcgcaaagacatccttcacatagatgtcgccatcgttcagctttcaatgcctgatgagaatggttactgtagttttggagtatcttgcgattatagcaaaccggcagcagaaagcgctcatttagttataggggaaatcaaccgtcaaatgccatatgtacatggcgacaacttgattcacatatcgaagttggattacatcgtgatggcagactaccctatctattctcttgcaaagcccaaaatcggagaagtagaagaagctatcgggcgtaattgtgccgagcttattgaagatggtgccacactccaactcggtatcggcgcgattcctgatgcagccctgttattcctcaaggacaaaaaagatctggggatccataccgagatgttctccgatggtgttgtcgaattagttcgcagtggagtaattacaggaaagaaaaagacacttcaccccggaaagatggtcgcaaccttcttaatgggaagcgaagacgtatatcatttcatcgacaaaaatcccgatgtagaactttatccggtagattacgtcaatgatccgcgagtaatcgctcaaaatgataatatggtcagcatcaatagctgtatcgaaatcgatcttatgggacaagtcgtgtccgaatgtataggaagcaagcaattcagcggaaccggcggtcaagtagattatgttcgtggagcagcatggtctaaaaacggcaaaagcatcatggcaattccctcaacagccaaaaacggtactgcatctcgaattgtacctataattgcagagggagctgctgtaacaaccctccgcaacgaagtcgattacgttgtaaccgaatacggtatagcacaactcaaaggaaagagtttgcgccagcgagcagaagctcttattgccatagcccacccggatttcagagaggaactaacgaaacatctccgcaaacgtttcggataa

SEQ ID NO: 25 - protein, 468 aa, Clostridium beijerinckii
MNKDTLIPTTKDLKLKTNVENINLKNYKDNSSCFGVFENVENAINSAVHAQKILSLHYTKEQREKIITEIRKAALENKEVLATMILEETHMGRYEDKILKHELVAKYTPGTEDLTTTAWSGDNGLTVVEMSPYGVIGAITPSTNPTETVICNSIGMIAAGNAVVFNGHPGAKKCVAFAIEMINKAIISCGGPENLVTTIKNPTMESLDAIIKHPLIKLLCGTGGPGMVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYFINKKWVGKDAKLFSDEIDVESPSNIKCIVCEVNANHPFVMTELMMPILPIVRVKDIDEAVKYTKIAEQNRKHSAYIYSKNIDNLNRFEREIDTTIFVKNAKSFAGVGYEAEGFTTFTIAGSTGEGITSARNFTRQRRCVLAG

SEQ ID NO: 26 - DNA, 2696 bp, Clostridium beijerinckii
  source 1..2696: mol_type = genomic DNA
tctagaggttcatcatcatccataggaaataatacacgtatttcataatcatcgctattaatatattcaattaatttttcaggagtctttactctctcccaaagaattaactcagttgatctggattcaccaatttcaataatctagcggtatttgaaggcctactaaattctctttctgttgataatatcaatatctttgcttttgtctctattttaggcacaatatcgcagatacaatttattattggcaacccacatttattgcagctctcatataacttagtaatttgcttaactttaaattcagactccattttacctccattattagttggttagtgtgtcatatcttcttgctattactaactgattataacatatgtattcaatatatcactcctagttttcaaagcactggcaatacgaattacaaattaatttctggatttatgtcagtatttcattaataaaaggtcggacttttaagatacttgttttagctattgatcatatttattaaagactatgcatttaatgtataattataatgaatattatcaataatatttattttatattacaatcttacagtctttattctaaatttcactcaaataccaaacgagctttattcataaacaatatataacaataattccaaaataatacgatattttatctgtaacagccatataaaaaaaatatcatatagtcttgtcatttgataacgttttgtcttccttatatttactttttcggtttaataggttgattctgtaaattttagtgataacatatatttgatgacattaaaaatttaatatttcatataaatttttaatgtctattaatttttaaatcacaaggaggaatagttcatgaataaagacacactaatacctacaactaaagatttaaaattaaaaacaaatgttgaaaacattaatttaaagaactacaaggataattcttcatgtttcggagtattcgaaaatgttgaaaatgctataaacagcgctgtacacgcgcaaaagatattatcccttcattatacaaaagaacaaagagaaaaaatcataactgagataagaaaggccgcattagaaaataaagaggttttagctaccatgattctggaagaaacacatatgggaaggtatgaagataaaatattaaagcatgaattagtagctaaatatactcctggtacagaagatttaactactactgcttggtcaggtgataatggtcttacagttgtagaaatgtctccatatggcgttataggtgcaataactccttctacgaatccaactgaaactgtaatatgtaatagcatcggcatgatagctgctggaaatgctgtagtatttaacggacacccaggcgctaaaaaatgtgttgcttttgctattgaaatgataaataaagcaattatttcatgtggcggtcctgagaatttagtaacaactataaaaaatccaactatggaatccctagatgcaattattaagcatcctttaataaaacttctttgcggaactggaggtccaggaatggtaaaaaccctcttaaattctggcaagaaagctataggtgctggtgctggaaatccaccagttattgtagatgataccgctgatatagaaaaggctggtaagagtatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatagatttagtattacaaaaaaataatgaaactcaagaatactttataaacaaaaaatgggtaggaaaagatgcaaaattattctcagatgaaatagatgttgagtctccttcaaatattaaatgcatagtctgcgaagtaaatgcaaatcatccatttgtcatgacagaactcatgatgccaatattaccaattgtaagagttaaagatatagatgaagctgttaaatatacaaagatagcagaacaaaatagaaaacatagtgcctatatttattctaaaaatatagacaacctaaatagatttgaaagagaaattgatactactatttttgtaaagaatgctaaatcttttgctggtgttggttatgaagctgaaggatttacaactttcactattgctggatctactggtgaaggcataacctctgcaagaaattttacaagacaaagaagatgtgtacttgccggctaacttcttgctaaatttatacatttattcacataactttaatatgcaatgttcccacaaaatattaaaaactatttagaagggagatattaaatgaataaattagtaaaattaacagatttaaagcgcattttcaaagatggtatgacaattatggttgggggttttttagattgtggaactcctgaaaatattatagatatgctagttgatttaaatataaaaaatctgactattataagcaatgatacagcttttcctaataaaggaataggaaaacttattgtaaatggtcaagtttctaaagtaattgcttcacatattggaactaatcctgaaactgggaaaaaaatgagctctggtgaacttaaagttgagctttctccacaaggaacactgatcgaaagaattc

SEQ ID NO: 27 - protein, 348 aa, Saccharomyces cerevisiae
MSIPETQKAIIFYESNGKLEHKDIPVPKPKPNELLINVKYSGVCHTDLHAWHGDWPLPTKLPLVGGHEGAGVVVGMGENVKGWKIGDYAGIKWLNGSCMACEYCELGNESNCPHADLSGYTHDGSFQEYATADAVQAAHIPQGTDLAEVAPILCAGITVYKALKSANLRAGHWAAISGAAGGLGSLAVQYAKAMGYRVLGIDGGPGKEELFTSLGGEVFIDFTKEKDIVSAVVKATNGGAHGIINVSVSEAAIEASTRYCRANGTVVLVGLPAGAKCSSDVFNHVVKSISIVGSYVGNRADTREALDFFARGLVKSPIKVVGLSSLPEIYEKMEKGQIAGRYVVDTSK

SEQ ID NO: 28 - DNA, 3627 bp, Saccharomyces cerevisiae
  source 1..3627: mol_type = genomic DNA
gatccttcaatatgcgcacatacgctgttatgttcaaggtcccttcgtttaagaacgaaagcggtcttccttttgagggatgtttcaagttgttcaaatctatcaaatttgcaaatccccagtctgtatctagagcgttgaatcggtgatgcgatttgttaattaaattgatggtgtcaccattaccaggtctagatataccaatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacacaatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtcaggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataagcggatctcctatgccttcacgatttatagtttccattatcaagtatgcctatattagtatatagcatctttagatgacagtgttcgaagtttcacgaataaaagataatattctactttttgctcccagcgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgtctccctcgatatttctcattttccttcgcatgccagcattgaaatgatcgaagttcaatgatgaaacggtaattcttctgtcatttactcatctcatctcatcaagttatataattctatacggatgtaatttttcacttttcgtcttgacgtccaccctataatttcaattattgaaccctcacaaatgatgcactgcaatgtacacaccctcatatagtttctcagggcttgatcagggttccgtagatgggaatttgagaagtataagggagataacggtaatgttatattgagaaaatgaaagatgaactcagtattgacaggatctaaaaccaaaatatagcgggcctatacaggaagtagtatttgtaaaagtaaaccatgttgctagtacgaacgacttccctgaatgtgtcaaggatgccagtgccatgcctcgccagaggaataggcatcctcaagggcaaatatagactagcgaacctgatgaatgcccaacctcagtgagacatgtgtcgagcgagatccagcaaaaggatcagcaggcaggagagtcaaacaccgccaccgatactggtgttattcacaaatcagatgaagaaactctgatatatttcgataatgtttacgctagaaccacctcggtttggaatccaacactgtggtacaatctcctgctaagaaaccagtcacgggatgcagtgagggagaaaataaggaatttagccagtccacccaataaccccatttatggactggagttgaagtctaccattccagtgaaaagggatggtggtgtatttgccacatttgtcgttccacccaagtatacaaaagctcaggtaaattctctaatccaacagaatacagccagagaatcctctaaaaacttactctcgtacttcactcgagcttctgccttcccagtaaagggttcgccttggattgaggatttaagaagactaccaagtaccactatagtcatcaagttccaagggcccgccttaacagaagaagaaatatactctttattcagaagatatgggacaattattgatattttccctcccactgctgctaacaacaatgtggcaa

SEQ ID NO: 29 - protein, 553 aa, Methylococcus capsulatus
MRETIPPRTGADLLVDSLQALGVEYVFGVPGGAILPILNVLADRGPRFIVCRDETGAAFMAQSWGRITGRPGVVLTTSGPGLINAVCGVATATEDRDPLVVITGQVPRAVQFKQSHMNLDSVGLFAPITKWSVEVEEPNTVSEILVNAFRTAQTPCAGAVHVSVPNDMLTAPVTAQALAPAEPAVWGTAPAAVVERAASLLNDAKAPAILLGLRASTPGAAAAVRRFLERHPLPVAMTFEAAGTLSRDLVDQFVGRVGYVLNQPGDEVLRQADLVLTIGYDPIEYEPSAWISPQSQAIHLDALPAAVDRAYHPAAELVGDIAANLAALGSLLRIEDRAGRPAVAAARRRLLEEQARGAALTGMPIHPLRFIHDLRATLDDEATVTCDVGAHEIWMARYFFCYAPRHLLFSMGHQTMGVALPWAIGAALARPGKKVVSVSGDGSFLMTCMELETAVRLKLPIVHIVWKDGGYNLIHSLQMRDYGRSFGAEFGPTDFVKLAEAFGAIGYRIESADGIVPVLNRALAADAPVLIEVPIDYSDNVHLVEAIDASAQH

SEQ ID NO: 30 - DNA, 1662 bp, Methylococcus capsulatus
  source 1..1662: mol_type = genomic DNA
atgcgtgaaacgatacctccccgcaccggcgccgacctgctggtcgactccctccaggcgctgggcgtcgaatacgtcttcggcgtgcccggcggcgcgatactcccgatcctgaacgtgctggccgaccgcggcccgcgcttcatcgtttgccgggacgaaaccggcgccgccttcatggcccagtcctggggccggatcaccggccggcccggcgtggtgctcaccacctccggccccggcctcatcaacgccgtctgtggcgtcgctaccgccacagaggaccgcgacccgctggtcgtcatcaccggccaggtgccgcgggccgtgcaattcaagcagagccacatgaacctggattcggtcggcctgttcgcgccgatcaccaaatggagcgtcgaggtcgaggaaccgaatactgtatcggaaatcctggtcaacgccttccgcaccgcgcagacgccgtgcgccggagccgtccacgtctcggtaccgaacgacatgctcaccgcgccggtcaccgcgcaggccctggcgccggccgaacccgccgtctggggaacggccccggccgccgtcgtcgaacgcgcggcgtccctgctgaacgatgccaaagccccggccatcctgctcggattgcgggccagcacacctggagcggcggcggcggtccggcgtttcctggagcggcatccgctgccggtggcgatgaccttcgaagccgccggcaccctgtcccgcgatctggtcgatcagttcgtcggccgggtcggctacgtgctcaaccagccgggcgacgaggtgctgcgccaagccgatctggtactcacgatcggctacgacccgatcgaatacgaaccttccgcctggatctcaccgcagtcgcaggcgatccacctggatgccctgcccgccgccgtcgaccgggcctaccaccctgccgccgaactggtcggcgacatcgccgccaacctggccgcgctcggcagcctgctccgaatcgaggatcgagccggacgccccgccgtcgccgcggcgcggcggcgtctgctggaggagcaagcccgcggcgcagcactgaccggtatgccgatccaccccttgcgcttcattcacgaccttcgggccacgctggacgacgaggcgacggtgacctgcgacgtcggcgcccacgagatctggatggcccgctacttcttctgctacgccccgcgtcacctgctgttcagcatgggccaccagaccatgggcgtcgccctgccctgggccatcggcgcggccctggcccggcccggcaagaaagtggtttcggtatccggcgacggctccttcctcatgacctgcatggaactggaaaccgcggtgcgcctcaaactgccgatcgtgcacatcgtctggaaagacggcggctacaacctgatccacagcctgcagatgcgcgactatgggcgcagcttcggcgccgagttcggccccaccgacttcgtcaaactggcggaggccttcggcgcgatcgggtaccggatcgagtccgcggacgggatcgtccctgtgctgaaccgggcgctcgcggccgacgcgccggtgctgatcgaagtgcccatcgactacagcgacaacgtccacctggtcgaggcgatcgacgcctcggcgcagcactga

SEQ ID NO: 31 - protein, 338 aa, Methylococcus capsulatus
MQIYYDKDADLSIIQGKKVAIIGYGSQGHAHANNLKDSGVQVVVGLRPGSASAKKAENAGLAVASVEDAVKQADVIMILAPDEHQARLYNEQIAPNIKQGAALAFAHGFNIHFEQITPRADLDVIMIAPKGPGHLVRSTYTQGGGVPSLIAVYQNASGRAKELALSYASANGGGRAGIIETTFREETETDLFGEQAVLCGGATALVQAGFETLVEAGYAPEMAYFECLHELKLIVDLMYEGGIANMRYSISNTAEYGDLTRGPRIVTEQTKQEMKKILREIQTGEFAREFILENQAGAATLKAKRRLGREHLIESVGARLRDMMPWIKANRIVDTSKN

SEQ ID NO: 32 - DNA, 1017 bp, Methylococcus capsulatus
  source 1..1017: mol_type = genomic DNA
atgcagatttactacgacaaagacgccgacctttccatcatccagggaaagaaggttgccatcatcggctacggctcgcagggccacgcccacgccaacaacctcaaggattccggagtgcaggtcgtggtggggctgcgtccgggttcggcttccgccaagaaggccgagaacgccggcctcgcggtcgcctcggtcgaggatgcggtcaaacaggcggacgtcatcatgatcctggcgccggacgagcatcaggcccgcctctacaatgaacagatcgcgccgaacatcaagcagggcgccgccctcgccttcgcccacggcttcaacatccacttcgagcagatcaccccgcgcgccgacctcgacgtgatcatgatcgcgcccaagggtcccggccatctggtacgttccacctacacccagggcggcggcgtgccctcgctgatcgccgtgtaccagaatgccagcgggcgcgccaaggaactcgcgctgtcctatgcttcggccaatggcggcggtcgggctggtatcatcgagaccaccttccgcgaagagaccgaaaccgatctgttcggcgaacaggccgtcctgtgtggcggcgccaccgcactggtgcaggcgggtttcgagacgctggtcgaagccggttatgcgcccgagatggcctatttcgagtgtctgcacgaactcaagctgatcgtcgacctgatgtacgaaggcggcatcgccaacatgcgttattcgatctccaatacggcagagtacggcgacctgacccgtggtccgcgcatcgtcaccgagcagaccaagcaggaaatgaagaaaatcctgcgcgagatccagaccggcgaattcgcccgtgagttcattttggaaaaccaggccggagccgccaccctgaaagcgaaacgccgtctcggccgagagcatctcatcgagagcgtgggcgccaggctgcgcgacatgatgccgtggatcaaggccaaccgcattgtggacacgagcaagaactga

SEQ ID NO: 33 - protein, 562 aa, Methylococcus capsulatus
MTDKHPRPHSSQVVDGMERAPSRAMLHAVGFADADFAKPQIGIASTWAMVTPCNMHINKLAEDAARGVDGGGGKAVIFNTITISDGISMGTEGMKYSLVSREVIADSIETVVACQGYDGVVAIGGCDKNMPGCLIALARLNRPAVFVYGGTILPGCHDGKKLDVVSVFEAVGARANHRIDDAELHAIESNAIPGPGSCGGMYTANTMASAIEALGMSLPGSSAQVAISRAKELDCERAGAQVLKLLDLGLKPRDIMTKKAFENAITVVIALGGSTNAVLHLLAMANACGVDLKLDDFTRIGRKVPMLADLKPSGRYSMAELVEIGGIQPLMKTLLDAGLLHGDCMTVTGKTLEENLADAPDYPAGQDMIRSLDNPIKKDSHLVILKGNLAPEGAVAKITGKEGLSFTGTARVFDCEEAALTAILDGTIVKGDVIVIRYEGPKGGPGMREMLSPTSAVMGKGLGKEVALITDGRFSGGTHGFVVGHITPEAYTGGPLAIVRDGDTITIDAETRELSLHVTDDEIGRRLAQWTQPAPRYTKGVLAKYARLVSPASEGAVTDDGL

SEQ ID NO: 34 - DNA, 1689 bp, Methylococcus capsulatus
  source 1..1689: mol_type = genomic DNA
atgaccgacaagcacccccgtccccattcgtcccaggtcgtcgacggcatggagcgcgccccgagccgcgcgatgctgcacgccgtcggcttcgccgatgccgacttcgccaaaccgcagatcggcatcgcttccacctgggcgatggtgacgccgtgcaacatgcacatcaacaagctcgccgaggacgcagcacgcggcgtcgacggcggcggcggcaaggcagtgatcttcaacaccatcaccatttccgacggcatctcgatgggcaccgaaggaatgaaatactccctcgtgtcgcgggaagtcatcgccgactcgatcgaaaccgtggtggcctgtcagggttatgacggcgtggtcgccatcggcggctgcgacaagaacatgcccggctgcctgatcgccctcgcccgcctcaaccgtccggcggtgttcgtctatggcggcaccatcctgccgggctgccacgacggcaagaagctggacgtggtgtcggtgttcgaagcggtcggcgcccgcgccaaccaccgcatcgacgatgccgaactgcacgccatcgaatccaatgccatccccggtccgggctcctgcggtggcatgtataccgccaacaccatggcctccgccatcgaggcattagggatgagcctgccgggcagttcggcccaggtggccatttcccgcgccaaggaactggattgcgagcgggccggcgcgcaggtcctcaagctcctggacctggggctcaaaccccgcgacatcatgaccaagaaggcgttcgagaacgccatcacggtggtgatcgccctgggcggctccaccaacgccgtgctgcacctcctggccatggccaacgcctgcggcgtcgacctgaagctcgacgatttcacccgcatcgggcgcaaagtgccgatgctggcggatctgaaacccagcggcagatactccatggccgaactggtggaaatcggcggcatccagccgctgatgaagaccttgctggacgcgggactcctgcacggcgactgcatgaccgtaaccggcaagaccctggaagaaaacctggccgacgcgcccgactacccggccggacaagacatgatccggtcgctggacaaccccatcaaaaaggacagccatctggtgatcctcaagggcaacctggcgccggaaggcgcggtcgccaagatcaccggcaaggaaggactgagcttcaccggcaccgcccgcgtattcgactgcgaggaagcggcgctcacggccatcctcgacggcacgatcgtgaaaggcgacgtcatcgtcatccgctatgaaggccccaagggcggccccggcatgcgcgagatgctctcgccgacctcggcggtcatgggcaagggattgggcaaggaggtcgccctcatcaccgacggccgcttttccggcggcacccacggcttcgtggtcggccacatcacgccggaagcctacaccggcggccccctggcgatcgtccgggacggcgataccatcaccatcgacgccgagacccgcgaattgagcctgcacgtcaccgacgatgaaatcggccggcgcctggcgcagtggactcaaccggcgccgcgctacaccaagggcgtgctggccaaatacgccaggttggtgagcccggcctcggaaggcgccgtcaccgacgacggcctctga

SEQ ID NO: 35 - protein, 549 aa, Methylococcus capsulatus
MGTVEPGAIGQHLLACLYQAGVGHIFGVPGDYVLGFYDLMAKGPVRHIGTTREDTAAFAADGYARCRGMGALAVTYGVGALNTVNAVAGAYAESSPVVVISGAPGVREQREDPLIHHRFGPFRFQREIFERITCAAVVLDDPVIAFRQVERALAAARQHCKPVYIEIPADRVMAPGYPIPQETPETPSSDDSALAEAVAEAAELLGRAVSPVILAGVELHRRGLQDALVGLVEQARLPVAATLTGKSVFAERHPAYLGVYEGAMSTENARYMVEQSDLLLMLGVTLNDVDTGIYTARLDPQRIVRAAQNEVVIRHHRYPRVLLADFVTALARSVKARGEAFPMPAGPEPWDFPAPDRPMTIARLVERLDRALTSDMIVVCDVGDCLFAATDLRVHERSEFLASAFYTSMGFAVPAALGAQIARPDHRALILVGDGAFQMTGTELSTHARLGLAPIVVVLDNRGYSTERFILDGAFNDIADWRFHRLGEVFGPLQGYDAPDEAAFENALSEALVNRNMPSLINVRLSPGDASIAMKRLAGHLQCRVKGEG

SEQ ID NO: 36 - DNA, 1650 bp, Methylococcus capsulatus
  source 1..1650: mol_type = genomic DNA
atgggcacggttgagcctggcgctatcggacaacatctgctcgcctgcctttaccaggcgggcgtcgggcacatcttcggcgttcccggcgattacgtgctgggcttctatgatctgatggccaaaggtcccgtccggcatatcgggaccacgcgggaggacaccgccgccttcgccgccgacggctatgcccgctgccggggcatgggcgcgctggcggtgacttacggggtcggtgcgctcaacaccgtcaacgccgtcgccggcgcctatgcggaatcctcgccggtggtggtcatcagcggtgcgccgggggtgcgcgagcaaagggaagacccgttgatccaccaccgcttcgggccgttccggttccagcgcgagatattcgaacggatcacctgcgccgccgtggtgctggacgatccggtgatcgccttccggcaggtggagcgtgcgctcgcggccgcccgtcagcactgcaagccggtgtacatcgagattcccgccgaccgggtgatggcgccgggatatccgattccacaggaaaccccggaaacgccttccagcgacgattcggccctggcggaggcggtcgccgaggccgcggagctcctgggccgtgcggtgtcgccggtgatccttgcaggcgtcgagttgcaccggcgagggctccaggacgccctcgtcggcctcgtcgagcaggcgcgcctgccggtggcggcgaccttgaccggcaagtcggtgttcgccgagcgccatcccgcctatctgggggtgtacgagggtgcgatgagcacggaaaacgcgcgctacatggtcgagcagtccgacctcctgctgatgctcggggtcacgctgaacgatgtcgacacgggcatctacacggcgcgtctcgatccgcagcgcatcgtccgcgcagcccagaacgaggtcgtgattcgccatcaccgctatccccgcgtcctgctcgcggacttcgtcacggccctggcgcggtccgtcaaggcccggggcgaggcgtttccgatgccggcggggccggaaccgtgggactttcccgcgccggaccggccgatgacgatcgcccggctggtggagcggctcgaccgcgcgctgacctccgacatgatcgtagtgtgcgacgtcggcgactgcctgttcgcagccaccgacctgcgcgtgcacgagcgcagcgaattcctggcgtccgccttctatacctcgatggggttcgcggtgcccgccgccctcggggcccagatcgcccgtccggaccaccgggcgctgatcctggtcggcgacggtgccttccagatgaccggaacggagctgtcgacccatgcccgtctcggcctggcgcccatcgtggtggtgctcgacaatcgcggttacagcaccgagcgcttcatcctcgacggagccttcaacgacatcgccgactggcgcttccaccggctgggcgaggtgttcggccccctacagggctacgacgcgcccgacgaagcggcgttcgaaaacgcgctcagcgaagcgctggtcaaccgaaacatgccgagcctcatcaacgtccgtctttcccccggcgatgcctcgatagccatgaagcgtctcgccgggcatctgcagtgccgggtcaagggcgagggctga

SEQ ID NO: 37 - protein, 590 aa, Methylosinus trichosporium
MARKMTGAEMVVEALKDQGVEIIFGYPGGAVLPIYDALFHQEKVQHILVRHEQGAAHAAEGYARSSGKVGVLLVTSGPGATNTITGLTDALMDSIPVVCITGQVPTHLIGSDAFQECDTVGITRHCTKHNYLVKSVDDLPRILHEAFYVASSGRPGPVVIDIPKDVQFASGTYTGPRNVHHKTYQPKLEGDTESIRRAVKMMAAAKRPIFYTGGGVINSGPAASTLLRELVSLTGFPITSTLMGLGAYPGSGPNWLGMLGMHGTFEANNAMHDCDLMIAVGARFDDRITGRLDAFSPGSKKIHIDIDRSSINKNVKIDLPIVGDCGHVLESLVRVWRSEAMHAEKQPLDGWWKTIDHWRERKSLAFRNSDKVIKPQYAVQRLYALTKDRDPYITTEVGQHQMWAAQHYHFDEPNRWMTSGGLGTMGYGLPAAIGAQLAHPKSLVVDIAGEASILMNIQEMSTAIQYRLPVKVFILNNEYMGMVRQWQELLHGGRYSHSYSEALPDFVKLAEAFGGKGIRCSDPAELDSAILEMIDYDGPVIFDCLVEKNENCFPMIPSGKAHNDMLLADLGDDAGVELGSIIDEKGKMLV

SEQ ID NO: 38 - DNA, 1773 bp, Methylosinus trichosporium
  source 1..1773: mol_type = genomic DNA
atggccagaaaaatgaccggagcggaaatggtcgtcgaagccctgaaggatcagggcgtcgagattatcttcggctatcccggcggcgccgtgcttccgatctatgacgcgctcttccaccaggagaaggtgcagcacattctcgtgcgccacgagcagggcgccgcccatgcggccgagggctatgcgcgctcctccggcaaggtcggcgtgctgctggtcacctccggacccggcgccaccaacaccatcaccggcctcaccgatgcgctgatggactccattcccgtggtctgcatcaccggccaggtgccgacgcatctcatcggctcggacgcctttcaagagtgcgatacggtcggcatcacccgtcactgcaccaagcataattatctggtgaagagcgtcgacgatctgccgcgcattctgcacgaggccttctatgtcgcctcgagcgggcggccgggccctgtggtcatcgacatccccaaggatgtgcaattcgccagcggaacctataccggcccgcgcaacgtccatcacaagacctatcagcccaagctcgagggcgacacggagtctatccgccgcgccgtgaagatgatggccgccgccaagcggccgatcttctacaccggcggcggcgtcatcaattccggtcccgcggcctcgacgctgctgcgcgagctggtgtcgctgaccggctttccgatcacctcgaccttgatgggcctcggcgcctatccgggctccggccccaattggctcggcatgctcggcatgcacggcaccttcgaggccaataatgcgatgcatgattgcgatctgatgatcgccgtcggcgcgcgtttcgacgatcgcatcaccggacggctcgacgccttctcgcccggctcgaagaagatccacatcgatatcgatcgctcctcgatcaataagaatgtgaagatcgatctgccgatcgtcggcgactgcggccatgtgctggagagtctggtgcgcgtctggcgctccgaggcgatgcacgccgagaagcagccgctcgacggctggtggaagacgatcgaccattggcgcgagcgcaagtcgctcgccttccgcaattcggacaaggtgatcaagccgcaatacgccgtgcagcggctctatgcgctcaccaaggatcgcgatccctacatcacgacggaagtcggccagcatcagatgtgggccgcgcagcattatcatttcgacgagcccaatcgctggatgacttccggcgggctcggcaccatgggctatggtctgccggcggcgatcggcgcgcagctcgcgcatccgaaatcgctggtcgtcgacatcgccggcgaggcctcgatcctgatgaacattcaggagatgtcgacggcgatccaatatcggctgccggtgaaggtgttcatcctcaacaatgaatatatgggcatggtgcgccagtggcaggagctgctgcacggcgggcgctactcgcactcctattcggaggcgctgcccgatttcgtgaagctcgccgaagccttcgggggcaagggcatccgctgctcggacccggcggagctcgatagcgcgattctcgagatgatcgactatgacgggccggtgatcttcgattgtctcgtcgagaaaaacgagaattgcttcccgatgatcccgtcgggcaaggcgcataacgacatgctgctcgccgatctcggcgacgacgccggcgtcgagctcggctcgatcatcgacgagaagggcaagatgctggtgtga

SEQ ID NO: 39 - protein, 548 aa, Lactococcus lactis
MYTVGDYLLDRLHELGIEEIFGVPGDYNLQFLDQIISRKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENPTSNTSDQEILNKIQESLKNAKKPIVITGHEIISFGLENTVTQFISKTKLPITTLNFGKSSVDETLPSFLGIYNGKLSEPNLKEFVESADFILMLGVKLTDSSTGAFTHHLNENKMISLNIDEGKIFNESIQNFDFESLISSLLDLSGIEYKGKYIDKKQEDFVPSNALLSQDRLWQAVENLTQSNETIVAEQGTSFFGASSIFLKPKSHFIGQPLWGSIGYTFPAALGSQIADKESRHLLFIGDGSLQLTVQELGLAIREKINPICFIINNDGYTVEREIHGPNQSYNDIPMWNYSKLPESFGATEERVVSKIVRTENEFVSVMKEAQADPNRMYWIELVLAKEDAPKVLKKMGKLFAEQNKS

SEQ ID NO: 40 - DNA, 1647 bp, Lactococcus lactis
  source 1..1647: mol_type = genomic DNA
atgtacaccgtgggcgactatctgctggaccggctgcatgaactgggcatcgaggaaatcttcggcgtccccggcgactataacctgcagttcctggaccagatcatcagccgcaaggacatgaagtgggtgggcaacgccaacgagctgaacgcctcgtacatggcggacggctatgcccggaccaagaaggccgcggccttcctgaccaccttcggcgtcggcgaactgagcgccgtgaacggcctggcgggctcgtacgccgagaacctgccggtcgtggaaatcgtcggctcccccaccagcaaggtgcagaacgagggcaagttcgtccaccataccctggccgacggcgacttcaagcacttcatgaagatgcatgaaccggtgaccgcggcccgcaccctgctgaccgccgagaacgcgaccgtcgaaatcgaccgcgtgctgagcgcgctgctgaaggagcggaagccggtctatatcaacctgcccgtcgacgtggcggccgcgaaggccgagaagccgtccctgcccctgaagaaggaaaaccccacctcgaacacctccgaccaggagatcctgaacaagatccaggaaagcctgaagaacgccaagaagccgatcgtgatcaccggccacgagatcatctcgttcggcctggaaaacaccgtcacccagttcatctccaagaccaagctgccgatcaccaccctgaacttcggcaagagctcggtggacgagaccctgccctcgttcctgggcatctacaacggcaagctgtccgaaccgaacctgaaggagttcgtggaaagcgcggacttcatcctgatgctgggcgtcaagctgaccgactccagcaccggcgccttcacccaccatctgaacgagaacaagatgatctcgctgaacatcgacgagggcaagatcttcaacgaatccatccagaacttcgacttcgaaagcctgatctcgtccctgctggacctgtccggcatcgagtacaagggcaagtatatcgacaagaagcaggaagacttcgtcccgagcaacgcgctgctgtcgcaggaccgcctgtggcaggccgtggagaacctgacccagagcaacgagaccatcgtcgcggaacagggcacctcgttcttcggcgccagctcgatcttcctgaagccgaagtcgcacttcatcggccagcccctgtggggctccatcggctacaccttccccgccgcgctgggctcgcagatcgcggacaaggaatcccggcatctgctgttcatcggcgacggcagcctgcagctgaccgtgcaggagctgggcctggccatccgcgaaaagatcaacccgatctgcttcatcatcaacaacgacggctataccgtcgagcgggaaatccacggcccgaaccagtcgtacaacgacatccccatgtggaactattccaagctgccggagagcttcggcgccaccgaggaacgcgtcgtgtccaagatcgtccggaccgagaacgagttcgtcagcgtgatgaaggaagcccaggcggaccccaaccggatgtactggatcgagctggtgctggcgaaggaagacgccccgaaggtcctgaagaagatgggcaagctgttcgccgaacagaacaagagctga

SEQ ID NO: 41 - protein, 563 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVIRLIELKLPVFDAPESLIKQAQLTAATNAKQ

SEQ ID NO: 42 - DNA, 1692 bp, Saccharomyces cerevisiae
  source 1..1692: mol_type = genomic DNA
atgtcggaaatcaccctgggcaagtacctgttcgagcggctgaagcaggtcaacgtcaacaccatcttcggcctgcccggcgacttcaacctgagcctgctggacaagatctacgaggtcgacggcctgcgctgggccggcaacgcgaacgaactgaacgccgcgtacgccgcggacggctatgcccggatcaagggcctgtcggtcctggtgaccaccttcggcgtgggcgagctgtcggccctgaacggcatcgccggctcctacgcggaacacgtcggcgtgctgcatgtcgtgggcgtcccgagcatctcggcccaggcgaagcagctgctgctgcaccataccctgggcaacggcgacttcaccgtgttccaccgcatgtccgccaacatcagcgagaccacctcgatgatcaccgacatcgccaccgcgccgagcgaaatcgaccgcctgatccggaccaccttcatcacccagcggccgtcgtacctgggcctgcccgccaacctggtcgacctgaaggtgccgggcagcctgctggagaagcccatcgacctgtcgctgaagccgaacgaccccgaggccgaaaaggaagtcatcgacaccgtgctggaactgatccagaacagcaagaacccggtcatcctgtccgacgcctgcgcgagccgccacaacgtgaagaaggagacccagaagctgatcgacctgacccagttcccggccttcgtcacccccctgggcaagggctccatcgacgagcagcatccgcggtacggcggcgtctatgtgggcaccctgagcaagcaggacgtcaagcaggccgtggaaagcgcggacctgatcctgtcggtgggcgccctgctgtccgacttcaacaccggctccttcagctactcgtataagaccaagaacgtcgtggagttccattcggactacgtcaaggtgaagaacgcgaccttcctgggcgtccagatgaagttcgccctgcagaacctgctgaaggtgatcccggacgtcgtgaagggctataagtccgtcccggtgcccaccaagacccccgccaacaagggcgtcccggcgtcgacccccctgaagcaggaatggctgtggaacgagctgtccaagttcctgcaggaaggcgacgtgatcatctcggagaccggcacctccgcgttcggcatcaaccagaccatcttcccgaaggacgcctacggcatcagccaggtcctgtggggctcgatcggcttcaccaccggcgccaccctgggcgccgcgttcgccgcggaggaaatcgacccgaacaagcgcgtcatcctgttcatcggcgacggctccctgcagctgaccgtgcaggaaatcagcaccatgatccggtggggcctgaagccctacctgttcgtgctgaacaacgacggctataccatcgagaagctgatccacggcccgcatgcggaatacaacgagatccagacctgggaccacctggccctgctgcccgccttcggcgcgaagaagtatgaaaaccataagatcgccaccaccggcgagtgggacgcgctgaccaccgactccgagttccagaagaacagcgtcatccgcctgatcgagctgaagctgccggtgttcgacgcccccgaaagcctgatcaagcaggcgcagctgaccgccgcgaccaacgccaagcagtga

SEQ ID NO: 43 - protein, 635 aa, Saccharomyces cerevisiae
MAPVTIEKFVNQEERHLVSNRSATIPFGEYIFKRLLSIDTKSVFGVPGDFNLSLLEYLYSPSVESAGLRWVGTCNELNAAYAADGYSRYSNKIGCLITTYGVGELSALNGIAGSFAENVKVLHIVGVAKSIDSRSSNFSDRNLHHLVPQLHDSNFKGPNHKVYHDMVKDRVACSVAYLEDIETACDQVDNVIRDIYKYSKPGYIFVPADFADMSVTCDNLVNVPRISQQDCIVYPSENQLSDIINKITSWIYSSKTPAILGDVLTDRYGVSNFLNKLICKTGIWNFSTVMGKSVIDESNPTYMGQYNGKEGLKQVYEHFELCDLVLHFGVDINEINNGHYTFTYKPNAKIIQFHPNYIRLVDTRQGNEQMFKGINFAPILKELYKRIDVSKLSLQYDSNVTQYTNETMRLEDPTNGQSSIITQVHLQKTMPKFLNPGDVVVCETGSFQFSVRDFAFPSQLKYISQGFFLSIGMALPAALGVGIAMQDHSNAHINGGNVKEDYKPRLILFEGDGAAQMTIQELSTILKCNIPLEVIIWNNNGYTIERAIMGPTRSYNDVMSWKWTKLFEAFGDFDGKYTNSTLIQCPSKLALKLEELKNSNKRSGIELLEVKLGELDFPEQLKCMVEAAALKRNKK

SEQ ID NO: 44 - DNA, 1908 bp, Saccharomyces cerevisiae
  source 1..1908: mol_type = genomic DNA
atggcccccgtcaccatcgagaagttcgtcaaccaggaagagcggcatctggtgtccaaccggagcgcgaccatcccgttcggcgagtacatcttcaagcgcctgctgagcatcgacaccaagtcggtgttcggcgtgccgggcgacttcaacctgagcctgctggagtacctgtatagcccctcggtcgaatcggccggcctgcgctgggtgggcacctgcaacgaactgaacgccgcgtacgccgcggacggctactcccggtatagcaacaagatcggctgcctgatcaccacctatggcgtcggcgaactgtcggcgctgaacggcatcgcgggctccttcgccgagaacgtgaaggtcctgcacatcgtgggcgtcgccaagtcgatcgactcccgcagctcgaacttctcggaccggaacctgcaccatctggtcccgcagctgcatgactccaacttcaagggccccaaccacaaggtgtaccatgacatggtgaaggaccgcgtcgcgtgctccgtggcctatctggaggacatcgaaaccgcctgcgaccaggtggacaacgtcatccgggacatctacaagtatagcaagccgggttacatcttcgtccccgcggacttcgccgacatgtccgtgacctgcgacaacctggtgaacgtcccgcgcatcagccagcaggactgcatcgtgtacccctccgaaaaccagctgagcgacatcatcaacaagatcacctcgtggatctactccagcaagaccccggccatcctgggcgacgtcctgaccgaccggtatggcgtgagcaacttcctgaacaagctgatctgcaagaccggcatctggaacttctcgaccgtcatgggcaagtcggtgatcgacgaatccaacccgacctacatgggccagtataacggcaaggaaggcctgaagcaggtctacgagcacttcgaactgtgcgacctggtcctgcatttcggcgtggacatcaacgagatcaacaacggccactacaccttcacctataagccgaacgcgaagatcatccagttccatcccaactacatccgcctggtggacacccggcagggcaacgaacagatgttcaagggcatcaacttcgccccgatcctgaaggagctgtataagcgcatcgacgtcagcaagctgtcgctgcagtacgacagcaacgtgacccagtataccaacgagaccatgcggctggaagaccccaccaacggccagtcgtccatcatcacccaggtccacctgcagaagaccatgccgaagttcctgaaccccggcgacgtcgtggtctgcgagaccggctccttccagttcagcgtgcgcgacttcgcgttcccgagccagctgaagtacatctcgcagggcttcttcctgtccatcggcatggccctgcccgccgcgctgggcgtcggcatcgcgatgcaggaccactcgaacgcccatatcaacggcggcaacgtgaaggaagactacaagccgcggctgatcctgttcgaaggcgacggcgccgcgcagatgaccatccaggagctgtccaccatcctgaagtgcaacatcccgctggaagtcatcatctggaacaacaacggctacaccatcgagcgcgccatcatgggccccacccggagctataacgacgtgatgtcgtggaagtggaccaagctgttcgaagcgttcggcgacttcgacggcaagtacaccaactccaccctgatccagtgcccgagcaagctggccctgaagctggaggaactgaagaactcgaacaagcgctccggcatcgagctgctggaagtcaagctgggcgagctggacttccccgaacagctgaagtgcatggtggaggccgcggccctgaagcggaacaagaagtga

SEQ ID NO: 45 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 46 - DNA, 1692 bp, Saccharomyces cerevisiae
  source 1..1692: mol_type = genomic DNA
atgagcgagatcaccctgggcaagtacctgttcgagcggctgaagcaggtcaacgtcaacaccgtcttcggcctgcccggcgacttcaacctgagcctgctggacaagatctacgaggtcgaaggcatgcgctgggcgggcaacgccaacgagctgaacgccgcgtacgccgcggacggctatgcccggatcaagggcatgtcgtgcatcatcaccaccttcggcgtgggcgagctgtccgccctgaacggcatcgcgggcagctacgccgaacacgtcggcgtgctgcatgtcgtgggcgtcccgagcatctcggcccaggcgaagcagctgctgctgcaccataccctgggcaacggcgacttcaccgtgttccaccgcatgtccgcgaacatcagcgagaccaccgccatgatcaccgacatcgccaccgcgccggccgaaatcgaccgctgcatccggaccacctacgtcacccagcggcccgtgtatctgggcctgccggccaacctggtcgacctgaacgtgcccgcgaagctgctgcagaccccgatcgacatgtcgctgaagcccaacgacgccgagtccgaaaaggaagtcatcgacaccatcctggcgctggtcaaggacgccaagaacccggtgatcctggcggacgcctgctgctcccgccacgacgtcaaggccgagaccaagaagctgatcgacctgacccagttccccgccttcgtgaccccgatgggcaagggctccatcgacgaacagcatccgcggtacggcggcgtctatgtgggcaccctgagcaagcccgaagtcaaggaagccgtggaaagcgccgacctgatcctgtcggtcggcgccctgctgtccgacttcaacaccggctccttcagctactcgtataagaccaagaacatcgtggagttccacagcgaccacatgaagatccgcaacgccaccttccccggcgtccagatgaagttcgtgctgcagaagctgctgaccaccatcgccgacgccgcgaagggctacaagccggtcgcggtgcccgcccggaccccggcgaacgccgcggtccccgcctcgaccccgctgaagcaggaatggatgtggaaccagctgggcaacttcctgcaggaaggcgacgtcgtgatcgcggaaaccggcacctccgccttcggcatcaaccagaccaccttcccgaacaacacctacggcatcagccaggtgctgtggggctcgatcggcttcaccaccggcgccaccctgggcgccgcgttcgccgcggaggaaatcgacccgaagaagcgcgtcatcctgttcatcggcgacggcagcctgcagctgaccgtgcaggaaatctcgaccatgatccggtggggcctgaagccctacctgttcgtcctgaacaacgacggctataccatcgagaagctgatccacggcccgaaggcccagtacaacgaaatccagggctgggaccatctgtcgctgctgcccaccttcggcgccaaggactatgagacccatcgcgtggcgaccaccggcgaatgggacaagctgacccaggacaagtcgttcaacgacaactccaagatccggatgatcgagatcatgctgcccgtcttcgacgcgccgcagaacctggtggaacaggccaagctgaccgccgcgaccaacgcgaagcagtga

SEQ ID NO: 47 - protein, 1048 aa, Clostridium acetobutylicum
MKSEYTIGRYLLDRLSELGIRHIFGVPGDYNLSFLDYIMEYKGIDWVGNCNELNAGYAADGYARINGIGAILTTFGVGELSAINAIAGAYAEQVPVVKITGIPTAKVRDNGLYVHHTLGDGRFDHFFEMFREVTVAEALLSEENAAQEIDRVLISCWRQKRPVLINLPIDVYDKPINKPLKPLLDYTISSNKEAACEFVTEIVPIINRAKKPVILADYGVYRYQVQHVLKNLAEKTGFPVATLSMGKGVFNEAHPQFIGVYNGDVSSPYLRQRVDEADCIISVGVKLTDSTTGGFSHGFSKRNVIHIDPFSIKAKGKKYAPITMKDALTELTSKIEHRNFEDLDIKPYKSDNQKYFAKEKPITQKRFFERIAHFIKEKDVLLAEQGTCFFGASTIQLPKDATFIGQPLWGSIGYTLPALLGSQLADQKRRNILLIGDGAFQMTAQEISTMLRLQIKPIIFLINNDGYTIERAIHGREQVYNNIQMWRYHNVPKVLGPKECSLTFKVQSETELEKALLVADKDCEHLIFIEVVMDRYDKPEPLERLSKRFANQNNGYARINGIGAILTTFGVGELSAINAIAGAYAEQVPVVKITGIPTAKVRDNGLYVHHTLGDGRFDHFFEMFREVTVAEALLSEENAAQEIDRVLISCWRQKRPVLINLPIDVYDKPINKPLKPLLDYTISSNKEAACEFVTEIVPIINRAKKPVILADYGVYRYQVQHVLKNLAEKTGFPVATLSMGKGVFNEAHPQFIGVYNGDVSSPYLRQRVDEADCIISVGVKLTDSTTGGFSHGFSKRNVIHIDPFSIKAKGKKYAPITMKDALTELTSKIEHRNFEDLDIKPYKSDNQKYFAKEKPITQKRFFERIAHFIKEKDVLLAEQGTCFFGASTIQLPKDATFIGQPLWGSIGYTLPALLGSQLADQKRRNILLIGDGAFQMTAQEISTMLRLQIKPIIFLINNDGYTIERAIHGREQVYNNIQMWRYHNVPKVLGPKECSLTFKVQSETELEKALLVADKDCEHLIFIEVVMDRYDKPEPLERLSKRFANQNN

SEQ ID NO: 48 - DNA, 3147 bp, Clostridium acetobutylicum
  source 1..3147: mol_type = genomic DNA
atgaagtcggaatacaccatcggccgctatctgctggaccgcctgagcgagctgggcatccgccacatcttcggcgtccccggcgactacaacctgtcgttcctggactacatcatggagtataagggcatcgactgggtcggcaactgcaacgaactgaacgccggctacgccgcggacggctatgcccgcatcaacggcatcggcgcgatcctgaccaccttcggcgtcggcgagctgtccgccatcaacgccatcgcgggcgcctacgcggaacaggtgccggtcgtgaagatcaccggcatccccaccgccaaggtccgcgacaacggcctgtatgtgcaccataccctgggcgacggccgcttcgaccacttcttcgagatgttccgggaagtcaccgtggccgaggcgctgctgagcgaggaaaacgccgcgcaggaaatcgaccgcgtgctgatctcgtgctggcgccagaagcggccggtcctgatcaacctgcccatcgacgtgtacgacaagccgatcaacaagccgctgaagcccctgctggactataccatcagctcgaacaaggaagccgcgtgcgagttcgtcaccgagatcgtgccgatcatcaaccgcgccaagaagcccgtcatcctggcggactacggcgtgtaccggtatcaggtccagcacgtgctgaagaacctggcggagaagaccggcttcccggtcgccaccctgtcgatgggcaagggcgtgttcaacgaagcccatccgcagttcatcggcgtctacaacggcgacgtgtccagcccctatctgcgccagcgggtcgacgaggccgactgcatcatctcggtcggcgtgaagctgaccgactccaccaccggcggcttctcccacggcttcagcaagcgcaacgtgatccatatcgacccgttctccatcaaggccaagggcaagaagtacgcgcccatcaccatgaaggacgccctgaccgaactgacctcgaagatcgagcaccggaacttcgaagacctggacatcaagccgtacaagtccgacaaccagaagtatttcgcgaaggagaagcccatcacccagaagcgcttcttcgaacggatcgcccatttcatcaaggagaaggacgtcctgctggcggaacagggcacctgcttcttcggcgccagcaccatccagctgccgaaggacgcgaccttcatcggccagcccctgtggggctccatcggctacaccctgccggccctgctgggcagccagctggcggaccagaagcgtcgcaacatcctgctgatcggcgacggcgccttccagatgaccgcgcaggagatctcgaccatgctgcgcctgcagatcaagccgatcatcttcctgatcaacaacgacggctacaccatcgagcgcgccatccacggccgggaacaggtgtacaacaacatccagatgtggcggtatcataacgtcccgaaggtgctgggccccaaggaatgcagcctgaccttcaaggtccagtcggagaccgaactggagaaggccctgctggtcgccgacaaggactgcgagcacctgatcttcatcgaagtcgtgatggaccgctacgacaagccggagcccctggaacgcctgtccaagcggttcgccaaccagaacaacggctatgcgcggatcaacggcatcggcgccattttaaccaccttcggcgtgggcgagctgagcgcgatcaacgcgatcgccggcgcctacgcggagcaggtgccggtggtcaaaattaccggcatccccaccgcgaaggtgcgggacaacggcctgtacgtccatcacaccctgggcgacggccggttcgaccatttcttcgaaatgttccgggaggtgaccgtcgccgaggcgctgctgtcggaagagaacgcggcccaggagatcgaccgcgtcctgatcagctgctggcggcagaagcgccccgtgctgatcaacctgccgatcgacgtctatgacaagcccatcaacaagcccctgaagccgctgctggactacaccatctcgtccaacaaggaagccgcctgcgagttcgtcaccgaaatcgtccccatcatcaaccgcgcgaagaagccggtgatcctggccgactatggcgtctatcggtatcaggtgcagcatgtcctgaagaacctggccgaaaagaccggcttccccgtggccaccctgagcatgggcaagggcgtcttcaacgaggcgcacccccagttcatcggcgtgtataacggcgacgtgagctcgccgtacctgcggcagcgcgtggacgaagccgactgcatcatcagcgtcggcgtcaagctgaccgactcgaccaccggcggcttctcgcacggcttctcgaagcggaacgtcatccacatcgacccgttctcgatcaaggcgaagggcaagaagtatgccccgatcaccatgaaggacgcgctgaccgaactgaccagcaagatcgaacatcgcaacttcgaggacctggacatcaagccctacaagtcggacaaccagaagtacttcgccaaggaaaagccgattactcagaagcgcttcttcgagcgcatcgcgcacttcatcaaggaaaaggacgtcctgctggccgagcaaggcacctgcttcttcggtgcgtcgaccatccagctgcccaaggacgccaccttcatcggccagccgctgtggggctcgatcggctataccctgcccgcgctgctgggctcccagctggccgatcaaaaacgtcgcaatattttactgatcggcgacggcgcgttccagatgaccgcccaggagatcagcaccatgctgcggctgcagatcaagcccattatcttcctgattaacaacgacggctataccatcgaacgggcgatccacggccgcgagcaggtctataataatattcaaatgtggcggtatcataatgtgcccaaggtcctgggcccgaaggaatgctcgctgaccttcaaggtgcagagcgaaaccgagctggaaaaggccctgctggtcgccgataaggactgcgaacatctgatcttcatcgaggtggtcatggaccggtatgacaagcccgaacccctggaacggctgagcaagcgcttcgcgaaccagaacaactga

SEQ ID NO: 49 - protein, 360 aa, Saccharomyces cerevisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 50 - DNA, 1083 bp, Saccharomyces cerevisiae
  source 1..1083: mol_type = genomic DNA
atgtcttatcctgagaaatttgaaggtatcgctattcaatcacacgaagattggaaaaacccaaagaagacaaagtatgacccaaaaccattttacgatcatgacattgacattaagatcgaagcatgtggtgtctgcggtagtgatattcattgtgcagctggtcattggggcaatatgaagatgccgctagtcgttggtcatgaaatcgttggtaaagttgtcaagctagggcccaagtcaaacagtgggttgaaagtcggtcaacgtgttggtgtaggtgctcaagtcttttcatgcttggaatgtgaccgttgtaagaatgataatgaaccatactgcaccaagtttgttaccacatacagtcagccttatgaagacggctatgtgtcgcagggtggctatgcaaactacgtcagagttcatgaacattttgtggtgcctatcccagagaatattccatcacatttggctgctccactattatgtggtggtttgactgtgtactctccattggttcgtaacggttgcggtccaggtaaaaaagttggtatagttggtcttggtggtatcggcagtatgggtacattgatttccaaagccatgggggcagagacgtatgttatttctcgttcttcgagaaaaagagaagatgcaatgaagatgggcgccgatcactacattgctacattagaagaaggtgattggggtgaaaagtactttgacaccttcgacctgattgtagtctgtgcttcctcccttaccgacattgacttcaacattatgccaaaggctatgaaggttggtggtagaattgtctcaatctctataccagaacaacacgaaatgttatcgctaaagccatatggcttaaaggctgtctccatttcttacagtgctttaggttccatcaaagaattgaaccaactcttgaaattagtctctgaaaaagatatcaaaatttgggtggaaacattacctgttggtgaagccggcgtccatgaagccttcgaaaggatggaaaagggtgacgttagatatagatttaccttagtcggctacgacaaagaattttcagactag

SEQ ID NO: 51 - protein, 351 aa, Methylosinus trichosporium
MSTKAYAVASAEALFGPLAIERRALGPEDVEIDILYCGVCHSDLHTARSEWPGTRYPCVPGHEIVGRVTAVGAKVTKFSVGDLAAVGCMVDSCRRCLSCDDGLEQYCEHGFTATYNGPIYGSGENTFGGYSEKIVVDAHFVLAIHHSETQLAGVAPLLCAGITTWSPLKHWGVGPGKSVGIVGIGGLGHMGVKLAHALGAHVVAFTTSPSKRDAALALGADEVVVSTDPAAMAARAGSLDFILDTVAVAHDLDAYVNLLKRDGALVLVGVPATPHPSPSAGGLIFKRRQVAGSLIGGVKETQEMLDFCAERGIVADIETIAMQQIETAYARMLKNDVKYRFVIDMATLKAA

SEQ ID NO: 52 - DNA, 1056 bp, Methylosinus trichosporium
  source 1..1056: mol_type = genomic DNA
atgtccaccaaagcctatgccgttgcgtccgccgaggcgctcttcggcccgctcgcgatcgagcgccgcgcgctcgggcccgaggatgtagagatcgacatcctctattgcggcgtctgccattccgatctgcacacggcgcgcagtgaatggccgggcacgcgctacccatgcgtcccgggccacgagattgtcggccgcgtcaccgctgtcggcgcgaaggtgacgaaattttcggtcggcgatctcgccgccgtcggctgcatggtcgacagctgccggcgatgcttgtcctgcgacgacgggctcgaacaatattgcgagcacggtttcaccgccacctataacggcccgatctacggctcgggcgagaacacctttggcggctattcggagaaaatcgtcgtcgacgcgcatttcgtgctggcgatccaccattctgagacgcagcttgccggagtcgcgccgctgctctgcgccggcatcaccacttggtcgccgctcaagcattggggtgtcggcccgggaaaatcggtcggcatcgtcggcatcggcgggctcggccatatgggggtcaagctcgcccatgcgctcggcgcccatgtcgtcgccttcaccacctcgccgtcaaagcgcgacgcggccctcgcgctcggcgccgacgaggtcgtcgtctccacagatcctgccgctatggcggcgcgggcgggaagcctcgacttcattctcgatacggtcgccgtcgcccatgacctcgacgcttatgtgaatctgttgaagcgcgatggcgctctggtgctcgtcggcgtgccggcgacgccgcatccctcgccatcggcgggcgggttgatcttcaagcggcgccaggtcgccggctcgctgatcggcggcgtaaaggagacgcaggagatgctcgacttctgcgccgagcgcggcattgtcgcggacatagagacgatcgccatgcagcagatcgagaccgcctatgcgcgcatgctgaagaatgatgtgaaataccgcttcgtcatcgacatggcgacgctgaaggcggcgtga

SEQ ID NO: 53 - protein, 342 aa, Methylococcus capsulatus
MKAWVIDRIGPLDSSRTLLRATDLPVPEPGPGEILLQVAVCGVCHTEIDEIEGRTAPPRLPVVPGHQAVGRIAALGSGVAEFALGDRVGVAWIFSACGECEFCRSGRENLCFAFCATGRDVDGGYAQYMTVPAAFAFRIPEGFTDAEAAPLLCAGAIGYRSLNLSGLKNGQPLGLTGFGASAHLVLMMARYRFPDSEVYVFARHPEERAFALQLGAVWAGDTADIAPAPLAAIIDTTPAWKPVVAALANLAPGGRLVVNAIRKAPDDRACLAELDYARHLWMEREIKSVANVARSDVAGFLALAAEMGIRPETEEYPFEDADRALLDLKQRRIRGAKVLRVT

SEQ ID NO: 54 - DNA, 1029 bp, Methylococcus capsulatus
  source 1..1029: mol_type = genomic DNA
atgaaagcttgggtgatcgaccgaatcggcccgctggactcgtcgcgaactctgctacgcgccaccgacctcccggtgccggagcccggccctggcgaaatcctgctgcaggtggcggtttgcggcgtctgccacaccgaaatcgacgagatcgagggccgcaccgcgccgccgcgcctgccggtcgtgcccggacaccaagcggtcggtcggatcgcggctctcggctccggcgtggcggaattcgctttgggcgaccgcgtcggcgtggcctggatcttttctgcctgcggagaatgcgaattctgccggtcgggacgggagaacctctgtttcgcattctgtgccaccgggcgcgatgtcgacggcggctacgcccagtacatgaccgtcccggcggcctttgctttccgcattccggagggattcaccgatgccgaagcggcgccgcttctgtgcgccggcgccatcggttaccgttcgctcaatctcagcgggctgaaaaacggccagccgctggggctcaccgggttcggggcttccgcccatctggtgctgatgatggcccggtaccggtttcccgattcggaagtctatgtctttgcgcgtcatcccgaggagcgcgcgttcgcgctgcagctgggcgcggtctgggccggcgacaccgcggacattgctcccgccccgctggccgccatcatcgacacgacgccggcgtggaagccggtggtcgcagcgctcgccaacctcgctcccggtggccggctggtcgttaatgcgatccgcaaggcgccggacgatcgcgcctgtctcgccgaactcgactatgcccggcacttgtggatggaacgggaaatcaagtcggtcgccaacgtggcgcgcagtgacgtggccgggttcctggcgctggcggcggaaatgggcatccgtcccgagacggaggagtacccgttcgaggatgccgaccgggcgctgctcgacctcaagcaacgccggattcgcggggcgaaggtgttgcgggtgacttga

SEQ ID NO: 55 - protein, 355 aa, Methylococcus capsulatus
MPTAKAYAAFSADSALAPFVLQRRDPLPQDIRIGILYCGVCHSDLHQARNEWNATTYPCVPGHEIVGKVLEVGRSVTKFKPGDTVAVGCMVDSCRTCPNCVDALEQHCEHGPVFTYNSPDPHGGGMTFGGYAESIVVDEAFVLRIPDGLDLAAAAPLLCAGITTYSPLRHWKVGAGQRVGVVGLGGLGHMALKFAHTFGAETVLFTTTPDKAEDARRLGADEVVVSRDPEAMARQAGRFDFILDTVSAPHDIDAYLNLLRRDGTLTLVGVPPQGVQVMPFSLIGGRRRLAGSLIGGIRETQEMLDFCGEHGIVCDIELIPIQGINDAFERMLKSDVKYRFVIDMATLNGESSGGR

SEQ ID NO: 56 - DNA, 1068 bp, Methylococcus capsulatus
  source 1..1068: mol_type = genomic DNA
atgcctacagccaaagcctatgccgctttttccgcagactcggcgctggcgccgttcgtcctgcagcggcgcgacccactgccccaggacatccgcatcggaatcctgtactgcggtgtctgccattccgacctgcaccaggcacgcaatgagtggaatgcgaccacatatccttgtgtgccaggccatgagatcgtcggcaaggtccttgaagtcggccgcagcgtgacgaagttcaagcccggcgacacggtcgcggtgggctgcatggtggattcctgccggacctgcccgaactgcgtggacgccctggaacagcactgcgagcacggccccgtcttcacctacaacagccccgatccgcacggcggcggcatgaccttcggtggctatgccgagagcatcgtggtcgacgaggccttcgtgctgcggataccggacggactggacctcgcggccgccgccccgctgttgtgcgccgggattaccacctattcgcccctgcggcactggaaagtgggggcgggtcagcgggtcggggtcgtcggtctgggtggactgggacacatggcgctcaagttcgcgcataccttcggcgccgaaacggtgctgttcacgacgacgccggacaaggcggaggatgcccgtcggctgggagcggacgaggtcgtcgtgtcgagggatcccgaggccatggcgcggcaggccggccggttcgatttcatcctcgacaccgtctcggcgccccatgacatcgatgcctatctgaacctgctgaggcgggacggcacgctgaccctggtcggcgtacctccgcaaggggtacaggtcatgcccttcagcctgatcggcgggcgccggcgactggctggttcattgatcggcggcatccgggaaacccaggagatgctggatttctgcggcgaacacggcatcgtctgcgacatcgagctgattccgatccaaggaatcaacgacgccttcgagcgcatgctcaaaagcgacgtgaaataccgtttcgtgatcgacatggcgacgctgaacggggagtcgtccggagggcgatga

SEQ ID NO: 57 - protein, 348 aa, Saccharomyces cerevisiae; identical to SEQ ID NO: 27

SEQ ID NO: 58 - DNA, 1047 bp, Saccharomyces cerevisiae
  source 1..1047: mol_type = genomic DNA
atgagcatccccgagacccagaaggccatcatcttctacgagagcaacggcaagctggaacataaggacatcccggtgcccaagcccaagccgaacgaactgctgatcaacgtgaagtacagcggcgtctgccacaccgacctgcacgcgtggcatggcgactggccgctgcccaccaagctgcccctggtgggcggccatgaaggcgccggcgtcgtggtcggcatgggcgagaacgtcaagggctggaagatcggcgactacgcgggcatcaagtggctgaacggcagctgcatggcctgcgagtattgcgaactgggcaacgaatcgaactgcccgcacgcggacctgtccggctacacccatgacggcagcttccaggagtatgccaccgcggacgccgtgcaggccgcgcacatcccgcagggcaccgacctggcggaggtggcccccatcctgtgcgccggcatcaccgtctacaaggcgctgaagagcgccaacctgcgcgcgggccattgggccgcgatctcgggcgccgccggtggcctgggctccctggccgtgcagtacgcgaaggcgatgggctaccgcgtcctgggcatcgacggcggtccgggcaaggaagagctgttcacctccctgggcggcgaagtgttcatcgacttcaccaaggagaaggacatcgtcagcgccgtggtcaaggcgaccaacggcggcgcccacggcatcatcaacgtgtcggtctccgaagccgcgatcgaggcgtcgacccgctactgccgggccaacggcaccgtggtcctggtgggcctgcccgcgggcgccaagtgcagctcggacgtcttcaaccatgtggtcaagagcatctcgatcgtgggctcgtatgtcggcaaccgcgccgacacccgcgaggccctggacttcttcgcccgtggcctggtcaagtccccgatcaaggtggtcggcctgtccagcctgcccgagatctacgaaaagatggagaagggccagatcgccggccgctatgtggtcgacacctccaagtga

SEQ ID NO: 59 - protein, 509 aa, Methylococcus capsulatus
MLQKYIEKILRARVYDVAQETPLDPAPGLSRRLDNTVLIKREDLQPVFSFKLRGAYNKIASLTPEARAAGVIAASAGNHAQGVALAAQRLGIRAVIVMPCTTPHIKVDAVRNRGGEVVLHGDAYDEAYEHALELARDQCLTFVHPYDDPEVIAGQGTIGMEILRQHQDAIHAIFVPVGGGGLIAGIAAYVKFVRPDIRVIGVEPVDSDCLHRALKAKRRVILKQVGLFADGVAVKQVGKEPFHLAHQWVDEVVTVDTDEICAAIKDIFDDTRSIAEPAGALGIAGLKKYVAETGIKNACLVAIESGANINFDRLRHVAERAEIGEKRELLLAVTIPERPGSFLEFCRVLGRRNITEFNYRFFDEKAAQVFVGLPVASGAIDRESLVREFERQGFGVLDLTGNELAIEHIRYMVGGHAPKLLDEQVYSFEFPERPGALLRFLSIMGGRWNISLFHYRNHGAAFGRVLMGIQVPKPERKAFREFLEAIGYAFKEETQNPAYRLFAGGSERG

SEQ ID NO: 60 - DNA, 1530 bp, Methylococcus capsulatus
  source 1..1530: mol_type = genomic DNA
atgctgcaaaaatacatagagaagattctgcgcgcccgtgtctacgacgttgcccaggagaccccgctggacccggcgcccggcctgtcgcggcggctggacaacacggtgctgatcaagcgcgaggacctgcagccggtgttctcgttcaagctgcgcggcgcctacaacaagatcgcctcgctcacacccgaggcgcgcgcggccggcgtgatcgcggcctccgccggcaaccacgcccagggcgtggcactggcggcgcagcggctgggcatccgcgccgtgatcgtgatgccttgcaccaccccgcatatcaaggtcgatgcggtgcgcaaccgaggcggtgaggtcgtactgcatggcgacgcctatgacgaagcctacgaacatgcgctggaactggcccgcgaccagtgcctgaccttcgtccacccctacgacgatccggaagtcatcgccgggcaaggcaccatcggcatggaaatcctgcgccagcaccaggacgccatccacgccatcttcgtgcctgtgggcggcggcggattgatcgccggcatcgccgcctacgtcaagttcgtgcgcccggacatccgcgtcatcggcgtggaaccagtggactccgactgcctgcaccgggcgctgaaagccaagcggcgggtgatcctgaagcaggtgggcctgttcgccgacggcgtcgcggtgaagcaggtcggcaaggaaccgttccatctcgcccaccagtgggtggacgaggtcgtgaccgtcgacaccgacgaaatctgcgccgccatcaaggacatcttcgacgacacccgctccatcgccgagccggcgggcgcgctgggcatcgccgggctcaagaaatacgtggccgaaacaggaatcaagaacgcgtgcctggtggcgatcgaaagcggcgccaacatcaacttcgaccggctgcgccacgtcgctgagcgcgccgagatcggcgaaaagcgcgaactgctgctggcagtgacgatccccgagcggcccggcagcttcctcgaattctgccgggtgctgggccgccgcaacatcaccgaattcaactaccgcttcttcgacgaaaaggccgcccaggtgttcgtcggcctcccggtggcgagcggcgcgatcgaccgcgaaagcctggtccgcgaattcgaacgccagggtttcggcgtgctcgacctgaccggcaacgaactcgccatcgaacacatccgctacatggtcggcggccacgcgccgaaactgctggacgaacaggtctacagcttcgaattccccgagcgacccggcgcgctgctgcgcttcctgtccatcatgggcgggcgctggaacatcagcctgttccattaccgcaaccacggcgccgccttcggccgggtactgatgggcatccaggtgccgaaaccggaacgcaaggccttccgggaattcctcgaagccatcggctacgccttcaaggaggaaacccaaaatcccgcctaccggctgttcgcggggggcagcgagcgggggtga

SEQ ID NO: 61 - protein, 514 aa, Methylococcus capsulatus
MHDRLIIFDTTLRDGEQSPGASMTRDEKVRIARALERLKVDVIEAGFPAASPGDFEAVQAVARTIKDSRVCGLARALDRDIDRAGEALKDAQRARIHTFIATSPIHMRHKLQMSPDQVVEYAVKAVKRARQYTDDVEFSPEDAGRSEEDFLCRILEAVIDAGATTLNIPDTVGYAFPEQFGHMIGRLIERIPNSDKAVFSVHCHNDLGLAVANSLAAVLHGARQVECTINGLGERAGNAALEEIVMAVRTRKDIFPCHTDIETREIVACSKLVSSITGFPIQPNKAIVGANAFAHESGIHQDGVLKSRETYEIMSAEDVGWSTNRMVLGKHSGRNAFRTRMQELGIEFASEEELNSVFQRFKVLADKKHEIFDEDLQALITEAGAEAEDERVKLVALRVCSETGEIPHAQVTIKVDNEERTGTSSGGGAVDASLKAIESLLHTDTALTLYSVNNITSGTDAQGEVTVRLEKGGRIVNGQGADTDIVIASAKAYVNAVNKLLAPIQRTHPQVGDV

SEQ ID NO: 62 - DNA, 1545 bp, Methylococcus capsulatus
  source 1..1545: mol_type = genomic DNA
atgcacgacagactgatcattttcgacacgaccttgcgcgacggagagcagagccccggcgcgtccatgacccgcgatgaaaaggtccgcatcgcccgggcgctggagcgtctgaaggtcgacgtcatcgaggcgggctttcccgccgccagccccggcgatttcgaggccgtccaggccgtggcccggaccatcaaggacagcagggtctgcggcctggcccgcgccctcgaccgcgacatcgaccgcgccggcgaagccctcaaggacgcccagcgcgcccgcatccacaccttcatcgccacctcgcccatccacatgcggcacaagctgcagatgtcgcccgaccaggtggtggaatacgcggtcaaggccgtcaagcgggcccgccagtacaccgacgacgtggaattctcgcccgaggacgccggacgctccgaggaggatttcctctgccgcatcctggaagccgtgatcgatgcgggggcgaccacgctgaacatccccgacaccgtcggctacgccttcccggaacagttcgggcacatgatcggccggctgatcgagcggattccgaactccgacaaggccgtgttctcggttcactgccacaacgacctgggactggcggtcgccaattcgctggccgccgtgctgcacggcgcgcgccaggtggaatgcaccatcaacgggctgggcgagcgggccggcaacgccgcgctggaagagatcgtcatggcggtgcgcacccgtaaagacatcttcccctgccacaccgacatcgagacacgggaaatcgtggcctgctccaaactggtctccagcatcaccggtttcccgatccagcccaacaaggccatcgtcggcgccaacgccttcgcccacgagtcgggcatccaccaggacggtgtgctcaagagccgggaaacctacgagatcatgagcgccgaggacgtggggtggagcaccaaccgcatggtgctgggcaaacattccggccgcaacgcgttccgtacccggatgcaggaactcggcatcgagttcgcctcggaagaggaactgaactcggtgttccagcgcttcaaggtgctggccgacaagaagcacgagatcttcgacgaggacctccaggccctcatcaccgaagccggcgcagaagccgaagacgaacgggtcaagctggtcgcgctgcgggtctgctcggaaacgggcgagattccccacgcccaggtcaccatcaaggtggacaacgaggaacgcaccggcacatcgagcggcggcggcgccgtggacgccagcctcaaggccatcgaatcgctgctgcacacggacaccgcgctgacgctgtactcggtcaacaacatcaccagcggcaccgacgcccagggcgaggtcaccgtgcggctcgagaaaggcgggcgcatcgtcaacggccagggcgccgataccgacatcgtgatcgcctcggccaaggcctacgtcaacgccgtgaacaagctgctggcgcccatccagcgcacccacccgcaagtcggggatgtgtga

SEQ ID NO: 63 - protein, 470 aa, Methylococcus capsulatus
MSGKTLYDKLWDDHVVHVDADGSCLIYIDRHLIHEVTSPQAFEGLRMAGRVPWRVDANLAVADHNVPTADRDRGIADPVSRLQVETLDKNCADFGITEFAMDDVRQGIVHVIGPEQGATLPGMTIVCGDSHTSTHGAFGALAFGIGTSEVEHVLATQCLVQRKAKNMLVRVQGKLAPGVTAKDLVLAVIGRIGTAGGTGYTIEFAGEAIRGLSMEGRMTVCNMAIEAGARAGLVAVDEVTLDYLEGRPFAPAGALWERAVEAWKDLHSDPDAVFDKVVEIDAASIKPQVTWGTSPEQVVPVDAEVPDPATEADPVRRESMERALQYMDLLPGTPIGAIRVDRVFIGSCTNARIEDLRAAAEVVRGHKRAASVKQALVVPGSGLVKRQAEQEGLDKVFLEAGFEWRDPGCSMCLAMNADRLEPGERCASTSNRNFEGRQGYGGRTHLVSPAMAAAAAIHGHFVDITEGGRA

SEQ ID NO: 64 - DNA, 1413 bp, Methylococcus capsulatus
  source 1..1413: mol_type = genomic DNA
atgagcggaaaaaccctttacgacaagctgtgggacgaccacgtcgtgcatgtcgatgcggacggatcgtgcctgatctacatcgatcgtcatctaatccacgaggtgacctcgcctcaggcattcgaagggctgcggatggcggggcgtgtaccctggcgggtggatgccaatcttgcggtggccgaccacaacgtccccaccgccgaccgcgacaggggtatcgccgatccggtgtcgcgcctgcaggtggaaaccctggacaagaactgcgccgatttcggcatcaccgaattcgcgatggacgacgtgcgccagggtatcgtgcatgtgatcgggcccgagcagggcgcgaccctgccgggcatgaccatcgtttgcggcgattcgcatacttcgactcacggtgctttcggggcgctcgccttcgggatcggcacttccgaggtcgagcacgtactggccacgcaatgcctggtgcagcgcaaggcgaagaacatgctggtccgcgtccagggcaagctggcgccgggcgtgacggcgaaagatctggtactggcggtcatcggccgtatcggaaccgccggcggcaccggctacaccatcgaattcgctggcgaagccattcgcggcctgtcgatggaaggccggatgacggtctgcaacatggcgatcgaggcgggcgcacgtgccggcctggtggcggtggacgaagtcacgctcgactatctcgagggccgcccgttcgctccggcgggcgcgttgtgggagcgggcggtcgaggcatggaaagacctgcacagcgatccggatgcggtattcgacaaggtcgtcgagatcgatgccgccagcatcaagccgcaggtgacctggggaacttcgccggaacaggtcgtgccggtggatgccgaggtgcccgacccggccacggaagccgatcccgtgcggcgggaaagcatggagcgggcgctgcagtacatggatctcctgccgggcacgccaatcggcgcgatccgggtcgatcgggtgttcatcggctcctgcaccaatgccaggatcgaggatctgcgcgccgcggcggaagtcgtccgggggcacaagcgcgctgccagcgtgaagcaggcactggtggtgcccggctcgggtttggtcaagcggcaggcggagcaggaggggctggacaaggtgttcctcgaggccggtttcgaatggcgcgacccgggttgttccatgtgtctggcgatgaacgccgaccgcctggaacccggcgagcgttgcgcctcgacctccaaccggaattttgaggggcgccagggctatggcgggcgtacccatctggtgagtccggccatggcggctgcggcggccattcacgggcatttcgtcgacatcaccgaaggagggcgcgcatga

SEQ ID NO: 65 - protein, 212 aa, Methylococcus capsulatus
MKPFKKFTSRVVPLDRANVDTDAIIPKQFLKSIRRSGFGPYLFDEWRYLDRGEPDMDCSHRPLNPEFVLNLPCYAGARILLARKNFGCGSSREHAPWALEDYGFRAIIAPSFADIFYNNCFKNGILPIVLDEATVDRLFSEAGPGFELTVDLESQTVATPFGETFHFDVDASRKHRLLNGLDDIGLTLQHADAIRAYEAARRKSAPWLFAVP

SEQ ID NO: 66 - DNA, 639 bp, Methylococcus capsulatus
  source 1..639: mol_type = genomic DNA
atgaagcctttcaagaaattcacttcgcgagtcgtgccgttggaccgcgccaatgtcgacaccgacgccatcattcccaagcagttcctgaagtccatccgccgcagcgggttcggtccctatctgttcgacgagtggcgttacctggaccgtggcgagcccgacatggattgcagccaccgtccgctcaacccggagttcgtgctcaacctgccctgttacgccggcgccaggatattgctggcccgcaagaacttcggctgtggctcctcgcgcgagcatgcgccctgggcgctggaggattacggcttccgcgccatcatcgcgccgagtttcgccgatatcttctacaacaactgcttcaagaacggcatcctgcccatcgtgctcgacgaggccacggtcgaccggctgtttagcgaggccgggcccggcttcgagctcaccgtcgacctggagtcgcagaccgtggcgacgccgttcggcgagaccttccatttcgacgtggatgcctcccgcaagcatcgtctgctgaacggcctggacgacatcggtctgacccttcagcatgccgatgccatccgcgcctacgaagccgcccgcaggaagtccgcaccctggctgtttgccgtcccttga

SEQ ID NO: 67 - protein, 559 aa, Zymomonas mobilis
MSYTVGTYLAALVQIGLKHHFAVAGDYNLVLLDNLLLNKNMEQVYCCNELNCGFSAEGYARAKADAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFQKKTALHRYLMGEVSYPGVEKTMKEADAVIALAPVFNDYSTTGWTDIPDPKKLVLAEPRSVVVNGVRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAARQQP

SEQ ID NO: 68 - DNA, 1905 bp, Zymomonas mobilis
  source 1..1905: mol_type = genomic DNA
tatcgctcatgatcgcgacatgttctgatattttcctctaaaaaagataaaaagtcttttcgcttcggcagaagaggttcatcatgaacaaaaattcggcatttttaaaaatgcctatagctaaatccggaacgacactttagaggtttctgggtcatcctgattcagacatagtgttttgaatatatggagtaagcaatgagttatactgtcggtacctatttagcggcgcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaagcggacgcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttccagaagaaaaccgcattacatcggtacctcatgggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggcgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgcccgccaacagccgtaagcctgttaacaagctcctctagttttt

SEQ ID NO: 69 - protein, 383 aa, Zymomonas mobilis
MASSTFYIPFVNEMGEGSLEKAIKDLNGSGFKNALIVSDAFMNKSGVVKQVADLLKAQGINSAVYDGVMPNPTVTAVLEGLKILKDNNSDFVISLGGGSPHDCAKAIALVATNGGEVKDYEGIDKSKKPALPLMSINTTAGTASEMTRFCIITDEVRHVKMAIVDRHVTPMVSVNDPLLMVGMPKGLTAATGMDALTHAFEAYSSTAATPITDACALKAASMIAKNLKTACDNGKDMPAREAMAYAQFLAGMAFNNASLGYVHAMAHQLGGYYNLPHGVCNAVLLPHVLAYNASVVAGRLKDVGVAMGLDIANLGDKEGAEATIQAVRDLAASIGIPANLTELGAKKEDVPLLADHALKDACALTNPRQGDQKEVEELFLSAF

SEQ ID NO: 70 - DNA, 1747 bp, Zymomonas mobilis
  source 1..1747: mol_type = genomic DNA
aaaggcaaaatcggtaaccacatctcaattattaaacaatacttcataataaaaagacaactttttcataatttgcataagtcttgatgtaaaaaatacatatttagaaagaacaagcagccttgctcatcaccgctgtcgcgagtagaaaaatctcggctttcagaaaaagaggccgcttcgttaaacagactataaatgtgctggaataaagcgaaccccttgatctgataaaactgatagacatattgcttttgcgctgcccgattgctgaaaatgcgtaaaaggtgattttactcgttttcaggaaaaactttgagaaaacgtctcgaaaacgggattaaaacgcaaaaacaatagaaagcgatttcgcgaaaatggttgttttcgggttgttgctttaaactagtatgtagggtgaggttatagctatggcttcttcaactttttatattcctttcgtcaacgaaatgggcgaaggttcgcttgaaaaagcaatcaaggatcttaacggcagcggctttaaaaatgcgctgatcgtttctgatgctttcatgaacaaatccggtgttgtgaagcaggttgctgacctgttgaaagcacagggtattaattctgctgtttatgatggcgttatgccgaacccgactgttaccgcagttctggaaggccttaagatcctgaaggataacaattcagacttcgtcatctccctcggtggtggttctccccatgactgcgccaaagccatcgctctggtcgcaaccaatggtggtgaagtcaaagactacgaaggtatcgacaaatctaagaaacctgccctgcctttgatgtcaatcaacacgacggctggtacggcttctgaaatgacgcgtttctgcatcatcactgatgaagtccgtcacgttaagatggccattgttgaccgtcacgttaccccgatggtttccgtcaacgatcctctgttgatggttggtatgccaaaaggcctgaccgccgccaccggtatggatgctctgacccacgcatttgaagcttattcttcaacggcagctactccgatcaccgatgcttgcgccttgaaggctgcgtccatgatcgctaagaatctgaagaccgcttgcgacaacggtaaggatatgccagctcgtgaagctatggcttatgcccaattcctcgctggtatggccttcaacaacgcttcgcttggttatgtccatgctatggctcaccagttgggcggctactacaacctgccgcatggtgtctgcaacgctgttctgcttccgcatgttctggcttataacgcctctgtcgttgctggtcgtctgaaagacgttggtgttgctatgggtctcgatatcgccaatctcggtgataaagaaggcgcagaagccaccattcaggctgttcgcgatctggctgcttccattggtattccagcaaatctgaccgagctgggtgctaagaaagaagatgtgccgcttcttgctgaccacgctctgaaagatgcttgtgctctgaccaacccgcgtcagggtgatcagaaagaagttgaagaactcttcctgagcgctttctaatttcaaaacaggaaaacggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccattt

SEQ ID NO: 71 - protein, 511 aa, Marinobacter algicola
MATQQQQNGASASGVLEQLRGKHVLITGTTGFLGKVVLEKLIRTVPDIGGIHLLIRGNKRHPAARERFLNEIASSSVFERLRHDDNEAFETFLEERVHCITGEVTESRFGLTPERFRALAGQVDAFINSAASVNFREELDKALKINTLCLENVAALAELNSAMAVIQVSTCYVNGKNSGQITESVIKPAGESIPRSTDGYYEIEELVHLLQDKISDVKARYSGKVLEKKLVDLGIREANNYGWSDTYTFTKWLGEQLLMKALSGRSLTIVRPSIIESALEEPSPGWIEGVKVADAIILAYAREKVSLFPGKRSGIIDVIPVDLVANSIILSLAEALSGSGQRRIYQCCSGGSNPISLGKFIDYLMAEAKTNYAAYDQLFYRRPTKPFVAVNRKLFDVVVGGMRVPLSIAGKAMRLAGQNRELKVLKNLDTTRSLATIFGFYTAPDYIFRNDSLMALASRMGELDRVLFPVDARQIDWQLYLCKIHLGGLNRYALKERKLYSLRAADTRKKA

SEQ ID NO: 72 - DNA, 1477 bp, Marinobacter algicola
  source 1..1477: mol_type = genomic DNA
atggcgacccagcagcagcagaacggcgcctcggcgagcggcgtcctggaacagttgcgcgggaagcatgtcctgataaccggtaccaccggtttccttggcaaggtagtcctggaaaagctgatccgcacagtcccggacatcggcggcatccacctcctgatccggggcaacaagaggcatccggccgcccgtgaacggttcttgaacgagatcgccagcagttcggtcttcgagcgtctgcgccacgacgacaacgaggccttcgaaaccttcctggaagaaagggtgcactgtataaccggagaggtcaccgagagtcgtttcggccttaccccggagcgcttccgcgcgctggcgggtcaggtggacgccttcatcaattcggccgcctccgtcaacttccgcgaggaactggacaaggcgctgaagatcaatacgctgtgcctggagaatgtcgcggcccttgctgaactcaacagtgcgatggcggtcatccaggtttcgacctgctacgttaacggcaagaatagcgggcagatcaccgaatcggtcatcaagcccgcgggggagtccatcccgcgtagcaccgatgggtactatgaaatcgaagaattggtgcacctgctgcaggacaaaatcagcgatgtgaaggcccgatactccgggaaggttctggaaaaaaaattggtggacctaggcatccgggaagccaataactacgggtggagcgatacatataccttcaccaagtggctgggcgaacagctcctcatgaaggccctgagcggcagatcgctgaccatcgtgcggccgtcgatcatcgagtcggcattggaagagcccagcccggggtggattgaaggcgtcaaggtcgccgatgccatcatactggcctacgcgagggagaaggtatcgctctttcctggcaagcggagcggcatcatcgacgtcatcccagtggatctggtggccaattcgatcattctgtccctggcggaggcgctctccggttcgggccagcggcgtatctatcagtgctgcagcggcggctcgaaccccatctccctcgggaagttcatcgactatctgatggcggaggcgaagaccaactacgcggcctacgatcagctgttctaccgccgccccaccaagccgttcgtggccgtcaaccgcaaactcttcgacgtcgtcgtgggcggcatgcgggtcccgctctcgatcgcgggcaaagccatgcgcctggcgggacaaaaccgcgaactgaaggtcctgaagaatctggatacgacccggtccctggccaccattttcgggttctacaccgctccggactacatctttcgcaatgacagcctgatggccctggcctcgcgcatgggcgagctggaccgcgtgttgttccccgttgacgcccgtcagatcgactggcagctgtatctgtgcaaaatccacctcggcgggctgaatcggtacg

SEQ ID NO: 73 - protein, 475 aa, Psychrobacter articus
MRLLTAVDQLFLLLESRKHPMHVGGLFLFELPENADISFVHQLVKQMQDSDVPPTFPFNQVLEHMMFWKEDKNFDVEHHLHHVALPKPARVRELLMYVSREHGRLLDRAMPLWECHVIEGIQPETEGSPERFALYFKIHHSLVDGIAAMRLVKKSLSQSPNEPVTLPIWSLMAHHRNQIDAIFPKERSALRILKEQVSTIKPVFTELLNNFKNYNDDSYVSTFDAPRSILNRRISASRRIAAQSYDIKRFNDIAERINISKNDVVLAVCSGAIRRYLISMDALPSKPLIAFVPMSLRTDDSIAGNQLSFVLANLGTHLDDPLSRIKLIHRSMNNSKRRFRRMNQAQVINYSIVSYAWEGINLATDLFPKKQAFNLIISNVPGSEKPLYWNGARLESLYPASIVFNGQAMNITLASYLDKMEFGITACSKALPHVQDMLMLIEEELQLLESVSKELEFNGITVKDKSEKKLKKLAP

SEQ ID NO: 74 - DNA, 1428 bp, Psychrobacter articus
  source 1..1428: mol_type = genomic DNA
atgcgcctgctgaccgccgtcgaccagctgttcctgctgctggagtcccgcaagcacccgatgcacgtgggcggcctgttcctgttcgaactgccggagaacgccgacatctcgttcgtccaccagctggtgaagcagatgcaggactccgacgtcccgcccaccttccccttcaaccaggtgctggaacacatgatgttctggaaggaggacaagaacttcgacgtcgaacaccatctgcaccatgtggccctgccgaagcccgcgcgcgtccgggagctgctgatgtacgtgtcccgcgaacacggccggctgctggaccgcgcgatgccgctgtgggaatgccatgtcatcgagggcatccagccggaaaccgagggcagccccgagcggttcgccctgtatttcaagatccaccattcgctggtcgacggcatcgccgcgatgcgcctggtgaagaagagcctgtcgcagtcgccgaacgaacccgtgaccctgccgatctggagcctgatggcccaccatcggaaccagatcgacgcgatcttccccaaggagcggagcgccctgcgcatcctgaaggaacaggtctcgaccatcaagccggtgttcaccgagctgctgaacaacttcaagaactacaacgacgactcgtatgtctccaccttcgacgcgccccgcagcatcctgaaccgccggatcagcgcctcgcgccggatcgccgcgcagtcgtacgacatcaagcggttcaacgacatcgccgaacgcatcaacatctccaagaacgacgtcgtgctggccgtgtgcagcggcgcgatccgccgctacctgatcagcatggacgcgctgccgagcaagcccctgatcgccttcgtcccgatgtcgctgcgcaccgacgactccatcgcgggcaaccagctgtcgttcgtgctggccaacctgggcacccacctggacgaccccctgtcccggatcaagctgatccatcgctccatgaacaacagcaagcgccggttccgccggatgaaccaggcccaggtcatcaactacagcatcgtgtcgtatgcctgggagggcatcaacctggcgaccgacctgttcccgaagaagcaggccttcaacctgatcatctcgaacgtgccgggcagcgagaagcccctgtactggaacggcgcgcgcctggaaagcctgtatccggcctcgatcgtgttcaacggccaggccatgaacatcaccctggcgtcctacctggacaagatggagttcggcatcaccgcctgcagcaaggcgctgccgcacgtccaggacatgctgatgctgatcgaggaagagctgcagctgctggagtccgtcagcaaggaactggagttcaacggcatcaccgtgaaggacaagtcggaaaagaagctgaagaagctggccccgtga

SEQ ID NO: 75 - protein, 461 aa, Rhodococcus opacus
MSVMSPTEAMFVLFETPSHPMHMGALELFEPPRESGPDHARLMFEALISQEGASDTFRRRAVRPLRGASYPWWSVDDRVDLGYHVRHTAVPGRGRMEDLLSLVSQMHGMPLDPQHPMWEIHVIEGLADGRTAVFSKIHLSLMDGPAGLRLLHHALSTDPDARDCPAPWTPGVSGTSRRESALPVAAVRAGVRAATSIVGVLPALAKVAYDGVRDQHLTLPLQSPPTMLNVPVGRARKLAARSWPIRRLVSVAAAARTTINAVVLAMCSGALRHYLVEQYALPEAPLTAMLPVPLDLGGTMIGPRGRDHGVGAMVVGLATDEADPAARLARISESVEHTNRVFGALSHTQFQVMSALAISPILLEPVRRFVDDTPPPFNVMISYMPGPSRPRYWNGARLDAVYPAPTVLGGQALSITLTSRSGQLDVGVVGDRQAVPHLQRIITHLETSLTDLENAVAASGT

SEQ ID NO: 76 - DNA, 1386 bp, Rhodococcus opacus
  source 1..1386: mol_type = genomic DNA
atgtccgtgatgtccccgaccgaggcgatgttcgtcctgttcgagaccccgagccacccgatgcacatgggcgcgctggagctgttcgagccgccgcgcgagtcgggcccggaccacgcccgcctgatgttcgaggcgctgatctcccaggaaggcgccagcgacaccttccgccggcgcgccgtccggccgctgcgcggcgcgtcgtacccctggtggtccgtcgacgaccgggtggacctgggctatcacgtccgccataccgccgtgccgggccggggccgcatggaggacctgctgtcgctggtgtcccagatgcacggcatgcccctggacccgcagcaccccatgtgggagatccatgtcatcgaaggcctggccgacggccgcaccgcggtgttcagcaagatccatctgtcgctgatggacggcccggccggcctgcggctgctgcaccatgcgctgagcaccgacccggacgcccgcgactgccccgcgccgtggacccccggcgtcagcggcacctcgcggcgcgaatcggccctgccggtcgccgcggtgcgggcgggcgtgcgcgccgcgacctccatcgtcggcgtgctgcccgccctggcgaaggtcgcctacgacggcgtgcgggaccagcacctgaccctgccgctgcagagcccgcccaccatgctgaacgtccccgtgggccgggcccgcaagctggccgcgcggagctggccgatccggcgcctggtctcggtggccgcggccgcgcgcaccaccatcaacgccgtcgtgctggcgatgtgctcgggcgccctgcgccactacctggtcgagcagtatgccctgccggaagcgcccctgaccgccatgctgcccgtgccgctggacctgggcggcaccatgatcggcccgcgtggccgcgaccacggcgtcggcgcgatggtcgtgggcctggcgaccgacgaggccgaccccgccgcgcggctggcccgcatcagcgagtcggtcgaacacaccaaccgcgtgttcggcgcgctgtcccatacccagttccaggtcatgtccgccctggcgatcagcccgatcctgctggaacccgtccggcgcttcgtggacgacaccccgcccccgttcaacgtgatgatctcgtacatgccgggtccgtcccggccgcgctattggaacggcgcgcggctggacgccgtctaccccgcgccgaccgtgctgggcggccaggccctgagcatcaccctgacctcccgcagcggccagctggacgtcggcgtcgtgggcgaccggcaggccgtgccgcacctgcagcgcatcatcacccatctggagacctccctgaccgacctggaaaacgccgtggccgcgagcggcacctga

SEQ ID NO: 77 - protein, 451 aa, Rhodococcus opacus
MPVTDSIFLLGESREHPMHVGSLELFTPPDDAGPDYVKSMHETLLKHTDVDPTFRKKPAGPVGSLGNVWWADESDVDLEYHVRHSALPAPYRVRELLTLTSRLHGTLLDRHRPLWEMYLIEGLSDGRFAIYTKLHHSLMDGVSGLRLLMRTLSTDPDVRDAPPPWNLPRPAAANGAAPDLWSVVNGVRRTVGDVAGLAPASLRIARTAMGQHDMRFPYEAPRTMLNVPIGGARRFAAQSWPLERVHAVRKAAGVSVNDVVMAMCAGALRGYLEEQNALPDEPLIAMVPVSLRDEQQADAGGNAVGVTLCNLATDVDDPAERLTAISASMSQGKELFGSLTSMQALAWSAVNMSPIALTPVPGFVRFTPPPFNVIISNVPGPRKTMYWNGSRLDGIYPTSVVLDGQALNITLTTNGGNLDFGVIGCRRSVPSLQRILFYLEAALGELEAALL

SEQ ID NO: 78 - DNA, 1356 bp, Rhodococcus opacus
  source 1..1356: mol_type = genomic DNA
atgccggtcaccgactccatcttcctgctgggcgaaagccgcgagcacccgatgcacgtgggctccctggaactgttcacccccccggacgacgccggcccggactacgtcaagtcgatgcacgagaccctgctgaagcataccgacgtggaccccaccttccgcaagaagccggcgggccccgtcggctcgctgggcaacgtgtggtgggccgacgagtccgacgtcgacctggaataccacgtgcgccatagcgcgctgccggccccctatcgcgtccgggaactgctgaccctgacctcgcggctgcacggcaccctgctggaccgccatcggccgctgtgggagatgtacctgatcgaaggcctgagcgacggccgcttcgccatctataccaagctgcaccatagcctgatggacggcgtctcgggcctgcgcctgctgatgcggaccctgtcgaccgacccggacgtgcgcgacgccccgcccccgtggaacctgccgcggcccgccgcggccaacggcgcggccccggacctgtggtcggtcgtgaacggcgtccgccggaccgtcggcgacgtggccggcctggcgcccgcctccctgcgcatcgcgcggaccgcgatgggccagcacgacatgcgcttcccgtacgaggcgccccggaccatgctgaacgtgccgatcggcggcgcccgccggttcgcggcccagtcctggcccctggaacgcgtccatgccgtgcggaaggcggccggcgtcagcgtgaacgacgtcgtgatggccatgtgcgcgggcgccctgcgcggctatctggaggaacagaacgcgctgccggacgagcccctgatcgcgatggtcccggtgtccctgcgggacgaacagcaggcggacgccggcggcaacgccgtcggcgtgaccctgtgcaacctggcgaccgacgtcgacgaccccgccgagcgcctgaccgcgatcagcgcctcgatgtcccagggcaaggaactgttcggcagcctgacctcgatgcaggcgctggcctggtcggcggtgaacatgtccccgatcgccctgaccccggtccccggcttcgtgcggttcacccccccgcccttcaacgtcatcatcagcaacgtgccgggcccccgcaagaccatgtactggaacggctcccggctggacggcatctatccgaccagcgtcgtgctggacggccaggccctgaacatcaccctgaccaccaacggcggcaacctggacttcggcgtcatcggctgccgccggtccgtgccgagcctgcagcgcatcctgttctacctggaagcggccctgggcgagctggaagcggccctgctgtga

SEQ ID NO: 79 - protein, 464 aa, Rhodococcus opacus
MPLPMSPLDSMFLLGESREHPMHVGGVEIFQLPEGADTYDMRAMLDRALADGDGIVTPRLAKRARRSFSSLGQWSWETVDDIDLGHHIRHDALPAPGGEAELMALCSRLHGSLLDRSRPLWEMHLIEGLSDGRFAVYTKIHHAVADGVTAMKMLRNALSENSDDRDVPAPWQPRGPRPQRTPSSKGFSLSGLAGSTLRTARETVGEVAGLVPALAGTVSRAFRDQGGPLALSAPKTPFNVPITGARQFAAQSWPLERLRLVAKLSDSTINDVVLAMSSGALRSYLEDQNALPADPLIAMVPVSLKSQREAATGNNIGVLMCNLGTHLREPADRLETIRTSMREGKEAYGSMTATQILAMSALGAAPIGASMLFGHNSRVRPPFNLIISNVPGPSSPLYWNGARLDAIYPLSVPVDGQGLNITCTSNDDIISFGVTGCRSAVPDLKSIPARLGHELRALERAVGI

SEQ ID NO: 80 - DNA, 1395 bp, Rhodococcus opacus
  source 1..1395: mol_type = genomic DNA
atgcccctgccgatgtcccccctggactccatgttcctgctgggcgaaagccgcgagcacccgatgcacgtgggcggcgtcgaaatcttccagctgcccgagggcgccgacacctacgacatgcgggcgatgctggaccgcgccctggcggacggcgacggcatcgtcaccccgcggctggccaagcgcgcgcgccggtcgttcagctcgctgggccagtggtcctgggaaaccgtggacgacatcgacctgggccaccatatccggcacgacgccctgccggcccctggcggcgaggccgaactgatggcgctgtgctcgcgcctgcacggctccctgctggaccgcagccggccgctgtgggagatgcatctgatcgaaggcctgagcgacggccgcttcgccgtctataccaagatccaccatgccgtcgcggacggcgtgaccgccatgaagatgctgcggaacgcgctgagcgagaactcggacgaccgcgacgtgccggccccctggcagccgcgtggcccgcggccccagcgcaccccctccagcaagggcttctccctgagcggcctggccggctcgaccctgcggaccgcgcgcgagaccgtcggcgaagtggccggcctggtcccggccctggcgggcaccgtgagccgggccttccgcgaccagggcggcccgctggccctgtcggcgccgaagacccccttcaacgtccccatcaccggcgcccgccagttcgccgcgcagtcgtggccgctggaacgcctgcggctggtggccaagctgtcggactccaccatcaacgacgtcgtgctggccatgtcgtccggcgcgctgcggtcctacctggaggaccagaacgccctgccggcggaccccctgatcgcgatggtcccggtgtccctgaagagccagcgcgaagccgcgaccggcaacaacatcggcgtcctgatgtgcaacctgggcacccacctgcgggagccggccgaccgcctggaaaccatccggaccagcatgcgcgagggcaaggaagcctatggctcgatgaccgcgacccagatcctggccatgtccgcgctgggcgccgcgccgatcggcgccagcatgctgttcggccataactcgcgcgtccggccgcccttcaacctgatcatctccaacgtgccgggccccagctcgccgctgtactggaacggcgcccgcctggacgcgatctatccgctgagcgtccccgtggacggccagggcctgaacatcacctgcacctcgaacgacgacatcatctccttcggcgtcaccggctgccggtccgccgtgccggacctgaagagcatccccgcgcgcctgggccatgagctgcgggccctggaacgcgcggtgggcatctga

SEQ ID NO: 81 - protein, 468 aa, Rhodococcus opacus
MAPTDSLFLLGESREHPMHVGGLAVFTPAEGSSAADVRAMFDAALVGDRVAAPFRKRARRSVTSLGQWGWDTLRDDEVDLEHHVRRDALPQPGGMAELMTLVSRLHGTLLDRSRPLWEMHLIEGLADGRYAVYTKIHHALADGASAMRLLRDSMSEDPHRRNMPTPWQPRNPLSAVPDAGVAVTPGPGSALPAMAWDAARSAAGEVAGLLPAALGTVDRALHGKGGALSLTAPHTLFNVPISGARHVAARSFPIERIRLLAKHADATINDIVLTMCAGTLRAYLHTRDALPDNPLIAMVPVSLRAPETGTGDRAPGGNRVGVLMCNLATHLPDPAHRLETVRNCMNEGKAALQAMSPAQVLAMSALGAAPLGVEMFLGRRGPLRPPFNVVISNVAGPRTPLYWNGARLESLYPLSIPTTGQALNITCTSSDDQIVFGLTGCRRTVPDLHPMLDQLDAELDLLETAVGL

SEQ ID NO: 82 - DNA, 1407 bp, Rhodococcus opacus
  source 1..1407: mol_type = genomic DNA
atggccccgaccgactccctgttcctgctgggcgaatcccgcgagcacccgatgcacgtgggcggcctggcggtcttcaccccggcggagggcagctcggccgcggacgtccgcgccatgttcgacgccgcgctggtcggcgaccgggtggccgcgccgttccgcaagcgggcccgccggagcgtgacctcgctgggccagtggggctgggacaccctgcgcgacgacgaggtcgacctggaacaccatgtgcgccgggacgccctgccgcagccgggtggcatggcggaactgatgaccctggtctcccgcctgcatggcaccctgctggaccgcagccggccgctgtgggagatgcacctgatcgaaggcctggccgacggccggtacgcggtgtataccaagatccaccatgccctggcggacggcgccagcgcgatgcgcctgctgcgggactcgatgtccgaggacccgcatcgccggaacatgccgaccccctggcagccgcgcaaccccctgtcggccgtcccggacgccggcgtcgcggtgacccccggccccggcagcgccctgcccgcgatggcctgggacgccgcgcgctccgccgcgggcgaagtcgccggcctgctgccggccgcgctgggcaccgtggaccgggccctgcacggcaagggcggcgccctgtccctgaccgcgccgcataccctgttcaacgtccccatcagcggcgcccgccacgtggccgcgcggtcgttcccgatcgagcgcatccggctgctggccaagcatgccgacgcgaccatcaacgacatcgtgctgaccatgtgcgccggcaccctgcgcgcgtacctgcacacccgcgacgccctgccggacaaccccctgatcgcgatggtcccggtgagcctgcgcgcccccgaaaccggcaccggcgaccgcgcccctggcggcaaccgggtcggcgtgctgatgtgcaacctggccacccacctgccggaccccgcgcatcgcctggagaccgtccggaactgcatgaacgaaggcaaggccgcgctgcaggccatgtcgccggcgcaggtcctggccatgtccgcgctgggcgccgcgccgctgggcgtggagatgttcctgggccgccggggccccctgcgcccgcccttcaacgtcgtgatctcgaacgtggcgggcccgcgcacccccctgtactggaacggcgcccggctggaatccctgtatccgctgagcatccccaccaccggccaggccctgaacatcacctgcacctccagcgacgaccagatcgtcttcggcctgaccggctgccgccggaccgtgccggacctgcaccccatgctggaccagctggacgcggagctggacctgctggaaaccgcggtcggcctgtga